(19) United States
(12) Patent Application Publication
(10) Pub. No.: US 2024/0199534 A1
(43) Pub. Date: Jun. 20, 2024

(54) CYCLOPROPANE ANALOGUES OF N-(TRANS-4-HYDROXYCYCLOHEXYL)-6-PHENYLHEXANAMIDE AND RELATED COMPOUNDS

(71) Applicant: Mitochondria Emotion, Inc., Cambridge, MA (US)

(72) Inventor: Gerald W. DORN, II, Sullivan's Island, SC (US)

(21) Appl. No.: 18/282,158

(22) PCT Filed: Mar. 21, 2022
(86) PCT No.: PCT/US2022/021210

§ 371 Date: Sep. 14, 2023

Related U.S. Application Data
(60) Provisional application No. 63/163,392, filed on Mar. 19, 2021.

(51) Int. Cl. C07C 233/23; A61P 25/28; C07C 233/60; C07C 235/40; C07C 323/60

(52) U.S. Cl. CPC C07C 233/23; A61P 25/28; C07C 233/60; C07C 235/40; C07C 323/60; C07C 2601/02; C07C 2601/14

(57) ABSTRACT

The present disclosure relates to compounds of Formula (I):

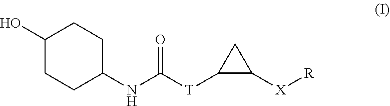

or pharmaceutically acceptable salts thereof. The present disclosure also relates to uses of the compounds, e.g., in treating or preventing diseases, disorders, or conditions (e.g., associated with mitochondria).

### RELATED APPLICATIONS

The present application claims benefit of U.S. Provisional Application No. 63/163,392, filed Mar. 19, 2021, which is hereby incorporated by reference in its entirety.

### BACKGROUND

Mitochondrial dysfunction may contribute to various types of neurodegenerative diseases. Defective mitochondrial fusion or fission may be especially problematic in this regard, especially when imbalanced fusion and fission lead to mitochondrial fragmentation. Among the many neurodegenerative diseases in which mitochondrial dysfunction has been implicated include, for example, Charcot-Marie-Tooth disease, amyotrophic lateral sclerosis (ALS), and Huntington's disease.

Mitochondrial fusion is initiated by outer mitochondrial membrane-embedded mitofusin (MFN) proteins whose extra-organelle domains extend across cytosolic space to interact with counterparts on neighboring mitochondria. The physically linked organelles create oligomers of varying sizes. Mitofusins subsequently induce outer mitochondrial membrane fusion mediated by catalytic GTPase. Aberrant mitofusin activity is believed to be a primary contributor to mitochondrial-based neurodegenerative diseases. For these reasons, mitofusins are attractive targets for drug discovery.

There remains a need for new compounds that target mitofusins. The present disclosure addresses the need.

### SUMMARY

In some aspects, the present disclosure features a compound to Formula (I):

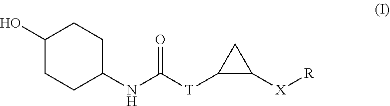

or a pharmaceutically acceptable salt thereof, wherein

T is absent, C₁-C₅ alkylene, or 1- to 5-membered heteroalkylene, wherein the C₁-C₅ alkylene or 1- to 5-membered heteroalkylene is optionally substituted with one or more R^{T}:

each R^{T} independent is halogen, cyano, —OR^{T1}, —N(R^{T1})₂, or C₃-C₁₀ cycloalkyl: or two R^{T}, together with the atom they attach to, form C₃-C₁₀ cycloalkyl or 3- to 10-membered heterocycloalkyl:

each R^{T1} independent is H or C₁-C₆ alkyl:

X is C₂-C₅ alkylene or 2- to 5-membered heteroalkylene, wherein the C₂-C₅ alkylene or 2- to 5-membered heteroalkylene is optionally substituted with one or more R^{X};

each R^{X} independent is halogen, cyano, —OR^{X1}, —N(R^{X1})₂, or C₃-C₁₀ cycloalkyl; or two R^{X}, together with the atom they attach to, form C₃-C₁₀ cycloalkyl or 3- to 10-membered heterocycloalkyl;

each R^{X1} independent is H or C₁-C₆ alkyl;

R is C₆-C₁₀ aryl or 5- to 10-membered heteroaryl, wherein the C₆-C₁₀ aryl or 5- to 10-membered heteroaryl is optionally substituted with one or more halogen, cyano, —OR^{S}, —N(R^{S})₂, or C₃-C₁₀ cycloalkyl; and

each R^{S} independent is H or C₁-C₆ alkyl.

In some aspects, the present disclosure provides an isotopic derivative of a compound described herein.

In some aspects, the present disclosure provides a method of preparing a compound described herein.

In some aspects, the present disclosure features a pharmaceutical composition comprising any compound described herein and a pharmaceutically acceptable excipient.

In some aspects, the present disclosure features a method of treating diseases, disorders, or conditions, comprising administering to a subject in need thereof any compound described herein in a pharmaceutical composition.

In some aspects, the present disclosure features any compound described herein in a pharmaceutical composition for use for treating diseases, disorders, or conditions, comprising administering to a subject in need thereof.

In some aspects, the present disclosure features use of any compound described herein in a pharmaceutical composition in the manufacture of a medicament for treating diseases, disorders, or conditions, comprising administering to a subject in need thereof.

In some aspects, the present disclosure features a method of activating mitofusin in a subject, comprising administering the compound or the pharmaceutical composition of any one of the preceding claims.

In some aspects, the present disclosure features any compound described herein in a pharmaceutical composition for use in activating mitofusin in a subject.

In some aspects, the present disclosure features use of the any compound described herein in a pharmaceutical composition in the manufacture of a medicament for activating mitofusin in a subject.

Unless otherwise defined, all technical and scientific terms used herein have the same meaning as commonly understood by one of ordinary skill in the art to which this disclosure belongs. In the specification, the singular forms also include the plural unless the context clearly dictates otherwise. Although methods and materials similar or equivalent to those described herein can be used in the practice or testing of the present disclosure, suitable methods and materials are described below. All publications, patent applications, patents and other references mentioned herein are incorporated by reference. The references cited herein are not admitted to be prior art to the claimed invention. In the case of conflict, the present specification, including definitions, will control. In addition, the materials, methods and examples are illustrative only and are not intended to be limiting. In the case of conflict between the chemical structures and names of the compounds disclosed herein, the chemical structures will control.

Other features and advantages of the disclosure will be apparent from the following detailed description and claims.

### BRIEF DESCRIPTION OF THE DRAWINGS

The following figures are included to illustrate certain aspects of the present disclosure, and should not be viewed as exclusive embodiments. The subject matter disclosed is capable of considerable modifications, alterations, combinations, and equivalents in form and function, as will occur to one having ordinary skill in the art and having the benefit of this disclosure.

FIG. 1 shows a representative HPLC chromatogram of the chiral separation of Compounds 2A and 2B.

FIGS. 2A and 2B show illustrative dose-response curves for Compounds 2A and 2B in comparison to Compound 6 for activity against MFN1 knockout MEFs and MFN2 knockout MEFs.

FIGS. 3A and 3B show corresponding illustrative plots of mitochondrial aspect ratio obtained in the presence of Compounds 2A and 2B in comparison to Compound 6 and DMSO vehicle.

FIG. 4 shows dose-response curves for Compounds 4A and 4B in comparison to Compound 1 for activity against MFN2 knockout MEFs.

FIG. 5 is an illustrative x-ray powder diffraction pattern for Compounds 4A and 4B.

FIGS. 6A and 6B show illustrative polarized light microscopy images of crystals of Compounds 4A and 4B.

FIGS. 7A and 7B show ORTEP diagrams representative of the single-crystal x-ray crystallographic structures of Compounds 4A and 4B, respectively.

FIG. 8 shows a packing diagram for Compound 4A.

FIG. 9 shows x-ray powder diffraction data for as-obtained, microcrystalline Compound 4A in comparison to simulated x-ray powder diffraction data obtained from the single-crystal x-ray crystallographic data of Compound 4A.

FIG. 10A shows numbers of mitochondria in sciatic nerve. FIG. 10B shows the mitochondria area of axonal mitochondria. FIG. 10C shows sciatic nerve ROS levels measured with 4-HNE.

FIG. 11A shows the sciatic nerve axon area. FIG. 11B shows damaged axons in sciatic nerve. FIG. 11C shows demyelinated axons in sciatic nerve. FIG. 11D shows apoptotic neurons in spinal cord.

FIG. 12A shows quantitative data regarding COX IV/AchR pixel intensity. Gastrocnemius neuromuscular junctions were labelled with anti-acetylcholine receptor (AchR) and mitochondrial cytochrome oxidase (COX).

FIG. 12B shows quantitative data regarding reduced area and central nuclear positioning. Wheat germ agglutinin (WGA) stained gastrocnemius sections showing myocyte atrophy and central nuclear positioning.

FIG. 12C shows the intensity of gastrocnemius sections stained for ROS with 4-HNE.

FIG. 12D shows succinate dehydrogenase (SDH)/cytochrome oxidase (COX) activities in gastrocnemius myocytes. Mean±SEM: *=p<0.05 vs wild-type (WT) normal control; #=p<0.05 vs vehicle-treated ALS (ALS) by ANOVA.

FIGS. 13A shows mouse SOD1 G93A DRG neurons stained for mitochondria and mitochondrial ROS. FIGS. 13B-C show quantitative data for TUNEL apoptosis stain and propidium iodide necrosis stain. FIGS. 13D and 13E shows quantitative data for mitochondria within DRG neuronal processes. FIG. 13F shows results of Seahorse oxygen consumption studies in ALS SOD1 I113T reprogrammed neurons. Inset shows ATP-linked respiration. Data are mean±SEM; *=p<0.05 vs wild-type (WT) normal control; #=p<0.05 vs DMSO-treated ALS by ANOVA.

FIG. 14 is a graph showing the MFN2 altering activity of exemplary compounds. The graph shows results of FRET studies comparing MFN2 conformation altering activities of prototype mitofusin activators 1 and 2 with Compound Nos. 2A and 2B (with all compounds added to a final concentration of 1 μM; assays were performed after 4 h). FRET assays were performed on isolated mitochondria, whereas assessments of mitochondrial elongation were performed in intact cells.

FIGS. 15A-15G are a set of graphs showing pharmacodynamic and therapeutic effects of 5 vs 2 in murine ALS. FIG. 15A shows representative kymographs for wild-type (WT) and ALS SOD1G93A mice (ALS) 12 h after oral administration of Compound 2 or vehicle. FIG. 15B shows time-dependent pharmacokinetics/pharmacodynamics of Compound 2 after single oral doses (60 mg/kg); the curved data line and left vertical axis show mitochondrial motility after 5 in ALS mouse sciatic nerve axons. FIG. 15C shows time-dependent pharmacokinetics/pharmacodynamics of Compound 1 after single oral doses (60 mg/kg); the curved data line and left vertical axis show mitochondrial motility in CMT2A mouse sciatic nerve axons. For FIGS. 15B and 15C, each point represents a single neuronal axon from two or three mice per time point. The straight data line and right vertical axes show corresponding plasma levels (n=5 per time point; means±SD). The dotted line designated “normal motility” is the mean value for WT; the dashed line designated “ALS motility” is the mean value for untreated ALS. FIG. 15D shows comparative pharmacodynamics of Compound 2 and Compound 1. FIG. 15E shows the effects of Compound 2 and Compound 1 on the neuromuscular dysfunction score (ledge test, hindlimb test, gait, kyphosis) in a proof-of-concept study of ALS mice. P values by ANOVA.

### DETAILED DESCRIPTION

Without wishing to be bound by theory, it is understood that the compounds disclosed herein may be effective in activating mitofusin. Thus, the compounds may be useful for treating various diseases and disorders, including mitochondria associated diseases, disorders, or conditions.

Various N-(cycloalkyl or heterocycloalkyl)-6-phenylhexanamide compounds may be potent mitofusin activators (U.S. Patent Application Publication 2020/0345669). N-(trans-4-hydroxycyclohexyl)-6-phenylhexanamide (Compound 1) could be a particularly potent example of a mitofusin activator (U.S. Patent Application Publication 2020/0345668).

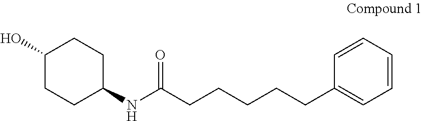 [SMILES: O=C(CCCCCc1ccccc1)N[C@H]1CC[C@H](O)CC1]

N-(trans-4-hydroxycyclohexyl)-6-phenylhexanamide

It was discovered that by introducing rigidity into the methylene chain extending between the amide carbonyl and the phenyl ring of Compound 1, the plasma half-life and neurological bioavailability may be significantly improved. A particularly efficacious mitofusin activator may be obtained by fusing the two methylene groups adjacent to the amide carbonyl together as a cyclopropyl group (cyclopropane ring), the structure of which is shown in Compound 2.

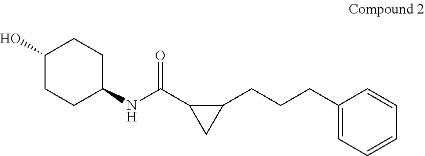 [SMILES: O=C(N[C@H]1CC[C@H](O)CC1)C1CC1CCCc1ccccc1]

N-((1r,4r)-4-hydroxycyclohexyl)-2-(3-phenylpropyl)cyclopropane-1-carboxamide

It was further discovered that particular stereoisomeric configurations upon the cyclopropane ring maintained activity toward mitofusin activation. In particular, the (R,R) configuration of Compound 2 is active toward promoting mitofusin activation, whereas the corresponding (S,S) configuration of Compound 2 is inactive. These compounds are represented by the structures shown in Compounds 2A and 2B below.

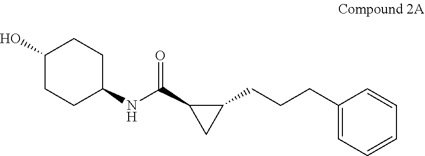 [SMILES: O=C(N[C@H]1CC[C@H](O)CC1)[C@@H]1C[C@H]1CCCc1ccccc1]

(1R,2R)-N-((1r,4R)-4-hydroxycyclohexyl)-2-(3-phenylpropyl)cyclopropane-1-carboxamide

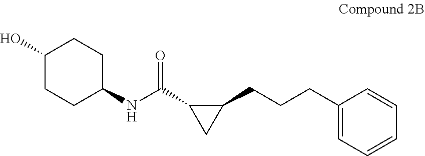 [SMILES: O=C(N[C@H]1CC[C@H](O)CC1)[C@H]1C[C@@H]1CCCc1ccccc1]

(1S,2S)-N-((1r,4R)-4-hydroxycyclohexyl)-2-(3-phenylpropyl)cyclopropane-1-carboxamide

Compounds of the disclosure also include Compounds 4A, 5A, 4B, and 5B.

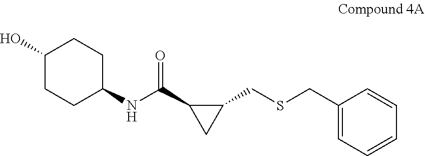 [SMILES: O=C(N[C@H]1CC[C@H](O)CC1)[C@@H]1C[C@H]1CSCc1ccccc1]

(1R,2R)-2-((benzylthio)methyl)-N-((1r,4R)-4-hydroxycyclohexyl)cyclopropane-1-carboxamide

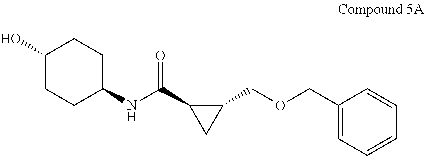 [SMILES: O=C(N[C@H]1CC[C@H](O)CC1)[C@@H]1C[C@H]1COCc1ccccc1]

(1R,2R)-2-((benzyloxy)methyl)-N-((1r,4R)-4-hydroxycyclohexyl)cyclopropane-1-carboxamide

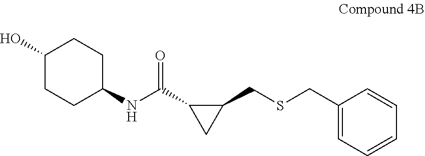 [SMILES: O=C(N[C@H]1CC[C@H](O)CC1)[C@H]1C[C@@H]1CSCc1ccccc1]

(1S,2S)-2-((benzylthio)methyl)-N-((1r,4R)-4-hydroxycyclohexyl)cyclopropane-1-carboxamide

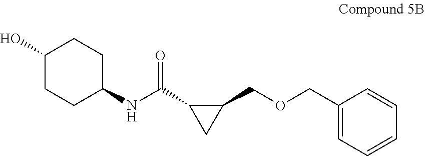 [SMILES: O=C(N[C@H]1CC[C@H](O)CC1)[C@H]1C[C@@H]1COCc1ccccc1]

(1S,2S)-2-((benzyloxy)methyl)-N-((1r,4R)-4-hydroxycyclohexyl)cyclopropane-1-carboxamide.

### Compounds of the Present Disclosure

Any structural feature described herein (e.g., for any exemplary formula described herein) can be used in combination with any other structural feature(s) described for any exemplary formula described herein.

In some aspects, the present disclosure features a compound of Formula (I):

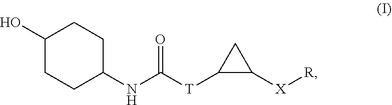

or a pharmaceutically acceptable salt thereof, wherein

T is absent, C₁-C₅ alkylene, or 2- to 5-membered heteroalkylene, wherein the C₁-C₅ alkylene or 1- to 5-membered heteroalkylene is optionally substituted with one or more R^{T};

each R^{T} independent is halogen, cyano, —OR^{T1}, —N(R^{T1})₂, oxo, C₁-C₁₀ alkyl, or C₃-C₁₀ cycloalkyl; or two R^{T}, together with the atom they attach to, form C₃-C₁₀ cycloalkyl or 3- to 10-membered heterocycloalkyl;

each R^{T1} independent is H or C₁-C₆ alkyl;

X is C₂-C₅ alkylene or 2- to 5-membered heteroalkylene, wherein the C₂-C₅ alkylene or 2- to 5-membered heteroalkylene is optionally substituted with one or more R^{X};

each R^{X} independent is halogen, cyano, —OR^{X1}, —N(R^{X1})₂, oxo, C₁-C₁₀ alkyl, or C₃-C₁₀ cycloalkyl; or two R^{X}, together with the atom they attach to, form C₃-C₁₀ cycloalkyl or 3- to 10-membered heterocycloalkyl;

each R^{X1} independent is H or C₁-C₆ alkyl;

R is C₆-C₁₀ aryl or 5- to 10-membered heteroaryl, wherein the C₆-C₁₀ aryl or 5- to 10-membered heteroaryl is optionally substituted with one or more halogen, cyano, —OR^{S}, —N(R^{S})₂, C₁-C₁₀ alkyl, or C₃-C₁₀ cycloalkyl; and

each R^{S} independent is H or C₁-C₆ alkyl.

In some embodiments, the compound is of Formula (II), (II-1), or (II-2):

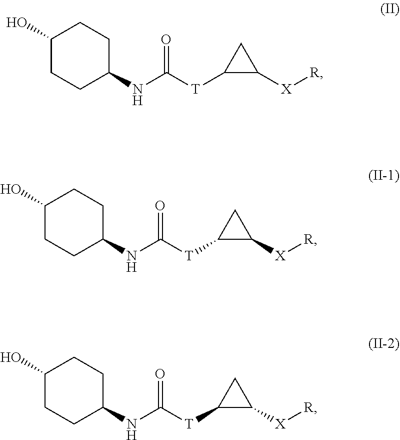

or a pharmaceutically acceptable salt thereof.

In some embodiments, the compound is of Formula (III):

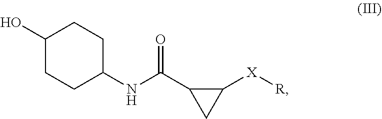

or a pharmaceutically acceptable salt thereof.

In some embodiments, the compound is of Formula (IV), (IV-1), or (IV-2):

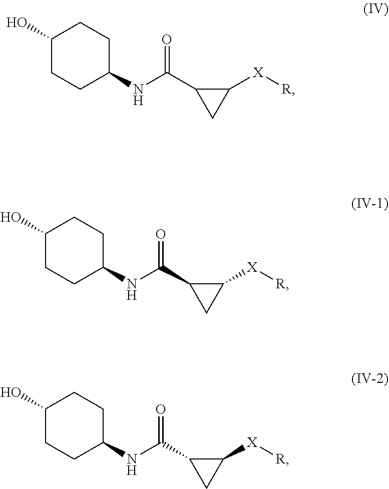

or a pharmaceutically acceptable salt thereof.

In some embodiments, T is absent.

In some embodiments, T is C₁-C₅ alkylene or 2- to 5-membered heteroalkylene, wherein the C₁-C₅ alkylene or 1- to 5-membered heteroalkylene is optionally substituted with one or more R^{T}.

In some embodiments, T is C₁-C₅ alkylene optionally substituted with one or more R^{T}.

In some embodiments, T is C₁-C₅ alkylene (e.g., CH₂, (CH₂)₂, (CH₂)₃, (CH₂)₄, or (CH₂)₅).

In some embodiments, T is C₁-C₅ alkylene substituted with one or more R^{T}.

In some embodiments, T is 2- to 5-membered heteroalkylene optionally substituted with one or more R^{T}.

In some embodiments, T is 2- to 5-membered heteroalkylene.

In some embodiments, T is 2- to 5-membered heteroalkylene including one heteroatom O. In some embodiments, T is —CH₂OCH₂CH₂CH₂—*, —CH₂CH₂OCH₂CH₂—*, —CH₂CH₂CH₂OCH₂—*, —CH₂OCH₂CH₂—*, —CH₂CH₂OCH₂—*, or —CH₂OCH₂—*, wherein * denotes attachment to cyclopropyl.

In some embodiments, T is 2- to 5-membered heteroalkylene including one heteroatom S. In some embodiments, T is —CH₂SCH₂CH₂CH₂—*, —CH₂CH₂SCH₂CH₂—*, —CH₂CH₂CH₂SCH₂—*, —CH₂SCH₂CH₂—*, —CH₂CH₂SCH₂—*, or —CH₂SCH₂—*, wherein * denotes attachment to cyclopropyl.

In some embodiments, T is 2- to 5-membered heteroalkylene including one heteroatom N. In some embodiments, T is —CH₂NCH₂CH₂CH₂—*, —CH₂CH₂NCH₂CH₂—*, —CH₂CH₂CH₂NCH₂—*, —CH₂NCH₂CH₂—*, —CH₂CH₂NCH₂—*, or —CH₂NCH₂—*, wherein * denotes attachment to cyclopropyl.

In some embodiments, T is 2- to 5-membered heteroalkylene substituted with one or more R^{T}.

In some embodiments, each R^{T} independent is halogen, cyano, —OR^{T1}, —N(R^{T1})₂, oxo, C₁-C₁₀ alkyl, or C₃-C₁₀ cycloalkyl.

In some embodiments, at least one R^{T} is halogen.

In some embodiments, at least one R^{T} is cyano.

In some embodiments, at least one R^{T} is —OR^{T1} (e.g., —OH or —O(C₁-C₁₀ alkyl)).

In some embodiments, at least one R^{T} is —N(R^{T1})₂ (e.g., —NH₂, —NH(C₁-C₁₀ alkyl), or —N(C₁-C₁₀ alkyl)₂).

In some embodiments, at least one R^{T} is oxo.

In some embodiments, at least one R^{T} is C₃-C₁₀ cycloalkyl.

In some embodiments, two R^{T}, together with the atom they attach to, form C₃-C₁₀ cycloalkyl or 3- to 10-membered heterocycloalkyl.

In some embodiments, two R^{T}, together with the atom they attach to, form C₃-C₁₀ cycloalkyl (e.g., C₃-C₆ cycloalkyl (e.g., cyclopropyl, cyclobutyl. cyclopentyl, or cyclohexyl)).

In some embodiments, two R^{T}, together with the atom they attach to, form 3- to 10-membered heterocycloalkyl (e.g., 4- to 6-membered heterocycloalkyl (e.g., tetrahydropyranyl)).

In some embodiments, at least one R^{T1} is H.

In some embodiments, each R^{T1} is H.

In some embodiments, at least one R^{T1} is C₁-C₆ alkyl.

In some embodiments, each R^{T1} is C₁-C₆ alkyl.

In some embodiments, X is C₂-C₅ alkylene optionally substituted with one or more R^{X}.

In some embodiments, X is C₂-C₅ alkylene (e.g., (CH₂)₂, (CH₂)₃, (CH₂)₄, or (CH₂)₅). In some embodiments, X is C₂-C₅ alkylene substituted with one or more R^{X}.

In some embodiments, X is 2- to 5-membered heteroalkylene optionally substituted with one or more R^{X}.

In some embodiments, X is 2- to 5-membered heteroalkylene including one heteroatom O. In some embodiments, X is —CH₂OCH₂CH₂CH₂—*, —CH₂CH₂OCH₂CH₂—*, —CH₂CH₂CH₂OCH₂—*, —CH₂OCH₂CH₂—*, —CH₂CH₂OCH₂—*, or —CH₂OCH₂—*, wherein * denotes attachment to R.

In some embodiments, X is 2- to 5-membered heteroalkylene including one heteroatom S. In some embodiments, X is —CH₂SCH₂CH₂CH₂—*, —CH₂CH₂SCH₂CH₂—*, —CH₂CH₂CH₂SCH₂—*, —CH₂SCH₂CH₂—*, —CH₂CH₂SCH₂—*, or —CH₂SCH₂—*, wherein * denotes attachment to R.

In some embodiments, X is 2- to 5-membered heteroalkylene including one heteroatom N. In some embodiments, X is —CH₂NCH₂CH₂CH₂—*, —CH₂CH₂NCH₂CH₂—*, —CH₂CH₂CH₂NCH₂—*, —CH₂NCH₂CH₂—*, —CH₂CH₂NCH₂—*, or —CH₂NCH₂—*, wherein * denotes attachment to R.

In some embodiments, X is 2- to 5-membered heteroalkylene substituted with one or more R^{X}.

In some embodiments, X is —CH₂SOCH₂CH₂CH₂—*, —CH₂CH₂SOCH₂CH₂—*, —CH₂CH₂CH₂SOCH₂—*, —CH₂SOCH₂CH₂—*, —CH₂CH₂SOCH₂—*, —CH₂SOCH₂—*, —CH₂SO₂CH₂CH₂CH₂—*, —CH₂CH₂SO₂CH₂CH₂—*, —CH₂CH₂CH₂SO₂CH₂—*, —CH₂SO₂CH₂CH₂—*, —CH₂CH₂SO₂CH₂—*, or —CH₂SO₂CH₂—*, wherein * denotes attachment to R.

In some embodiments, each R^{X} independent is halogen, cyano, —OR^{X1}, —N(R^{X1})₂, oxo, C₁-C₁₀ alkyl, or C₃-C₁₀ cycloalkyl.

In some embodiments, at least one R^{X} is halogen.

In some embodiments, at least one R^{X} is cyano.

In some embodiments, at least one R^{X} is —OR^{X1} (e.g., —OH or —O(C₁-C₁₀ alkyl)).

In some embodiments, at least one R^{X} is —N(R^{X1})₂ (e.g., —NH₂, —NH(C₁-C₁₀ alkyl), or —N(C₁-C₁₀ alkyl)₂).

In some embodiments, at least one R^{X} is oxo.

In some embodiments, at least one R^{X} is C₁-C₁₀ alkyl.

In some embodiments, at least one R^{X} is C₃-C₁₀ cycloalkyl.

In some embodiments, two R^{X}, together with the atom they attach to, form C₃-C₁₀ cycloalkyl or 3- to 10-membered heterocycloalkyl.

In some embodiments, two R^{X}, together with the atom they attach to, form C₃-C₁₀ cycloalkyl (e.g., C₃-C₆ cycloalkyl (e.g., cyclopropyl, cyclobutyl, cyclopentyl, or cyclohexyl)).

In some embodiments, two R^{X}, together with the atom they attach to, form 3- to 10-membered heterocycloalkyl (e.g., 4- to 6-membered heterocycloalkyl (e.g., tetrahydropyranyl)).

In some embodiments, at least one R^{X1} is H. In some embodiments, each R^{X1} is H.

In some embodiments, at least one R^{X1} is C₁-C₆ alkyl.

In some embodiments, each R^{X1} is C₁-C₆ alkyl.

In some embodiments, R is C₆-C₁₀ aryl optionally substituted with one or more halogen, cyano, —OR^{S}, —N(R^{S})₂, C₁-C₁₀ alkyl, or C₃-C₁₀ cycloalkyl.

In some embodiments, R is C₆-C₁₀ aryl.

In some embodiments, R is C₆-C₁₀ aryl substituted with one or more halogen, cyano, —OR^{S}, —N(R^{S})₂, C₁-C₁₀ alkyl, or C₃-C₁₀ cycloalkyl.

In some embodiments, R is phenyl optionally substituted with one or more halogen, cyano, —OR^{S}, —N(R^{S})₂, C₁-C₁₀ alkyl, or C₃-C₁₀ cycloalkyl.

In some embodiments, R is phenyl.

In some embodiments, R is phenyl substituted with one or more halogen, cyano, —OR^{S}, —N(R^{S})₂, C₁-C₁₀ alkyl, or C₃-C₁₀ cycloalkyl.

In some embodiments, R is 5- to 10-membered heteroaryl optionally substituted with one or more halogen, cyano, —OR^{S}, —N(R^{S})₂, C₁-C₁₀ alkyl, or C₃-C₁₀ cycloalkyl.

In some embodiments, R is 5- to 10-membered heteroaryl.

In some embodiments, R is 5- to 10-membered heteroaryl substituted with one or more halogen, cyano, —OR^{S}, —N(R^{S})₂, C₁-C₁₀ alkyl, or C₃-C₁₀ cycloalkyl.

In some embodiments, R is pyridyl, pyrazolyl, thiazolyl, oxazolyl, or imidazyolyl, wherein the pyridyl, pyrazolyl, thiazolyl, oxazolyl, or imidazyolyl is optionally substituted with one or more halogen, cyano, —OR^{S}, —N(R^{S})₂, C₁-C₁₀ alkyl, or C₃-C₁₀ cycloalkyl.

In some embodiments, R is pyridyl, pyrazolyl, thiazolyl, oxazolyl, or imidazyolyl.

In some embodiments, R is pyridyl, pyrazolyl, thiazolyl, oxazolyl, or imidazyolyl, wherein the pyridyl, pyrazolyl, thiazolyl, oxazolyl, or imidazyolyl is substituted with one or more halogen, cyano, —OR^{S}, —N(R^{S})₂, C₁-C₁₀ alkyl, or C₃-C₁₀ cycloalkyl.

In some embodiments, at least one R^{S} is H.

In some embodiments, each R^{S} is H.

In some embodiments, at least one R^{S} is C₁-C₆ alkyl.

In some embodiments, each R^{S} is C₁-C₆ alkyl.

In some embodiments, the compound is selected from:

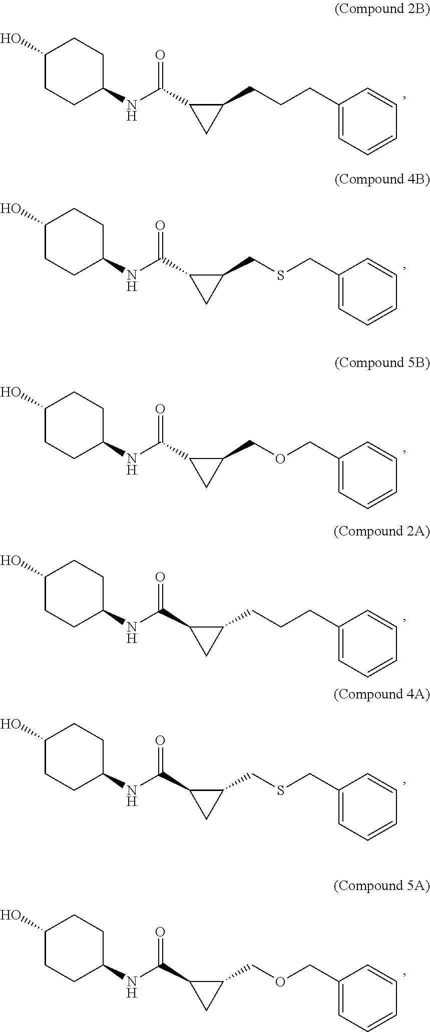

and pharmaceutically acceptable salts thereof.

In some embodiments, the compound is:

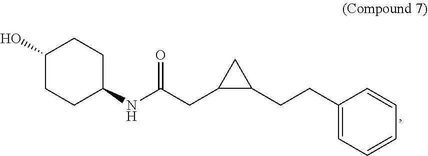 [SMILES: O=C(CC1CC1CCc1ccccc1)N[C@H]1CC[C@H](O)CC1]

or a pharmaceutically acceptable salt thereof.

It is understood that, advantageously, the trans-stereochemistry of the 4-hydroxycyclohexyl group and the (R,R)-stereochemistry of the cyclopropane ring may be established before assembling the mitofusin activators together. As such, the mitofusin activators may exhibit high stereoisomeric purity. In some embodiments, the compound is of greater than a 1:1 molar ratio of the (R,R) configuration relative to the (S,S) configuration of the cyclopropane ring. In some embodiments, the compound is of about 60% or greater (R,R) configuration, or about 70% or greater (R,R) configuration, or about 80% or greater (R,R) configuration, or about 90% or greater (R,R) configuration, or about 95% or greater (R,R) configuration, or about 97% or greater (R,R) configuration, or about 99% or greater (R,R) configuration, or about 99.9% or greater (R,R) configuration. In some embodiments, the compound is of an enantiomerically pure (R,R) configuration of the cyclopropane ring.

In some embodiments, the compound (e.g., Compound No. 2A, 2B, 4A, 4B, 5A, or 5B) is of about 10% enantiomeric excess (“ee”) or greater, about 20% ee or greater, about 30% ee or greater, about 40% ee or greater, about 50% ee or greater, about 60% ee or greater, about 70% ee or greater, about 80% ee or greater, about 90% ee or greater, about 95% ee or greater, about 96% ee or greater, about 97% ee or greater, about 98% ee or greater, about 99% ee or greater, about 99.5% ee or greater, or about 99.9% ee or greater.

In some aspects, the present disclosure provides a compound being an isotopic derivative (e.g., isotopically labeled compound) of any one of the compounds disclosed herein.

It is understood that the isotopic derivative can be prepared using any of a variety of art-recognized techniques. For example, the isotopic derivative can generally be prepared by carrying out the procedures disclosed in the Schemes and/or in the Examples described herein, by substituting an isotopically labeled reagent for a non-isotopically labeled reagent.

In some embodiments, the isotopic derivative is a deuterium labeled compound.

In some embodiments, the isotopic derivative is a deuterium labeled compound of any one of the compounds of the Formulae disclosed herein.

It is understood that the deuterium labeled compound comprises a deuterium atom having an abundance of deuterium that is substantially greater than the natural abundance of deuterium, which is 0.015%.

In some embodiments, the deuterium labeled compound has a deuterium enrichment factor for each deuterium atom of at least 3500 (52.5% deuterium incorporation at each deuterium atom), at least 4000 (60% deuterium incorporation), at least 4500 (67.5% deuterium incorporation), at least 5000 (75% deuterium), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation). As used herein, the term “deuterium enrichment factor” means the ratio between the deuterium abundance and the natural abundance of a deuterium.

It is understood that the deuterium labeled compound can be prepared using any of a variety of art-recognized techniques. For example, the deuterium labeled compound can generally be prepared by carrying out the procedures disclosed in the Schemes and/or in the Examples described herein, by substituting a deuterium labeled reagent for a non-deuterium labeled reagent.

A compound of the present disclosure or a pharmaceutically acceptable salt or solvate thereof that contains the aforementioned deuterium atom(s) is within the scope of the disclosure. Further, substitution with deuterium (i.e., ²H) may afford certain therapeutic advantages resulting from greater metabolic stability, e.g., increased in vivo half-life or reduced dosage requirements.

For the avoidance of doubt, it is to be understood that, where in this specification a group is qualified by “described herein”, the said group encompasses the first occurring and broadest definition as well as each and all of the particular definitions for that group.

A suitable pharmaceutically acceptable salt of a compound of the disclosure is, for example, an acid-addition salt of a compound of the disclosure which is sufficiently basic, for example, an acid-addition salt with, for example, an inorganic or organic acid, for example hydrochloric, hydrobromic, sulphuric, phosphoric, trifluoroacetic, formic, citric methane sulphonate or maleic acid. In addition, a suitable pharmaceutically acceptable salt of a compound of the disclosure which is sufficiently acidic is an alkali metal salt, for example a sodium or potassium salt, an alkaline earth metal salt, for example a calcium or magnesium salt, an ammonium salt or a salt with an organic base which affords a pharmaceutically acceptable cation, for example a salt with methylamine, dimethylamine, diethylamine, trimethylamine, piperidine, morpholine or tris-(2-hydroxyethyl)amine.

It will be understood that the compounds of the present disclosure and any pharmaceutically acceptable salts thereof, comprise stereoisomers, mixtures of stereoisomers, polymorphs of all isomeric forms of said compounds.

As used herein, the term “isomerism” means compounds that have identical molecular formulae but differ in the sequence of bonding of their atoms or in the arrangement of their atoms in space. Isomers that differ in the arrangement of their atoms in space are termed “stereoisomers.” Stereoisomers that are not mirror images of one another are termed “diastereoisomers,” and stereoisomers that are non-superimposable mirror images of each other are termed “enantiomers” or sometimes optical isomers. A mixture containing equal amounts of individual enantiomeric forms of opposite chirality is termed a “racemic mixture.”

As used herein, the term “chiral center” refers to a carbon atom bonded to four nonidentical substituents.

As used herein, the term “chiral isomer” means a compound with at least one chiral center. Compounds with more than one chiral center may exist either as an individual diastereomer or as a mixture of diastereomers, termed “diastereomeric mixture.” When one chiral center is present, a stereoisomer may be characterized by the absolute configuration (R or S) of that chiral center. Absolute configuration refers to the arrangement in space of the substituents attached to the chiral center. The substituents attached to the chiral center under consideration are ranked in accordance with the Sequence Rule of Cahn, Ingold and Prelog. (Cahn et al., Angew. Chem. Inter. Edit. 1966, 5, 385; errata 511; Cahn et al., Angew. Chem. 1966, 78, 413; Cahn and Ingold, J. Chem. Soc. 1951 (London), 612; Cahn et al., Experientia 1956, 12, 81; Cahn, J. Chem. Educ. 1964, 41, 116).

As used herein, the term “geometric isomer” means the diastereomers that owe their existence to hindered rotation about double bonds or a cycloalkyl linker (e.g., 1,3-cyclobutyl). These configurations are differentiated in their names by the prefixes cis and trans, or Z and E, which indicate that the groups are on the same or opposite side of the double bond in the molecule according to the Cahn-Ingold-Prelog rules.

It is to be understood that the compounds of the present disclosure may be depicted as different chiral isomers or geometric isomers. It is also to be understood that when compounds have chiral isomeric or geometric isomeric forms, all isomeric forms are intended to be included in the scope of the present disclosure, and the naming of the compounds does not exclude any isomeric forms, it being understood that not all isomers may have the same level of activity.

It is to be understood that the structures and other compounds discussed in this disclosure include all atropic isomers thereof. It is also to be understood that not all atropic isomers may have the same level of activity.

As used herein, the term “atropic isomers” are a type of stereoisomer in which the atoms of two isomers are arranged differently in space. Atropic isomers owe their existence to a restricted rotation caused by hindrance of rotation of large groups about a central bond. Such atropic isomers typically exist as a mixture, however as a result of recent advances in chromatography techniques, it has been possible to separate mixtures of two atropic isomers in select cases.

As used herein, the term “tautomer” is one of two or more structural isomers that exist in equilibrium and is readily converted from one isomeric form to another. This conversion results in the formal migration of a hydrogen atom accompanied by a switch of adjacent conjugated double bonds. Tautomers exist as a mixture of a tautomeric set in solution. In solutions where tautomerisation is possible, a chemical equilibrium of the tautomers will be reached. The exact ratio of the tautomers depends on several factors, including temperature, solvent and pH. The concept of tautomers that are interconvertible by tautomerisations is called tautomerism. Of the various types of tautomerism that are possible, two are commonly observed. In keto-enol tautomerism a simultaneous shift of electrons and a hydrogen atom occurs. Ring-chain tautomerism arises as a result of the aldehyde group (—CHO) in a sugar chain molecule reacting with one of the hydroxy groups (—OH) in the same molecule to give it a cyclic (ring-shaped) form as exhibited by glucose.

It is to be understood that the compounds of the present disclosure may be depicted as different tautomers. It should also be understood that when compounds have tautomeric forms, all tautomeric forms are intended to be included in the scope of the present disclosure, and the naming of the compounds does not exclude any tautomer form. It will be understood that certain tautomers may have a higher level of activity than others.

Compounds that have the same molecular formula but differ in the nature or sequence of bonding of their atoms or the arrangement of their atoms in space are termed “isomers”. Isomers that differ in the arrangement of their atoms in space are termed “stereoisomers”. Stereoisomers that are not mirror images of one another are termed “diastereomers” and those that are non-superimposable mirror images of each other are termed “enantiomers”. When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarised light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

The compounds of this disclosure may possess one or more asymmetric centers: such compounds can therefore be produced as individual (R)- or (S)-stereoisomers or as mixtures thereof. Unless indicated otherwise, the description or naming of a particular compound in the specification and claims is intended to include both individual enantiomers and mixtures, racemic or otherwise, thereof. The methods for the determination of stereochemistry and the separation of stereoisomers are well-known in the art (see discussion in Chapter 4 of “Advanced Organic Chemistry”, 4th edition J. March, John Wiley and Sons, New York, 2001), for example by synthesis from optically active starting materials or by resolution of a racemic form. Some of the compounds of the disclosure may have geometric isomeric centers (E- and Z-isomers). It is to be understood that the present disclosure encompasses all optical, diastereoisomers and geometric isomers and mixtures thereof that possess inflammasome inhibitory activity.

The present disclosure also encompasses compounds of the disclosure as defined herein which comprise one or more isotopic substitutions.

It is to be understood that the compounds of any Formula described herein include the compounds themselves, as well as their salts, and their solvates, if applicable. A salt, for example, can be formed between an anion and a positively charged group (e.g., amino) on a substituted compound disclosed herein. Suitable anions include chloride, bromide, iodide, sulphate, bisulphate, sulphamate, nitrate, phosphate, citrate, methanesulphonate, trifluoroacetate, glutamate, glucuronate, glutarate, malate, maleate, succinate, fumarate, tartrate, tosylate, salicylate, lactate, naphthalenesulphonate, and acetate (e.g., trifluoroacetate).

As used herein, the term “pharmaceutically acceptable anion” refers to an anion suitable for forming a pharmaceutically acceptable salt. Likewise, a salt can also be formed between a cation and a negatively charged group (e.g., carboxylate) on a substituted compound disclosed herein. Suitable cations include sodium ion, potassium ion, magnesium ion, calcium ion, and an ammonium cation such as tetramethylammonium ion or diethylamine ion. The substituted compounds disclosed herein also include those salts containing quaternary nitrogen atoms.

It is to be understood that the compounds of the present disclosure, for example, the salts of the compounds, can exist in either hydrated or unhydrated (the anhydrous) form or as solvates with other solvent molecules. Nonlimiting examples of hydrates include monohydrates, dihydrates, etc. Nonlimiting examples of solvates include ethanol solvates, acetone solvates, etc.

As used herein, the term “solvate” means solvent addition forms that contain either stoichiometric or non-stoichiometric amounts of solvent. Some compounds have a tendency to trap a fixed molar ratio of solvent molecules in the crystalline solid state, thus forming a solvate. If the solvent is water the solvate formed is a hydrate; and if the solvent is alcohol, the solvate formed is an alcoholate. Hydrates are formed by the combination of one or more molecules of water with one molecule of the substance in which the water retains its molecular state as H₂O.

As used herein, the term “analog” refers to a chemical compound that is structurally similar to another but differs slightly in composition (as in the replacement of one atom by an atom of a different element or in the presence of a particular functional group, or the replacement of one functional group by another functional group). Thus, an analog is a compound that is similar or comparable in function and appearance, but not in structure or origin to the reference compound.

As used herein, the term “derivative” refers to compounds that have a common core structure and are substituted with various groups as described herein.

As used herein, the term “bioisostere” refers to a compound resulting from the exchange of an atom or of a group of atoms with another, broadly similar, atom or group of atoms. The objective of a bioisosteric replacement is to create a new compound with similar biological properties to the parent compound. The bioisosteric replacement may be physicochemically or topologically based. Examples of carboxylic acid bioisosteres include, but are not limited to, acyl sulphonamides, tetrazoles, sulphonates and phosphonates. See. e.g., Patani and LaVoie, Chem. Rev. 96, 3147-3176, 1996.

It is also to be understood that certain compounds of the present disclosure may exist in solvated as well as unsolvated forms such as, for example, hydrated forms. A suitable pharmaceutically acceptable solvate is, for example, a hydrate such as hemi-hydrate, a mono-hydrate, a di-hydrate or a tri-hydrate.

### Synthesis of the Compounds

It is understood that the deuterium labeled compound can be prepared using any of a variety of art-recognized techniques. For example, the deuterium labeled compound can generally be prepared by carrying out the procedures disclosed in the Schemes and/or in the Examples described herein, by substituting a deuterium labeled reagent for a non-deuterium labeled reagent.

In some aspects, the present disclosure provides a method of preparing a compound disclosed herein.

In some aspects, the present disclosure provides a method of preparing a compound, comprising one or more steps as described herein.

In some aspects, the present disclosure provides a compound obtainable by, or obtained by, or directly obtained by a method for preparing a compound described herein.

In some aspects, the present disclosure provides an intermediate being suitable for use in a method for preparing a compound described herein.

In embodiments, a compound of described herein is prepared according to Scheme 1 below.

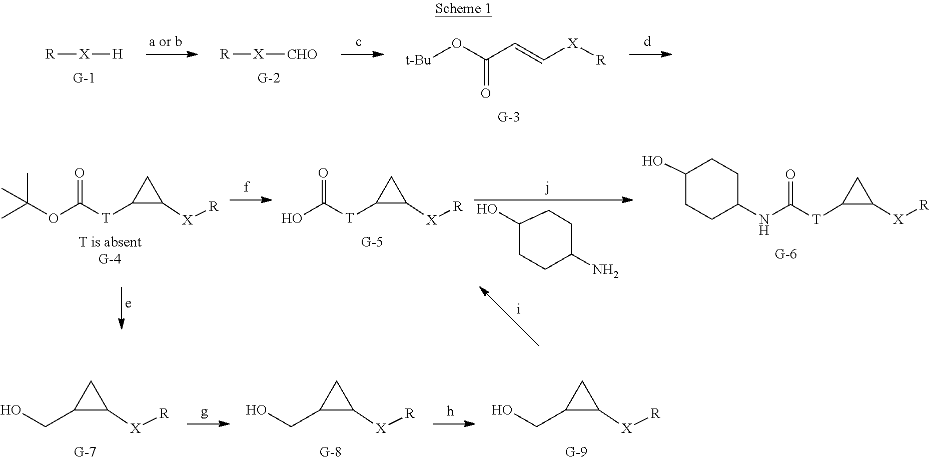

In some embodiments, the synthesis in Scheme 1 is performed with one or more of the following reagents and conditions:

Reagents and conditions:
(a) oxalyl chloride, dimethyl sulfoxide (DMSO), triethylamine (TEA), dichloromethane (DCM), −55-25° C., 20 min.
(b)
   (i) EtOH, EtONa, KI;
   (ii) 2-chloro-1,1-dimethoxyethane, 80° C., 12 h; H₂O, H₂SO₄, 60° C., 12 h.
(c) Tetrahydrofuran (THF), 20° C.
(d) NaH, DMSO, 20° C., 1.5 h.
(e) LiAH₄, THF, 0-25° C., 3 h.
(f) TFA, DCM, 25° C., 15 h.
(g) SOCl₂, TEA, CHCl₃, 0-70° C., 1 h.
(h) N(nBu)₄CN, THF, 70° C., 12 h.
(i) KOH, EtOH, H₂O, 100° C., 16 h.
(j) HOBt, N-ethyl-N′-(3-dimethylaminopropyl)carbodiimide hydrochloride (EDCI), N,N-diisopropylethylamine (DIEA), DMV, 25° C.

The compounds of the present disclosure can be prepared by any suitable technique known in the art. Particular processes for the preparation of these compounds are described further in the accompanying examples.

In the description of the synthetic methods described herein and in any referenced synthetic methods that are used to prepare the starting materials, it is to be understood that all proposed reaction conditions, including choice of solvent, reaction atmosphere, reaction temperature, duration of the experiment and workup procedures, can be selected by a person skilled in the art.

It is understood by one skilled in the art of organic synthesis that the functionality present on various portions of the molecule must be compatible with the reagents and reaction conditions utilized.

It will be appreciated that during the synthesis of the compounds of the disclosure in the processes defined herein, or during the synthesis of certain starting materials, it may be desirable to protect certain substituent groups to prevent their undesired reaction. The skilled chemist will appreciate when such protection is required, and how such protecting groups may be put in place, and later removed. For examples of protecting groups see one of the many general texts on the subject, for example, ‘Protective Groups in Organic Synthesis’ by Theodora Green (publisher: John Wiley & Sons). Protecting groups may be removed by any convenient method described in the literature or known to the skilled chemist as appropriate for the removal of the protecting group in question, such methods being chosen so as to effect removal of the protecting group with the minimum disturbance of groups elsewhere in the molecule. Thus, if reactants include, for example, groups such as amino, carboxy or hydroxy it may be desirable to protect the group in some of the reactions mentioned herein.

The resultant compounds of the present disclosure can be isolated and purified using techniques well known in the art.

Moreover, by utilizing the procedures described herein, in conjunction with ordinary skills in the art, additional compounds of the present disclosure can be readily prepared. Those skilled in the art will readily understand that known variations of the conditions and processes of the following preparative procedures can be used to prepare these compounds.

As will be understood by the person skilled in the art of organic synthesis, compounds of the present disclosure are readily accessible by various synthetic routes, some of which are exemplified in the accompanying examples. The skilled person will easily recognize which kind of reagents and reactions conditions are to be used and how they are to be applied and adapted in any particular instance—wherever necessary or useful—in order to obtain the compounds of the present disclosure. Furthermore, some of the compounds of the present disclosure can readily be synthesized by reacting other compounds of the present disclosure under suitable conditions, for instance, by converting one particular functional group being present in a compound of the present disclosure, or a suitable precursor molecule thereof, into another one by applying standard synthetic methods, like reduction, oxidation, addition or substitution reactions; those methods are well known to the skilled person. Likewise, the skilled person will apply—whenever necessary or useful—synthetic protecting (or protective) groups; suitable protecting groups as well as methods for introducing and removing them are well-known to the person skilled in the art of chemical synthesis and are described, in more detail, in, e.g., P.G.M. Wuts, T.W. Greene, “Greene's Protective Groups in Organic Synthesis”, 4th edition (2006) (John Wiley & Sons).

### Biological Assays

Compounds designed, selected and/or optimized by methods described above, once produced, can be characterized using a variety of assays known to those skilled in the art to determine whether the compounds have biological activity. For example, the molecules can be characterized by conventional assays, including but not limited to those assays described below, to determine whether they have a predicted activity, binding activity and/or binding specificity.

Furthermore, high-throughput screening can be used to speed up analysis using such assays. As a result, it can be possible to rapidly screen the molecules described herein for activity, using techniques known in the art. General methodologies for performing high-throughput screening are described, for example, in Devlin (1998) High Throughput Screening, Marcel Dekker; and U.S. Pat. No. 5,763,263. High-throughput assays can use one or more different assay techniques including, but not limited to, those described below.

In some embodiments, the biological assay involves evaluation of the dose-response of a compound of described herein, e.g., in Mfn1- or Mfn2-deficient cells.

In some embodiments, the biological assay involves evaluation of Mitofusin-stimulating activities of a compound of described herein, e.g., in Mfn1-null or Mfn2-null cells.

In some embodiments, the biological assay was performed with wild-type MEFs (e.g., prepared from E10.5 c57/b16 mouse embryos).

In some embodiments, the biological assay was performed with SV-40 T antigen-immortalized MFN1 null (CRL-2992), MFN2 null (CRL-2993), and/or MFN1/MFN2 double null MEFs (CRL-2994).
In some embodiments, the biological assay involves evaluation of in vitro stability, e.g., in human and mouse liver microsomes.
In some embodiments, the biological assay involves parallel artificial membrane permeability assay (PAMPA)
In some embodiments, the PAMPA is performed with PVDF membrane, e.g., pre-coated with 5 μL of 1% brain polar lipid extract (porcine)/dodecane mixture.

### Pharmaceutical Compositions

In another exemplary aspect, the disclosure features pharmaceutical compositions comprising any compound herein, or a pharmaceutically acceptable form thereof. In some embodiments, a pharmaceutical composition comprises a therapeutically effective amount of any compound described herein, or any pharmaceutically acceptable form thereof.

In some embodiments, a pharmaceutically acceptable form of a compound includes any pharmaceutically acceptable salts, hydrates, solvates, isomers, prodrugs, and isotopically labeled derivatives thereof.

In some embodiments, a pharmaceutical composition comprises any compound described herein, or a pharmaceutically acceptable salt thereof.

In some embodiments, a pharmaceutical composition comprises a pharmaceutically acceptable excipient.

For the purposes of the present invention the term “excipient” and “carrier” are used interchangeably throughout the description of the present invention and said terms are defined herein as, “ingredients which are used in the practice of formulating a safe and effective pharmaceutical composition.”

The formulator will understand that excipients are used primarily to serve in delivering a safe, stable, and functional pharmaceutical, serving not only as part of the overall vehicle for delivery but also as a means for achieving effective absorption by the recipient of the active ingredient. An excipient may fill a role as simple and direct as being an inert filler, or an excipient as used herein may be part of a pH stabilizing system or coating to insure delivery of the ingredients safely to the stomach. The formulator can also take advantage of the fact the compounds of the present invention have improved cellular potency, pharmacokinetic properties, as well as improved oral bioavailability.

Accordingly, in some embodiments, provided herein are pharmaceutical compositions comprising one or more compounds as disclosed herein, or a pharmaceutically acceptable form thereof (e.g., pharmaceutically acceptable salts, hydrates, solvates, isomers, prodrugs, and isotopically labeled derivatives), and one or more pharmaceutically acceptable excipients, carriers, including inert solid diluents and fillers, diluents, including sterile aqueous solution and various organic solvents, permeation enhancers, solubilizers and adjuvants. In some embodiments, a pharmaceutical composition described herein includes a second active agent such as an additional therapeutic agent, (e.g., a chemotherapeutic).

Accordingly, the present teachings also provide pharmaceutical compositions that include at least one compound described herein, or any pharmaceutically salt thereof, and one or more pharmaceutically acceptable carriers, excipients, or diluents. Examples of such carriers are well known to those skilled in the art and can be prepared in accordance with acceptable pharmaceutical procedures, such as, for example, those described in Remington's Pharmaceutical Sciences, 17th edition, ed. Alfonoso R. Gennaro, Mack Publishing Company, Easton, PA (1985), the entire disclosure of which is incorporated by reference herein for all purposes. As used herein, “pharmaceutically acceptable” refers to a substance that is acceptable for use in pharmaceutical applications from a toxicological perspective and does not adversely interact with the active ingredient. Accordingly, pharmaceutically acceptable carriers are those that are compatible with the other ingredients in the composition and are biologically acceptable. Supplementary active ingredients can also be incorporated into the pharmaceutical compositions.

Compounds of the present teachings can be administered orally or parenterally, neat or in combination with conventional pharmaceutical carriers. Applicable solid carriers can include one or more substances which can also act as flavoring agents, lubricants, solubilizers, suspending agents, fillers, glidants, compression aids, binders or tablet-disintegrating agents, or encapsulating materials. Pharmaceutical compositions in the form of oral formulations containing a compound disclosed herein can comprise any conventionally used oral form, including tablets, capsules, buccal forms, troches, lozenges and oral liquids, suspensions or solutions. In powders, the carrier can be a finely divided solid, which is an admixture with a finely divided compound. In tablets, a compound disclosed herein can be mixed with a carrier having the necessary compression properties in suitable proportions and compacted in the shape and size desired. The powders and tablets can contain up to 99% of the compound.

Capsules can contain mixtures of one or more compound(s) disclosed herein with inert filler(s) and/or diluent(s) such as pharmaceutically acceptable starches (e.g. , corn, potato or tapioca starch), sugars, artificial sweetening agents, powdered celluloses (e.g., crystalline and microcrystalline celluloses), flours, gelatins, gums, and the like.

Useful tablet formulations can be made by conventional compression, wet granulation or dry granulation methods and utilize pharmaceutically acceptable diluents, binding agents, lubricants, disintegrants, surface modifying agents (including surfactants), suspending or stabilizing agents, including, but not limited to, magnesium stearate, stearic acid, sodium lauryl sulfate, talc, sugars, lactose, dextrin, starch, gelatin, cellulose, methyl cellulose, microcrystalline cellulose, sodium carboxymethyl cellulose, carboxymethylcellulose calcium, polyvinylpyrrolidine, alginic acid, acacia gum, xanthan gum, sodium citrate, complex silicates, calcium carbonate, glycine, sucrose, sorbitol, dicalcium phosphate, calcium sulfate, lactose, kaolin, mannitol, sodium chloride, low melting waxes, and ion exchange resins. Surface modifying agents include nonionic and anionic surface modifying agents. Representative examples of surface modifying agents include, but are not limited to, poloxamer 188, benzalkonium chloride, calcium stearate, cetostearl alcohol, cetomacrogol emulsifying wax, sorbitan esters, colloidal silicon dioxide, phosphates, sodium dodecylsulfate, magnesium aluminum silicate, and triethanolamine. Oral formulations described herein can utilize standard delay or time-release formulations to alter the absorption of the compound(s). An oral formulation can also consist of administering a compound disclosed herein in water or fruit juice, containing appropriate solubilizers or emulsifiers as needed.

Liquid carriers can be used in preparing solutions, suspensions, emulsions, syrups, elixirs, and for inhaled delivery. A compound of the present teachings can be dissolved or suspended in a pharmaceutically acceptable liquid carrier such as water, an organic solvent, or a mixture of both, or a pharmaceutically acceptable oils or fats. The liquid carrier can contain other suitable pharmaceutical additives such as solubilizers, emulsifiers, buffers, preservatives, sweeteners, flavoring agents, suspending agents, thickening agents, colors, viscosity regulators, stabilizers, and osmo-regulators. Examples of liquid carriers for oral and parenteral administration include, but are not limited to, water (particularly containing additives as described herein, e.g., cellulose derivatives such as a sodium carboxymethyl cellulose solution), alcohols (including monohydric alcohols and polyhydric alcohols, e.g., glycols) and their derivatives, and oils (e.g., fractionated coconut oil and arachis oil). For parenteral administration, the carrier can be an oily ester such as ethyl oleate and isopropyl myristate. Sterile liquid carriers are used in sterile liquid form compositions for parenteral administration. The liquid carrier for pressurized compositions can be halogenated hydrocarbon or other pharmaceutically acceptable propellants.

Liquid pharmaceutical compositions, which are sterile solutions or suspensions, can be utilized by, for example, intramuscular, intraperitoneal or subcutaneous injection. Sterile solutions can also be administered intravenously. Compositions for oral administration can be in either liquid or solid form.

In some embodiments, a pharmaceutical composition is in unit dosage form, for example, as tablets, capsules, powders, solutions, suspensions, emulsions, granules, or suppositories. In such form, the pharmaceutical composition can be sub-divided in unit dose(s) containing appropriate quantities of the compound. The unit dosage forms can be packaged compositions, for example, packeted powders, vials, ampoules, prefilled syringes or sachets containing liquids. Alternatively, the unit dosage form can be a capsule or tablet itself, or it can be the appropriate number of any such compositions in package form. Such unit dosage form can contain from about 1 mg/kg of compound to about 500 mg/kg of compound, and can be given in a single dose or in two or more doses. Such doses can be administered in any manner useful in directing the compound(s) to the recipient's bloodstream, including orally, via implants, parenterally (including intravenous, intraperitoneal and subcutaneous injections), rectally, vaginally, and transdermally.

When administered for the treatment or inhibition of a particular disease state or disorder, it is understood that an effective dosage can vary depending upon the particular compound utilized, the mode of administration, and severity of the condition being treated, as well as the various physical factors related to the individual being treated. In therapeutic applications, a compound of the present teachings can be provided to a patient already suffering from a disease in an amount sufficient to cure or at least partially ameliorate the symptoms of the disease and its complications. The dosage to be used in the treatment of a specific individual typically must be subjectively determined by the attending physician. The variables involved include the specific condition and its state as well as the size, age and response pattern of the patient.

In some cases it may be desirable to administer a compound directly to the airways of the patient, using devices such as, but not limited to, metered dose inhalers, breath-operated inhalers, multidose dry-powder inhalers, pumps, squeeze-actuated nebulized spray dispensers, aerosol dispensers, and aerosol nebulizers. For administration by intranasal or intrabronchial inhalation, the compounds of the present teachings can be formulated into a liquid composition, a solid composition, or an aerosol composition. The liquid composition can include, by way of illustration, one or more compounds of the present teachings dissolved, partially dissolved, or suspended in one or more pharmaceutically acceptable solvents and can be administered by, for example, a pump or a squeeze-actuated nebulized spray dispenser. The solvents can be, for example, isotonic saline or bacteriostatic water. The solid composition can be, by way of illustration, a powder preparation including one or more compounds of the present teachings intermixed with lactose or other inert powders that are acceptable for intrabronchial use, and can be administered by, for example, an aerosol dispenser or a device that breaks or punctures a capsule encasing the solid composition and delivers the solid composition for inhalation. The aerosol composition can include, by way of illustration, one or more compounds of the present teachings, propellants, surfactants, and co-solvents, and can be administered by, for example, a metered device. The propellants can be a chlorofluorocarbon (CFC), a hydrofluoroalkane (HFA), or other propellants that are physiologically and environmentally acceptable.]

Compounds described herein can be administered parenterally or intraperitoneally. Solutions or suspensions of these compounds or a pharmaceutically acceptable salts, hydrates, or esters thereof can be prepared in water suitably mixed with a surfactant such as hydroxyl-propylcellulose. Dispersions can also be prepared in glycerol, liquid polyethylene glycols, and mixtures thereof in oils. Under ordinary conditions of storage and use, these preparations typically contain a preservative to inhibit the growth of microorganisms.

The pharmaceutical forms suitable for injection can include sterile aqueous solutions or dispersions and sterile powders for the extemporaneous preparation of sterile injectable solutions or dispersions. In some embodiments, the form can sterile and its viscosity permits it to flow through a syringe. The form preferably is stable under the conditions of manufacture and storage and can be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), suitable mixtures thereof, and vegetable oils.

Compounds described herein can be administered transdermally. i.e., administered across the surface of the body and the inner linings of bodily passages including epithelial and mucosal tissues. Such administration can be carried out using the compounds of the present teachings including pharmaceutically acceptable salts, hydrates, or esters thereof, in lotions, creams, foams, patches, suspensions, solutions, and suppositories (rectal and vaginal).

Transdermal administration can be accomplished through the use of a transdermal patch containing a compound, such as a compound disclosed herein, and a carrier that can be inert to the compound, can be non-toxic to the skin, and can allow delivery of the compound for systemic absorption into the blood stream via the skin. The carrier can take any number of forms such as creams and ointments, pastes, gels, and occlusive devices. The creams and ointments can be viscous liquid or semisolid emulsions of either the oil-in-water or water-in-oil type. Pastes comprised of absorptive powders dispersed in petroleum or hydrophilic petroleum containing the compound can also be suitable. A variety of occlusive devices can be used to release the compound into the blood stream, such as a semi-permeable membrane covering a reservoir containing the compound with or without a carrier, or a matrix containing the compound. Other occlusive devices are known in the literature.

Compounds described herein can be administered rectally or vaginally in the form of a conventional suppository. Suppository formulations can be made from traditional materials, including cocoa butter, with or without the addition of waxes to alter the suppository's melting point, and glycerin. Water-soluble suppository bases, such as polyethylene glycols of various molecular weights, can also be used.

Lipid formulations or nanocapsules can be used to introduce compounds of the present teachings into host cells either in vitro or in vivo. Lipid formulations and nanocapsules can be prepared by methods known in the art.

To increase the effectiveness of compounds of the present teachings, it can be desirable to combine a compound with other agents effective in the treatment of the target disease. For example, other active compounds (i.e., other active ingredients or agents) effective in treating the target disease can be administered with compounds of the present teachings. The other agents can be administered at the same time or at different times than the compounds disclosed herein.

### Kits

In some embodiments, provided herein are kits. The kits can include a compound or pharmaceutically acceptable form thereof, or pharmaceutical composition as described herein, in suitable packaging, and written material that can include instructions for use, discussion of clinical studies, listing of side effects, and the like. Kits are well suited for the delivery of solid oral dosage forms such as tablets or capsules. Such kits can also include information, such as scientific literature references, package insert materials, clinical trial results, and/or summaries of these and the like, which indicate or establish the activities and/or advantages of the pharmaceutical composition, and/or which describe dosing, administration, side effects, drug interactions, or other information useful to the health care provider. Such information can be based on the results of various studies, for example, studies using experimental animals involving in vivo models and studies based on human clinical trials.

### Methods of Use

Compounds or pharmaceutical composition of the present teachings can be useful for the treatment or prevention of a disease, disorder, or condition in a subject, for example, a human subject. The present teachings accordingly provide methods of treating or preventing a disease, disorder, or condition in a subject by providing to a subject a compound of the present teachings (including its pharmaceutically acceptable salt) or a pharmaceutical composition that includes one or more compounds of the present teachings in combination or association with pharmaceutically acceptable carriers. Compounds of the present teachings can be administered alone or in combination with other therapeutically effective compounds or therapies for the treatment or prevention of a disease, disorder, or condition.

In some aspects, the present disclosure features a method of treating diseases, disorders, or conditions, comprising administering to a subject in need thereof any compound described herein in a pharmaceutical composition.

In some aspects, the present disclosure features any compound described herein in a pharmaceutical composition for use for treating diseases, disorders, or conditions, comprising administering to a subject in need thereof.

In some aspects, the present disclosure features use of any compound described herein in a pharmaceutical composition in the manufacture of a medicament for treating diseases, disorders, or conditions, comprising administering to a subject in need thereof.

In some aspects, the present disclosure features a method of activating mitofusin in a subject, comprising administering the compound or the pharmaceutical composition of any one of the preceding claims.

In some aspects, the present disclosure features any compound described herein in a pharmaceutical composition for use in activating mitofusin in a subject.

In some aspects, the present disclosure features use of the any compound described herein in a pharmaceutical composition in the manufacture of a medicament for activating mitofusin in a subject.

In some embodiments, a compound described herein, or any pharmaceutically acceptable form thereof such as a pharmaceutically acceptable salt thereof, can be used to treat or prevent a disease, disorder, or condition in a subject.

In some embodiments, a therapeutically effective amount of the compound or the pharmaceutical composition described herein is administered to the subject.

In some embodiments, the disease, disorder, or condition is associated with mitochondria.

In some embodiments, the disease, disorder, or condition is peripheral nervous system (PNS), central nervous system (CNS) genetic or non-genetic disorder, physical damage, or chemical injury.

In some embodiments, the PNS or CNS disorder is one or more conditions selected from the group consisting of a chronic neurodegenerative condition in which mitochondrial fusion, fitness, and/or trafficking is/are impaired; a disease or disorder associated with mitofusin 1 (MFN1) or mitofusin 2 (MFN2) dysfunction; a disease associated with mitochondrial fragmentation, dysfunction, and/or dysmotility; a degenerative neuromuscular condition; Charcot-Marie-Tooth disease; Amyotrophic Lateral Sclerosis; Huntington's disease; Alzheimer's disease; Parkinson's disease; hereditary motor and sensory neuropathy; autism; autosomal dominant optic atrophy (ADOA); muscular dystrophy; Lou Gehrig's disease; cancer; mitochondrial myopathy; diabetes mellitus and deafness (DAD); Leber's hereditary optic neuropathy (LHON); Leigh syndrome; subacute sclerosing encephalopathy; neuropathy, ataxia, retinitis pigmentosa, and ptosis (NARP); myoneurogenic gastrointestinal encephalopathy (MNGIE); myoclonic epilepsy with ragged red fibers (MERRF); mitochondrial myopathy, encephalomyopathy, lactic acidosis, and stroke-like symptoms (MELAS); mtDNA depletion; mitochondrial neurogastrointestinal encephalomyopathy (MNGIE); dysautonomic mitochondrial myopathy; mitochondrial channelopathy; pyruvate dehydrogenase complex deficiency (PDCD/PDH); diabetic neuropathy: chemotherapy-induced peripheral neuropathy; crush injury; spinal cord injury (SCI); traumatic brain injury; stroke; optic nerve injury; conditions that involve axonal disconnection; and any combination thereof.

In some embodiments, the subject is human.

In some embodiments, a compound described herein, or any pharmaceutically acceptable form thereof such as a pharmaceutically acceptable salt thereof, can be used to active mitofusin in a subject (e.g., human).

### Exemplary Embodiments

Exemplary Embodiment No. 1: A composition comprising: a mitofusin activator having a structure represented by

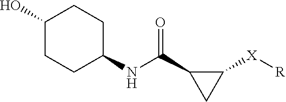

or a pharmaceutically acceptable salt thereof; wherein X is a 3-atom spacer group, and R is phenyl or substituted phenyl.

Exemplary Embodiment No. 2: The composition of claim 1, wherein X is —CH₂YCH₂— or —CH₂CH₂Y —; wherein Y is O, S, SO, SO₂, CR¹R², or NR³; wherein R¹ and R² are independently selected from the group consisting of H, F, C₁-C₁₀ alkyl, and C₃-C₁₀ cycloalkyl, or R¹ and R2 taken together form a cycloalkyl or heterocycloalkyl; and R³ is H, C₁-C₁₀ alkyl, or C₃-C₁₀ cycloalkyl.

Exemplary Embodiment No. 3: The composition of claim 2, wherein X is —CH₂YCH₂—.

Exemplary Embodiment No. 4: The composition of claim 3, wherein Y is O, S or CH₂.

Exemplary Embodiment No. 5: The composition of claim 1, wherein X is —(CH₂)₃—.

Exemplary Embodiment No. 6: The composition of claim 5, wherein the mitofusin activator has a structure represented by

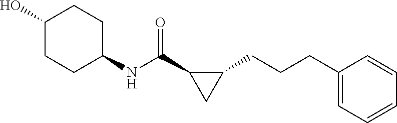 [SMILES: O=C(N[C@H]1CC[C@H](O)CC1)[C@@H]1C[C@H]1CCCc1ccccc1]

(1R,2R)-N-((1r,4R)-4-hydroxycyclohexyl)-2-(3-phenylpropyl)cyclopropane-1-carboxamide

Exemplary Embodiment No. 7: The composition of claim 6, wherein the mitofusin activator is at least partially crystalline.

Exemplary Embodiment No. 8: The composition of claim 1, wherein the mitofusin activator has a structure represented by one or more formulas selected from the group consisting of

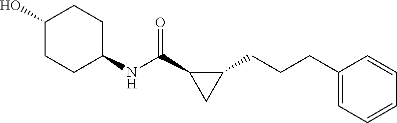 [SMILES: O=C(N[C@H]1CC[C@H](O)CC1)[C@@H]1C[C@H]1CCCc1ccccc1]

(1R,2R)-N-((1r,4R)-4-hydroxycyclohexyl)-2-(3-phenylpropyl)cyclopropane-1-carboxamide,

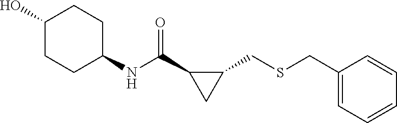 [SMILES: O=C(N[C@H]1CC[C@H](O)CC1)[C@@H]1C[C@H]1CSCc1ccccc1]

(1R,2R)-2-((benzylthio)methyl)-N-((1r,4R)-4-hydroxycyclohexyl)cyclopropane-1-carboxamide, and

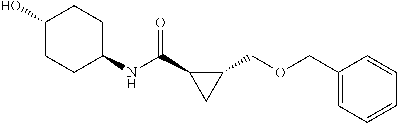 [SMILES: O=C(N[C@H]1CC[C@H](O)CC1)[C@@H]1C[C@H]1COCc1ccccc1]

(1R,2R)-2-((benzyloxy)methyl)-N-((1r,4R)-4-hydroxycyclohexyl)cyclopropane-1-carboxamide.

Exemplary Embodiment No. 9: The composition of claim 8, wherein the mitofusin activator is at least partially crystalline.

Exemplary Embodiment No. 10: The composition of claim 1, further comprising: a pharmaceutically acceptable excipient.

Exemplary Embodiment No. 11: An at least partially crystalline compound having a structure represented by

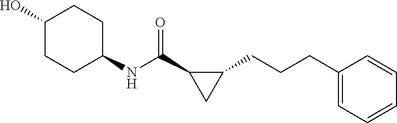 [SMILES: O=C(N[C@H]1CC[C@H](O)CC1)[C@@H]1C[C@H]1CCCc1ccccc1]

(1R,2R)-N-((1r,4R)-4-hydroxycyclohexyl)-2-(3-phenylpropyl)cyclopropane-1-carboxamide; or

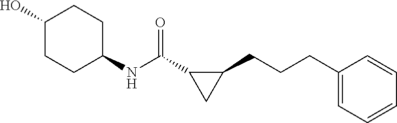

(1S,2S)-N-((1r,4R)-4-hydroxycyclohexyl)-2-(3-phenylpropyl)cyclopropane-1-carboxamide.

Exemplary Embodiment No. 12: A method comprising: administering a therapeutically effective amount of a composition comprising a mitofusin activator or a pharmaceutically acceptable salt thereof to a subject having or suspected of having a mitochondria-associated disease, disorder, or condition, the mitofusin activator having a structure represented by

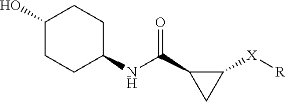

wherein X is a 3-atom spacer group, and R is phenyl or substituted phenyl.

Exemplary Embodiment No. 13: The method of claim 12, wherein X is —CH₂YCH₂— or —CH₂CH₂Y—; wherein Y is O, S, SO, SO₂, CR¹R², or NR³; wherein R¹ and R2 are independently selected from the group consisting of H, F, C₁-C₁₀ alkyl, and C₃-C₁₀ cycloalkyl, or R¹ and R² taken together form a cycloalkyl or heterocycloalkyl; and R³ is H, C₁-C₁₀ alkyl, or C₃-C₁₀ cycloalkyl.

Exemplary Embodiment No. 14: The method of claim 13, wherein X is —CH₂YCH₂—.

Exemplary Embodiment No. 15: The method of claim 14, wherein Y is O, S or CH₂.

Exemplary Embodiment No. 16: The method of claim 12, wherein X is —(CH₂)₃—.

Exemplary Embodiment No. 17: The method of claim 16, wherein the mitofusin activator has a structure represented by

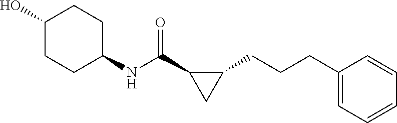 [SMILES: O=C(N[C@H]1CC[C@H](O)CC1)[C@@H]1C[C@H]1CCCc1ccccc1]

(1R,2R)-N-((1r,4R)-4-hydroxycyclohexyl)-2-(3-phenylpropyl)cyclopropane-1-carboxamide.

Exemplary Embodiment No. 18: The method of claim 17, wherein the mitofusin activator is at least partially crystalline.

Exemplary Embodiment No. 19: The method of claim 12, wherein the mitochondria-associated disease, disorder or condition is a peripheral nervous system (PNS) or central nervous system (CNS) genetic or non-genetic disorder, physical damage, and/or chemical injury.

Exemplary Embodiment No. 20: The method of claim 19, wherein the PNS or CNS disorder is one or more conditions selected from the group consisting of a chronic neurodegenerative condition in which mitochondrial fusion, fitness, and/or trafficking is/are impaired; a disease or disorder associated with mitofusin 1 (MFN1) or mitofusin 2 (MFN2) dysfunction; a disease associated with mitochondrial fragmentation, dysfunction, and/or dysmotility; a degenerative neuromuscular condition; Charcot-Marie-Tooth disease; Amyotrophic Lateral Sclerosis; Huntington's disease; Alzheimer's disease; Parkinson's disease; hereditary motor and sensory neuropathy; autism; autosomal dominant optic atrophy (ADOA); muscular dystrophy; Lou Gehrig's disease; cancer; mitochondrial myopathy; diabetes mellitus and deafness (DAD); Leber's hereditary optic neuropathy (LHON); Leigh syndrome; subacute sclerosing encephalopathy; neuropathy, ataxia, retinitis pigmentosa, and ptosis (NARP); myoneurogenic gastrointestinal encephalopathy (MNGIE); myoclonic epilepsy with ragged red fibers (MERRF); mitochondrial myopathy, encephalomyopathy, lactic acidosis, and stroke-like symptoms (MELAS); mtDNA depletion; mitochondrial neurogastrointestinal encephalomyopathy (MNGIE); dysautonomic mitochondrial myopathy; mitochondrial channelopathy; pyruvate dehydrogenase complex deficiency (PDCD/PDH); diabetic neuropathy; chemotherapy-induced peripheral neuropathy; crush injury; spinal cord injury (SCI); traumatic brain injury; stroke; optic nerve injury; conditions that involve axonal disconnection; and any combination thereof.

### Definitions

Unless stated otherwise, the following terms and phrases as used herein are intended to have the following meanings.

The terms “treat” or “treatment”, unless otherwise indicated by context, refer to any administration of a therapeutic molecule (e.g., any compound described herein) that partially or completely alleviates, ameliorates, relieves, inhibits, reduces severity of and/or reduces incidence of one or more symptoms or features of a particular disease, disorder, and/or condition (e.g., cancer).

As used herein, the term “preventing,” “prevent,” or “protecting against” describes delaying onset or slowing progression of a disease, condition or disorder.

As used herein, the term “subject” includes human and non-human animals, as well as cell lines, cell cultures, tissues, and organs. In some embodiments, the subject is a mammal. The mammal can be e.g., a human or appropriate non-human mammal, such as primate, mouse, rat, dog, cat, cow, horse, goat, camel, sheep or a pig. The subject can also be a bird or fowl. In some embodiments, the subject is a human.

As used herein, the term “subject in need thereof” refers to a subject having a disease or having an increased risk of developing the disease. A subject in need thereof can be one who has been previously diagnosed or identified as having a disease or disorder disclosed herein. A subject in need thereof can also be one who is suffering from a disease or disorder disclosed herein. Alternatively, a subject in need thereof can be one who has an increased risk of developing such disease or disorder relative to the population at large (i.e., a subject who is predisposed to developing such disorder relative to the population at large). A subject in need thereof can have a refractory or resistant a disease or disorder disclosed herein (i.e., a disease or disorder disclosed herein that does not respond or has not yet responded to treatment). The subject may be resistant at start of treatment or may become resistant during treatment. In some embodiments, the subject in need thereof received and failed all known effective therapies for a disease or disorder disclosed herein. In some embodiments, the subject in need thereof received at least one prior therapy.

The term “therapeutically effective amount” or “effective amount” refers to an amount of a conjugate effective to treat or prevent a disease or disorder in a subject (e.g., as described herein).

As used herein, the term “pharmaceutical composition” refers to a composition in which an active agent is formulated together with one or more pharmaceutically acceptable carriers. In some embodiments, the active agent is present in unit dose amount appropriate for administration in a therapeutic regimen that shows a statistically significant probability of achieving a predetermined therapeutic effect when administered to a relevant population. In some embodiments, a pharmaceutical composition may be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue: parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream, or foam; sublingually; ocularly; transdermally; or nasally, pulmonary, and to other mucosal surfaces.

As used herein, the term “administration” typically refers to the administration of a composition to a subject or system to achieve delivery of an agent that is, or is included in, the composition. Those of ordinary skill in the art will be aware of a variety of routes that may, in appropriate circumstances, be utilized for administration to a subject, for example a human. Examples of routes of administration include parenteral, e.g., intravenous, intradermal, subcutaneous, oral (e.g., inhalation), transdermal (i.e., topical), transmucosal, and rectal administration. For example, in some embodiments, administration may be ocular, oral, parenteral, topical, etc. In some embodiments, administration is parenteral (e.g., intravenous administration). In some embodiments, intravenous administration is intravenous infusion. In some particular embodiments, administration may be bronchial (e.g., by bronchial instillation), buccal, dermal (which may be or comprise, for example, one or more of topical to the dermis, intradermal, interdermal, transdermal, etc.), enteral, intra-arterial, intradermal, intragastric, intramedullary, intramuscular, intranasal, intraperitoneal, intrathecal, intravenous, intraventricular, within a specific organ (e. g. intrahepatic), mucosal, nasal, oral, rectal, subcutaneous, sublingual, topical, tracheal (e.g., by intratracheal instillation), vaginal, vitreal, etc.

Unless otherwise indicated, the term “alkyl” by itself or as part of another term refers to a substituted or straight chain or branched, saturated or unsaturated hydrocarbon having the indicated number of carbon atoms (e.g., “C₁-C₅ alkyl” or “C₁-C₁₀” alkyl refer to an alkyl group having from 1 to 8 or 1 to 10 carbon atoms, respectively). When the number of carbon atoms is not indicated, the alkyl group has from 1 to 8 carbon atoms. Representative straight chain “—₁-C₈ alkyl” groups include, but are not limited to, -methyl, -ethyl, -n-propyl, -n-butyl, -n-pentyl, -n-hexyl, -n-heptyl and -n-octyl: while branched C₃-C₈ alkyls include, but are not limited to, -isopropyl, -sec-butyl, -isobutyl, -tert-butyl, -isopentyl, and -2-methylbutyl; unsaturated C₂-C₈ alkyls include, but are not limited to, -vinyl, -allyl, -1-butenyl, -2-butenyl, -isobu-tylenyl, -1 pentenyl, -2 pentenyl, -3-methyl-1-butenyl, -2 methyl -2-butenyl, -2,3 dimethyl -2-butenyl, -1-hexyl, 2-hexyl, -3-hexyl, -acetylenyl, -propynyl, -1 butynyl, -2 butynyl, -1 pentynyl, -2 pentynyl and -3 methyl 1 butynyl. Sometimes an alkyl group is unsubstituted. An alkyl group can be substituted with one or more groups. In other aspects, an alkyl group will be saturated.

As used herein, the term “optionally substituted alkyl” refers to unsubstituted alkyl or alkyl having designated substituents replacing one or more hydrogen atoms on one or more carbons of the hydrocarbon backbone. Such substituents can include, for example, alkyl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and acylamino), acylamino (including alkylcarbonylamino, arylcarbonylamino, carbamoyl and ureido), amidino, imino, sulphhydryl, alkylthio, arylthio, thiocarboxylate, sulphates, alkylsulphinyl, sulphonato, sulphamoyl, sulphonamido, nitro, trifluoromethyl, cyano, azido, heterocyclyl, alkylaryl, or an aromatic or heteroaromatic moiety.

Unless otherwise indicated, “alkylene”, by itself of as part of another term, refers to a substituted or saturated, branched or straight chain or cyclic hydrocarbon radical of the stated number of carbon atoms, typically 1-10 carbon atoms, and having two monovalent radical centers derived by the removal of two hydrogen atoms from the same or two different carbon atoms of a parent alkane. Typical alkylene radicals include, but are not limited to: methylene (—CH₂—), 1,2-ethylene (—CH₂CH₂—), 1,3-propylene (—CH₂CH₂CH₂—), 1,4-butylene (—CH₂CH₂CH₂CH₂—), and the like. In preferred aspects, an alkylene is a branched or straight chain hydrocarbon (i.e., it is not a cyclic hydrocarbon).

Unless otherwise indicated, “aryl”, by itself or as part of another term, means a substituted or monovalent carbocyclic aromatic hydrocarbon radical of the stated number of carbon atoms, typically 6-20 carbon atoms, derived by the removal of one hydrogen atom from a single carbon atom of a parent aromatic ring system. Some aryl groups are represented in the exemplary structures as “Ar”. Typical aryl groups include, but are not limited to, radicals derived from benzene, substituted benzene, naphthalene, anthracene, biphenyl, and the like. An exemplary aryl group is a phenyl group.

As used herein, the term “heterocycloalkyl” refers to a saturated or partially unsaturated 3-8 membered monocyclic or 6-10 membered bicyclic (fused, bridged, or spiro) ring system having one or more heteroatoms independently selected from the group consisting of nitrogen, oxygen and sulphur, unless specified otherwise. Examples of heterocycloalkyl groups include, but are not limited to, piperidinyl, piperazinyl, pyrrolidinyl, dioxanyl, tetrahydrofuranyl, isoindolinyl, indolinyl, imidazolidinyl, pyrazolidinyl, oxazolidinyl, isoxazolidinyl, triazolidinyl, oxiranyl, azetidinyl, oxetanyl, thietanyl, 1,2,3,6-tetrahydropyridinyl, tetrahydropyranyl, dihydropyranyl, pyranyl, morpholinyl, tetrahydrothiopyranyl, 1,4-diazepanyl, 1,4-oxazepanyl, 2-oxa-5-azabicyclo[2.2.1]heptanyl, 2,5-diazabicyclo[2.2.1]heptanyl, 2-oxa-6-azaspiro[3.3]heptanyl, 2,6-diazaspiro[3.3]heptanyl, 1,4-dioxa-8-azaspiro[4.5]decanyl, 1,4-dioxaspiro[4.5]decanyl, 1-oxaspiro[4.5]decanyl, 1-azaspiro[4.5]decanyl, 3′H-spiro[cyclohexane-1, 1′-isobenzofuran]-yl, 7′H-spiro[cyclohexane-1,5′-furo[3,4-b]pyridin]-yl, 3′H-spiro[cyclohexane-1, 1′-furo[3,4-c]pyridin]-yl, 3-azabicyclo[3.1.0]hexanyl, 3-azabicyclo[3.1.0]hexan-3-yl, 1,4,5,6-tetrahydropyrrolo[3,4-c]pyrazolyl, 3,4,5,6, 7,8-hexahydropyrido[4,3-d]pyrimidinyl, 4,5,6,7-tetrahydro-1H-pyrazolo[3,4-c]pyridinyl, 5,6, 7,8-tetrahydropyrido[4,3-d]pyrimidinyl, 2-azaspiro[3.3]heptanyl, 2-methyl-2-azaspiro[3.3]heptanyl, 2-azaspiro[3.5]nonanyl, 2-methyl-2-azaspiro[3.5]nonanyl, 2-azaspiro[4.5]decanyl, 2-methyl-2-azaspiro[4.5]decanyl, 2-oxa-azaspiro[3.4]octanyl, 2-oxa-azaspiro[3.4]octan-6-yl, and the like. In the case of multicyclic heterocycloalkyl, only one of the rings in the heterocycloalkyl needs to be non-aromatic.

As used herein, the term “heteroaryl” is intended to include a stable 5-, 6-, or 7-membered monocyclic or 7-, 8-, 9-, or 10-membered bicyclic aromatic heterocyclic ring which consists of carbon atoms and one or more heteroatoms independently selected from the group consisting of nitrogen, oxygen and sulphur. The nitrogen atom may be substituted or unsubstituted (i.e., N or NR wherein R is H or other substituents, as defined). The nitrogen and sulphur heteroatoms may optionally be oxidised (i.e., N→O and S(O)ₚ, where p=1 or 2). It is to be noted that total number of S and O atoms in the aromatic heterocycle is not more than 1. Examples of heteroaryl groups include pyrrole, furan, thiophene, thiazole, isothiazole, imidazole, triazole, tetrazole, pyrazole, oxazole, isoxazole, pyridine, pyrazine, pyridazine, pyrimidine, and the like.

Unless otherwise indicated, the term “heteroalkyl” by itself or in combination with another term, means, unless otherwise stated, a stable straight or branched chain hydrocarbon, or combinations thereof, fully saturated or containing from 1 to 3 degrees of unsaturation, consisting of the stated number of carbon atoms and from one to ten, preferably one to three, heteroatoms selected from the group consisting of O, N, Si and S, and wherein the nitrogen and sulfur atoms may optionally be oxidized and the nitrogen heteroatom may optionally be quaternized. The heteroatom (s) O, N and S may be placed at any interior position of the heteroalkyl group or at the position at which the alkyl group is attached to the remainder of the molecule. The heteroatom Si may be placed at any position of the heteroalkyl group, including the position at which the alkyl group is attached to the remainder of the molecule. Examples include —CH₂—CH₂—O—CH₃, —CH₂—CH₂—NH—CH₃, —CH₂—CH₂—N(CH₃)—CH₃, —CH₂—S—CH₂—CH₃, —CH₂—CH₂—S(O)—CH₃, —NH—CH₂—CH₂—NH—C(O)—CH₂—CH₃, —CH₂—CH₂—S(O)₂—CH₃, —CH═CH—O—CH₃, —Si(CH₃)₃, —CH₂—CH═N—O—CH₃, and —CH═CH—N(CH₃)—CH₃. Up to two heteroatoms may be consecutive, such as, for example, —CH₂—NH—OCH₃ and —CH₂—O—Si(CH₃)₃. Typically, a C₁ to C₄ heteroalkyl or heteroalkylene has 1 to 4 carbon atoms and 1 or 2 heteroatoms and a C₁ to C₃ heteroalkyl or heteroalkylene has 1 to 3 carbon atoms and 1 or 2 heteroatoms. In some aspects, a heteroalkyl or heteroalkylene is saturated.

Unless otherwise indicated, the term “heteroalkylene” by itself or in combination with another term means a divalent group derived from heteroalkyl (as discussed above), as exemplified by —CH₂—CH₂—S—CH₂—CH₂— and —CH₂—S—CH₂—CH₂—NH—CH₂—. For heteroalkylene groups, heteroatoms can also occupy either or both of the chain termini. Still further, for alkylene and heteroalkylene linking groups, no orientation of the linking group is implied.

“Protecting group” as used here means a moiety that prevents or reduces the ability of the atom or functional group to which it is linked from participating in unwanted reactions. Typical protecting groups for atoms or functional groups are given in Greene (1999), “PROTECTIVE GROUPS IN ORGANIC SYNTHESIS, 3^{RD} ED.”, Wiley Interscience. Protecting groups for heteroatoms such as oxygen, sulfur and nitrogen are used in some instances to minimize or avoid unwanted their reactions with electrophilic compounds. In other instances, the protecting group is used to reduce or eliminate the nucleophilicity and/or basicity of the unprotected heteroatom. Non-limiting examples of protected oxygen are given by —OR^{PR}, wherein R^{PR} is a protecting group for hydroxyl, wherein hydroxyl is typically protected as an ester (e.g. acetate, propionate or benzoate). Other protecting groups for hydroxyl avoid interfering with the nucleophilicity of organometahic reagents or other highly basic reagents, where hydroxyl is typically protected as an ether, including alkyl or heterocycloalkyl ethers, (e.g., methyl or tetrahydropyranyl ethers), alkoxymethyl ethers (e.g., methoxymethyl or ethoxymethyl ethers), optionally substituted aryl ethers, and silyl ethers (e.g., trimethylsilyl (TMS), triethylsilyl (TES), tert-butyldiphenylsilyl (TBDPS), tert-butyldimethylsilyl (TBS/TBDMS), triisopropylsilyl (TIPS) and [2-(trimethylsilyl)ethoxy]-methylsilyl (SEM)). Nitrogen protecting groups include those for primary or secondary amines as in —NHR^{PR} or —N(R^{PR})₂—, wherein least one of R^{PR} is a nitrogen atom protecting group or both R^{PR} together comprise a protecting group.

A protecting group is suitable when it is capable of preventing or avoiding unwanted side-reactions or premature loss of the protecting group under reaction conditions required to effect desired chemical transformation elsewhere in the molecule and during purification of the newly formed molecule when desired, and can be removed under conditions that do not adversely affect the structure or stereochemical integrity of that newly formed molecule. By way of example and not limitation, a suitable protecting group may include those previously described for protecting functional groups. A suitable protecting group is sometimes a protecting group used in peptide coupling reactions.

“Arylalkyl” or “heteroarylalkyl” as used herein means a substituent, moiety or group where an aryl moiety is bonded to an alkyl moiety, i.e., aryl-alkyl-, where alkyl and aryl groups are as described above, e.g., C₆H₅—CH₂— or C₆H₅—CH(CH₃)CH₂—. An arylalkyl or heteroary lalkyl is associated with a larger structure or moiety through a sp³ carbon of its alkyl moiety. A “metabolite” is a product produced through metabolism in the body of a specified compound, a derivative thereof, or a conjugate thereof, or salt thereof. Metabolites of a compound, a derivative thereof, or a conjugate thereof, may be identified using routine techniques known in the art and their activities determined using tests such as those described herein. Such products may result for example from the oxidation, hydroxylation, reduction, hydrolysis, amidation, deamidation, esterification, deesterification, enzymatic cleavage, and the like, of the administered compound. Accordingly, the invention includes metabolites of compounds, a derivative thereof, or a conjugate thereof, of the invention, including compounds, a derivative thereof, or a conjugate thereof, produced by a process comprising contacting a compound, a derivative thereof, or a conjugate thereof, of this invention with a mammal for a period of time sufficient to yield a metabolic product thereof.

As used herein, the term “pharmaceutically acceptable salt” refers to organic or inorganic salts of a compound of the present disclosure that have specified toxicity and/or biodistribution properties. Suitable salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, and/or pamoate (i.e., 1,1′-methylene-bis-(2-hydroxy-3-naphthoate)) salts. The pharmaceutically acceptable salt may balance charge on the parent compound by being present as a counterion. More than one counterion may be present. When multiple counterions are present, the compounds may be present as a mixed pharmaceutically acceptable salt.

Pharmaceutically acceptable salts and/or hydrates of the mitofusin activators may also be present in the compositions of the present disclosure. As used herein, the term “pharmaceutically acceptable solvate” refers to an association between one or more solvent molecules and a mitofusin activator of the present disclosure or a salt thereof, wherein the solvate has specified toxicity and/or biodistribution properties. Examples of solvents that may form pharmaceutically acceptable solvates include, but are not limited to, water, isopropanol, ethanol, methanol, DMSO, ethyl acetate, acetic acid, and/or ethanolamine. As used herein, the term “pharmaceutically acceptable hydrate” refers to a mitofusin activator of the present disclosure or a salt thereof that further includes a stoichiometric or non-stoichiometric amount of water bound by non-covalent intermolecular forces, wherein the hydrate has specified toxicity and/or biodistribution properties.

The mitofusin activators described herein may be formulated using one or more pharmaceutically acceptable excipients (carriers) known to persons having ordinary skill in the art. The term “pharmaceutically acceptable excipient,” as used herein, refers to substances or components that do not cause unacceptable losses of pharmacological activity or unacceptable adverse side effects when administered to a subject. Example “pharmaceutically acceptable excipients” include, but are not limited to, solvents, dispersion media, coatings, antibacterial agents, antifungal agents, isotonic, and absorption delaying agents, provided that any of these agents do not produce significant side effects or are incompatible with the mitofusin activator in the composition. Example excipients are described, for example, in Remington's Pharmaceutical Sciences (A.R. Gennaro, Ed.), 21st edition, ISBN: 0781746736 (2005) and United States Pharmacopeia (USP 29) and National Formulary (NF 24), United States Pharmacopeial Convention, Inc, Rockville, Maryland, 2005 (“USP/NF”), or a more recent edition, and the components listed in the continuously updated Inactive Ingredient Search online database of the FDA. Other useful components that are not described in the USP/NF may also be used. Such formulations may contain a therapeutically effective amount of one or more mitofusin activators, optionally as a salt, hydrate, and/or solvate, together with a suitable amount of excipient to provide a form for proper administration to a subject.

Compositions of the present disclosure may be stable to specified storage conditions. A “stable” composition refers to a composition having sufficient stability to allow storage at a convenient temperature, such as from about 0° C. to about 60° C. or about −20° C. to about 50° C., for a commercially reasonable period of time, such as at least about one day, at least about one week, at least about one month, at least about three months, at least about six months, at least about one year, or at least about two years.

Compositions of the present disclosure may be tailored to suit a desired mode of administration, which may include, but are not limited to, parenteral, pulmonary, oral, topical, transdermal, intradermal, intramuscular, intraperitoneal, intravenous, subcutaneous, intranasal, epidural, ophthalmic, pulmonary, epidural, buccal, and rectal. The compositions may also be administered in combination with one or more additional agents or together with other biologically active or biologically inert agents.

Controlled-release (or sustained-release) compositions may be formulated to extend the activity of the mitofusin activators and reduce dosing frequency. Controlled-release compositions may also be used to affect the time of onset of action or other characteristics, such as plasma levels of the mitofusin activator, and consequently affect the occurrence of side effects. Controlled-release compositions may be designed to initially release an amount of one or more mitofusin activators that produces the desired therapeutic effect, and gradually and continually release other amounts of the mitofusin activator to maintain the level of therapeutic effect over an extended period. In order to maintain a near-constant level of mitofusin activator in the body, the mitofusin activator may be released at a rate sufficient to replace the amount being metabolized or excreted from a subject. The controlled-release may be stimulated by various inducers (e.g., change in pH, change in temperature, enzymes, water, or other physiological conditions or molecules).

Agents or compositions described herein may also be used in combination with other therapeutic modalities, as described further below. Thus, in addition to the therapies described herein, one may also provide to the subject other therapies known to be efficacious for treatment of a disease, disorder, or condition being targeted by the mitofusin activator or a related disease, disorder, or condition.

Mitofusin activators of the present disclosure may stimulate mitochondrial fusion, increase mitochondrial fitness, and enhance mitochondrial subcellular transport. Accordingly, in another aspect of the present disclosure, any one or a combination of mitofusin activators of the present disclosure or a pharmaceutically acceptable salt thereof may be administered in a therapeutically effective amount to a subject having or suspected of having a mitochondria-associated disease, disorder or condition. The subject may be a human or other mammal having or suspected of having a mitochondria-associated disease, disorder or condition.

The mitochondria-associated disease, disorder or condition may be a pheripheral nervous system (PNS) or central nervous system (CNS) genetic or non-genetic disorder, physical damage, and/or chemical injury. In some aspects, in the method of treating a disease, disorder or condition for which a mitofusin activator is indicated, the PNS or CNS disorder may be selected from any one or a combination of: a chronic neurodegenerative condition wherein mitochondrial fusion, fitness, or trafficking are impaired: a disease or disorder associated with mitofusin-1 (MFN1) or mitofusin-2 (MFN2) dysfunction; a disease associated with mitochondrial fragmentation, dysfunction, or dysmotility; a degenerative neuromuscular condition such as Charcot-Marie-Tooth disease, amyotrophic lateral sclerosis (ALS), Huntington's disease, Alzheimer's disease, Parkinson's disease, hereditary motor and sensory neuropathy, autism, autosomal dominant optic atrophy (ADOA), muscular dystrophy, Lou Gehrig's disease, cancer, mitochondrial myopathy, diabetes mellitus and deafness (DAD), Leber's hereditary optic neuropathy (LHON), Leigh syndrome, subacute sclerosing encephalopathy, neuropathy, ataxia, retinitis pigmentosa, and ptosis (NARP), myoneurogenic gastrointestinal encephalopathy (MNGIE), myoclonic epilepsy with ragged red fibers (MERRF), mitochondrial myopathy, encephalomyopathy, lactic acidosis, stroke-like symptoms (MELAS), mtDNA depletion, mitochondrial neurogastrointestinal encephalomyopathy (MNGIE), dysautonomic mitochondrial myopathy, mitochondrial channelopathy, or pyruvate dehydrogenase complex deficiency (PDCD/PDH), diabetic neuropathy, chemotherapy-induced peripheral neuropathy, crush injury, SCI, traumatic brain injury (TBI), stroke, optic nerve injury, and/or related conditions that involve axonal disconnection.

Other mitochondria-associated diseases, disorders, or conditions that may be treated with the compositions disclosed herein, but are not limited to, Alzheimer's disease, ALS, Alexander disease, Alpers' disease, Alpers-Huttenlocher syndrome, alpha-methylacyl-CoA racemase deficiency, Andermann syndrome, Arts syndrome, ataxia neuropathy spectrum, ataxia (e.g., with oculomotor apraxia, autosomal dominant cerebellar ataxia, deafness, and narcolepsy), autosomal recessive spastic ataxia of Charlevoix-Saguenay, Batten disease, beta-propeller protein-associated neurodegeneration, cerebro-oculo-facio-skeletal syndrome (COFS), corticobasal degeneration, CLN1 disease, CLN10) disease, CLN2 disease, CLN3 disease, CLN4 disease, CLN6 disease, CLN7 disease, CLN8 disease, cognitive dysfunction, congenital insensitivity to pain with anhidrosis, dementia, familial encephalopathy with neuroserpin inclusion bodies, familial British dementia, familial Danish dementia, fatty acid hydroxylase-associated neurodegeneration, Friedreich's Ataxia. Gerstmann-Straussler-Scheinker Disease, GM2-gangliosidosis (e.g., AB variant), HMSN type 7 (e.g., with retinitis pigmentosa), Huntington's disease, infantile neuroaxonal dystrophy, infantile-onset ascending hereditary spastic paralysis, infantile-onset spinocerebellar ataxia, juvenile primary lateral sclerosis, Kennedy's disease, Kuru, Leigh's Disease, Marinesco-Sjögren syndrome, mild cognitive impairment (MCI), mitochondrial membrane protein-associated neurodegeneration, motor neuron disease, monomelic amyotrophy, motor neuron diseases (MND), multiple system atrophy, multiple system atrophy with orthostatic hypotension (Shy-Drager Syndrome), multiple sclerosis, multiple system atrophy, neurodegeneration in down's syndrome (NDS), neurodegeneration of aging, neurodegeneration with brain iron accumulation, neuromyelitis optica, pantothenate kinase-associated neurodegeneration, opsoclonus myoclonus, prion disease, progressive multifocal leukoencephalopathy, Parkinson's disease, Parkinson's disease-related disorders, polycystic lipomembranous osteodysplasia with sclerosing leukoencephalopathy, prion disease, progressive external ophthalmoplegia, riboflavin transporter deficiency neuronopathy, Sandhoff disease, spinal muscular atrophy (SMA), spinocerebellar ataxia (SCA), striatonigral degeneration, transmissible spongiform encephalopathies (prion diseases), and/or Wallerian-like degeneration.

Still other mitochondria-associated diseases, disorders, or conditions that may be treated with the compositions disclosed herein include abulia; agraphia; alcoholism; alexia; alien hand syndrome; Allan-Herndon-Dudley syndrome; alternating hemiplegia of childhood; Alzheimer's disease; amaurosis fugax; amnesia; ALS; aneurysm; angelman syndrome; anosognosia; aphasia; apraxia; arachnoiditis; Arnold-Chiari malformation; asomatognosia; Asperger syndrome; ataxia; attention deficit hyperactivity disorder; atr-16 syndrome; auditory processing disorder; autism spectrum; Behcets disease; bipolar disorder; Bell's palsy; brachial plexus injury; brain damage; brain injury; brain tumor; Brody myopathy; Canavan disease; capgras delusion; carpal tunnel syndrome; causalgia; central pain syndrome; central pontine myelinolysis; centronuclear myopathy; cephalic disorder; cerebral aneurysm; cerebral arteriosclerosis; cerebral atrophy; cerebral autosomal dominant arteriopathy with subcortical infarcts and leukoencephalopathy (CADASIL); cerebral dysgenesis-neuropathy-ichthyosis-keratoderma syndrome (CEDNIK syndrome); cerebral gigantism; cerebral palsy; cerebral vasculitis; cervical spinal stenosis; Charcot-Marie-Tooth disease; chiari malformation; chorea; chronic fatigue syndrome; chronic inflammatory demyelinating polyneuropathy (CIDP); chronic pain; Cockayne syndrome; Coffin-Lowry syndrome; coma; complex regional pain syndrome; compression neuropathy; congenital facial diplegia; corticobasal degeneration; cranial arteritis; craniosynostosis; Creutzfeldt-Jakob disease; cumulative trauma disorders; Cushing's syndrome; cyclothymic disorder; cyclic vomiting syndrome (CVS); cytomegalic inclusion body disease (CIBD); cytomegalovirus infection; Dandy-Walker syndrome; dawson disease; de Morsier's syndrome; Dejerine-Klumpke palsy; Dejerine-Sottas disease; delayed sleep phase syndrome; dementia; dermatomyositis; developmental coordination disorder; diabetic neuropathy; diffuse sclerosis; diplopia; disorders of consciousness; down syndrome; Dravet syndrome; duchenne muscular dystrophy; dysarthria; dysautonomia; dyscalculia; dysgraphia; dyskinesia; dyslexia; dystonia; empty sella syndrome; encephalitis: encephalocele; encephalotrigeminal angiomatosis; encopresis; enuresis; epilepsy; epilepsy-intellectual disability in females; erb's palsy; erythromelalgia; essential tremor; exploding head syndrome; Fabry's disease; Fahr's syndrome; fainting; familial spastic paralysis; febrile seizures; Fisher syndrome; Friedreich's ataxia; fibromyalgia; Foville's syndrome; fetal alcohol syndrome; fragile x syndrome; fragile x-associated tremor/ataxia syndrome (FXTAS); Gaucher's disease; generalized epilepsy with febrile seizures plus; Gerstmann's syndrome; giant cell arteritis; giant cell inclusion disease; globoid cell leukodystrophy; gray matter heterotopia; Guillain-Barré syndrome; generalized anxiety disorder; HTLV-1 associated myelopathy; Hallervorden-Spatz syndrome; head injury; headache; hemifacial spasm; hereditary spastic paraplegia; heredopathia atactica polyneuritiformis; herpes zoster oticus; herpes zoster; Hirayama syndrome; Hirschsprung's disease; Holmes-Adie syndrome; holoprosencephaly; Huntington's disease; hydranencephaly; hydrocephalus; hypercortisolism; hypoxia; immune-mediated encephalomyelitis; inclusion body myositis; incontinentia pigmenti; infantile refsum disease; infantile spasms; inflammatory myopathy; intracranial cyst; intracranial hypertension; isodicentric 15; Joubert syndrome; Karak syndrome; Kearns-Sayre syndrome; Kinsbourne syndrome; Kleine-Levin syndrome; Klippel Feil syndrome; Krabbe disease; Kufor-Rakeb syndrome; Lafora disease; Lambert-Eaton myasthenic syndrome; Landau-Kleffner syndrome; lateral medullary (Wallenberg) syndrome; learning disabilities; Leigh's disease; Lennox-Gastaut syndrome; Lesch-Nyhan syndrome; leukodystrophy; leukoencephalopathy with vanishing white matter; lewy body dementia; lissencephaly; locked-in syndrome; Lou Gehrig's disease (amyotrophic lateral sclerosis (ALS)); lumbar disc disease: lumbar spinal stenosis; lyme disease-neurological sequelae; Machado-Joseph disease (spinocerebellar ataxia type 3); macrencephaly; macropsia; mal de debarquement; megalencephalic leukoencephalopathy with subcortical cysts; megalencephaly; Melkersson-Rosenthal syndrome; menieres disease; meningitis; Menkes disease; metachromatic leukodystrophy; microcephaly; micropsia; migraine; Miller Fisher syndrome; mini-stroke (transient ischemic attack); misophonia; mitochondrial myopathy; mobius syndrome; monomelic amyotrophy; Morvan syndrome; motor neurone disease—see ALS; motor skills disorder; moyamoya disease; mucopolysaccharidoses; multi-infarct dementia; multifocal motor neuropathy; multiple sclerosis; multiple system atrophy; muscular dystrophy; myalgic encephalomyelitis; myasthenia gravis; myelinoclastic diffuse sclerosis; myoclonic encephalopathy of infants; myoclonus; myopathy; myotubular myopathy; myotonia congenita; narcolepsy; neuro-Behçet's disease; neurofibromatosis; neuroleptic malignant syndrome; neurological manifestations of aids; neurological sequelae of lupus; neuromyotonia; neuronal ceroid lipofuscinosis; neuronal migration disorders; neuropathy; neurosis; Niemann-Pick disease; non-24-hour sleep-wake disorder; nonverbal learning disorder; O'Sullivan-McLeod syndrome; occipital neuralgia; occult spinal dysraphism sequence; Ohtahara syndrome; olivopontocerebellar atrophy; opsoclonus myoclonus syndrome; optic neuritis; orthostatic hypotension; otosclerosis; overuse syndrome; palinopsia; paresthesia; Parkinson's disease; paramyotonia congenita; paraneoplastic diseases; paroxysmal attacks; Parry-Romberg syndrome; pediatric autoimmune neuropsychiatric disorders associated with streptococcoal infections (PANDAS); Pelizaeus-Merzbacher disease; periodic paralyses; peripheral neuropathy; pervasive developmental disorders; phantom limb/phantom pain; photic sneeze reflex; phytanic acid storage disease; Pick's disease; pinched nerve; pituitary tumors; pmg: polyneuropathy; polio; polymicrogyria; polymyositis; porencephaly; post-polio syndrome; postherpetic neuralgia (phn); postural hypotension; Prader-Willi syndrome; primary lateral sclerosis; prion diseases; progressive hemifacial atrophy; progressive multifocal leukoencephalopathy; progressive supranuclear palsy; prosopagnosia; pseudotumor cerebri; quadrantanopia; quadriplegia; rabies; radiculopathy; Ramsay Hunt syndrome type 1; Ramsay Hunt syndrome type 2; Ramsay Hunt syndrome type 3 —see Ramsay-Hunt syndrome; Rasmussen encephalitis; reflex neurovascular dystrophy; refsum disease; REM sleep behavior disorder; repetitive stress injury; restless legs syndrome; retrovirus-associated myelopathy; Rett syndrome; Reye's syndrome; rhythmic movement disorder; Romberg syndrome; Saint Vitus' dance; Sandhoff disease; Schilder's disease (two distinct conditions); schizencephaly; sensory processing disorder; septo-optic dysplasia; shaken baby syndrome; shingles; Shy-Drager syndrome; Sjögren's syndrome; sleep apnea; sleeping sickness; snatiation; Sotos syndrome; spasticity; spina bifida; spinal cord injury; spinal cord tumors; spinal muscular atrophy; spinal and bulbar muscular atrophy; spinocerebellar ataxia; split-brain; Steele-Richardson-Olszewski syndrome; stiff-person syndrome; stroke; Sturge-Weber syndrome; stuttering; subacute sclerosing panencephalitis; subcortical arteriosclerotic encephalopathy; superficial siderosis; Sydenham's chorea; syncope; synesthesia; syringomyelia; tarsal tunnel syndrome; tardive dyskinesia; tardive dysphrenia; Tarlov cyst; Tay-Sachs disease; temporal arteritis; temporal lobe epilepsy; tetanus; tethered spinal cord syndrome; Thomsen disease; thoracic outlet syndrome; tic douloureux; Todd's Paralysis; tourette syndrome; toxic encephalopathy; transient ischemic attack; transmissible spongiform encephalopathies; transverse myelitis; traumatic brain injury; tremor; trichotillomania; trigeminal neuralgia; tropical spastic paraparesis; trypanosomiasis; tuberous sclerosis; 22q13 deletion syndrome; Unverricht-Lundborg disease; vestibular schwannoma (acoustic neuroma); Von Hippel-Lindau disease (VHL); viliuisk encephalomyelitis (VE); Wallenberg's syndrome; west syndrome; whiplash; Williams syndrome; Wilson's disease; y-linked hearing impairment; and/or Zellweger syndrome.

Each of the states, diseases, disorders, and conditions, described herein, as well as others, can benefit from compositions and methods described herein. Generally, treating a state, disease, disorder, or condition includes preventing or delaying the appearance of clinical symptoms in a mammal that may be afflicted with or predisposed to the state, disease, disorder, or condition but does not yet experience or display clinical or subclinical symptoms thereof. Treating can also include inhibiting the state, disease, disorder, or condition (e.g., arresting or reducing the development of the disease or at least one clinical or subclinical symptom thereof). Furthermore, treating can include relieving the disease (e.g., causing regression of the state, disease, disorder, or condition or at least one of its clinical or subclinical symptoms). A benefit to a subject to be treated can be either statistically significant or at least perceptible to the subject or to a physician.

A mitochondria-associated disease, disorder, or condition may be a disease primarily caused by or secondarily associated with mitochondrial dysfunction, fragmentation, or loss-of-fusion, or associated with dysfunction in MFN1 or MFN2 catalytic activity or conformational unfolding. Mitochondrial dysfunction may be caused by genetic mutations of mitofusins or other (nuclear or mitochondrial encoded) genes, or may be caused by physical, chemical, or environmental injury to the CNS or PNS.

In a particular example, cancer chemotherapy-induced sensory and motor neuropathies may be prevented or treated with the compositions of the present disclosure. Chemotherapy-induced peripheral neuropathy is one of the most common complications of cancer chemotherapy, affecting 20% of all patients and almost 100% of patients receiving high doses of chemotherapeutic agents. Dose-dependent neurotoxicity of motor and sensory neurons can lead to chronic pain, hypersensitivity to hot, cold, and mechanical stimuli, and/or impaired neuromuscular control. The most common chemotherapeutic agents linked to CIPN are platinum, vinca alkaloids, taxanes, epothilones, and the targeted proteasome inhibitor, bortezomib.

CIPN most commonly affects peripheral sensory neurons whose cell bodies are located in dorsal root ganglia lacking the blood-brain barrier that protects other components of the central and peripheral nervous system. Unprotected dorsal root ganglion neurons are more sensitive to neuronal hyperexcitability and innate immune system activation evoked by circulating cytotoxic chemotherapeutic agents. CIPN affects quality of life, and is potentially disabling, because it provokes chronic neuropathic pain that, like other causes of neuralgia (e.g., post herpetic neuralgia, diabetic mononeuropathy), is refractory to analgesic therapy. Motor nerve involvement commonly manifests as loss of fine motor function with deterioration in hand writing, difficulty in buttoning clothes or sewing, and sometimes upper and lower extremity weakness or loss of endurance. CIPN typically manifests within weeks of chemotherapy and in many cases improves after chemotherapy treatment ends, although residual pain, sensory, or motor defects are observed in one-third to one-half of affected patients. Unfortunately, CIPN-limited chemotherapy dosing can lead to delays, reduction, or interruption of cancer treatment, thus shortening survival.

Mitochondrial dysfunction and oxidative stress are implicated in CIPN because of observed ultrastructural morphological abnormalities, impaired mitochondria DNA transcription and replication, induction of mitochondrial apoptosis pathways, and reduction of experimental CIPN signs by anticipatory mitochondrial protection. Mitofusin activators may enhance overall mitochondrial function in damaged neurons, increase mitochondrial transport to areas of neuronal damage, and accelerate in vitro neuron repair/regeneration after chemotherapy-induced damage. For this reason, it is believed that mitofusin activators may reduce neuronal injury conferred by chemotherapeutic agents in CIPN and accelerate regeneration/repair of nerves damaged by chemotherapeutic anticancer agents. As such, the present disclosure provides for compositions and methods to treat cancer chemotherapy induced nerve injury and neuropathy.

In another example, injury in the CNS or PNS (e.g., trauma to the CNS or PNS, crush injury, SCI, TBI, stroke, optic nerve injury, or related conditions that involve axonal disconnection) may be treated with the compositions of the present disclosure. The CNS includes the brain and the spinal cord and the PNS is composed of cranial, spinal, and autonomic nerves that connect to the CNS.

Damage to the nervous system caused by mechanical, thermal, chemical, or ischemic factors may impair various nervous system functions such as memory, cognition, language, and voluntary movement. Most often, this is through accidental crush or transection of nerve tracts, or as an unintended consequence of medical interventions, that interrupt normal communications between nerve cell bodies and their targets. Other types of injuries may include disruption of the interrelations between neurons and their supporting cells or the destruction of the blood-brain barrier.

Mitofusin activators may rapidly reverse mitochondrial dysmotility in neurons from mice or patients with various genetic or chemotherapeutic neurodegenerative diseases, in axons injured by chemotherapeutic agents, and in axons severed by physical injury. For this reason, mitofusin activators may enhance regeneration/repair of physically damaged nerves, as in vehicular and sports injuries, penetration trauma from military or criminal actions, and iatrogenic injury during invasive medical procedures. As such, the present disclosure provides for compositions and methods to treat physical nerve injury.

Mitochondrial motility is also implicated in neuropathy and traumatic crush or severance nerve injuries. After nerve laceration or crush injury, nerves will either regenerate and restore neuromuscular function or fail to regenerate such that neuromuscular function in permanently impaired. Mitofusin activators may increase mitochondrial trafficking, thereby enabling a nerve to regenerate after traumatic injuries.

The amount of a mitofusin activator and excipient to produce a composition in a given dosage form may vary depending upon the subject being treated, the condition being treated and the particular mode of administration. It will be appreciated that the unit content of mitofusin activator contained in an individual dose of a given dosage form need not in itself constitute a therapeutically effective amount, as the necessary therapeutically effective amount could be reached by administration of a number of individual doses, or the therapeutic effect may be cumulative over time.

Dosing of the mitofusin activators of the present disclosure may occur as a single event or over a time course of treatment. For example, a mitofusin activator may be administered daily, weekly, bi-weekly, or monthly. For treatment of acute conditions, the time course of treatment may be at least several days, with dosing taking place at least once a day or continuously. Certain conditions could extend treatment from several days to several weeks. For example, treatment could extend over one week, two weeks, or three weeks. For chronic conditions, treatment could extend from several weeks to several months or even years.

Toxicity and therapeutic efficacy of the compositions described herein may be determined by standard pharmaceutical procedures in cell cultures or experimental animals for determining the LD₅₀ (the dose lethal to 50% of the population) and the ED₅₀, (the dose therapeutically effective in 50% of the population). The dose ratio between toxic and therapeutic effects is the therapeutic index that may be expressed as the ratio LD₅₀/ED₅₀, where larger therapeutic indices are generally understood in the art to be optimal.

Unless indicated to the contrary, the numerical parameters set forth in the following specification and attached claims are approximations that may vary depending upon the desired properties sought to be obtained by the embodiments of the present invention. At the very least, and not as an attempt to limit the application of the doctrine of equivalents to the scope of the claim, each numerical parameter should at least be construed in light of the number of reported significant digits and by applying ordinary rounding techniques.

One or more illustrative embodiments incorporating various features are presented herein. Not all features of a physical implementation are described or shown in this application for the sake of clarity. It is understood that in the development of a physical embodiment incorporating the embodiments of the present invention, numerous implementation-specific decisions must be made to achieve the developer's goals, such as compliance with system-related, business-related, government-related and other constraints, which vary by implementation and from time to time. While a developer's efforts might be time-consuming, such efforts would be, nevertheless, a routine undertaking for those of ordinary skill in the art and having benefit of this disclosure.

While various systems, tools and methods are described herein in terms of “comprising” various components or steps, the systems, tools and methods can also “consist essentially of” or “consist of” the various components and steps.

As used herein, the phrase “at least one of” preceding a series of items, with the terms “and” or “or” to separate any of the items, modifies the list as a whole, rather than each member of the list (i.e., each item). The phrase “at least one of” allows a meaning that includes at least one of any one of the items, and/or at least one of any combination of the items, and/or at least one of each of the items. By way of example, the phrases “at least one of A, B, and C” or “at least one of A, B, or C” each refer to only A, only B, or only C; any combination of A, B, and C; and/or at least one of each of A, B, and C.

Therefore, the disclosed systems, tools and methods are well adapted to attain the ends and advantages mentioned as well as those that are inherent therein. The particular embodiments disclosed above are illustrative only, as the teachings of the present disclosure may be modified and practiced in different but equivalent manners apparent to those skilled in the art having the benefit of the teachings herein. Furthermore, no limitations are intended to the details of construction or design herein shown, other than as described in the claims below. It is therefore evident that the particular illustrative embodiments disclosed above may be altered, combined, or modified and all such variations are considered within the scope of the present disclosure. The systems, tools and methods illustratively disclosed herein may suitably be practiced in the absence of any element that is not specifically disclosed herein and/or any optional element disclosed herein. While systems, tools and methods are described in terms of “comprising,” “containing,” or “including” various components or steps, the systems, tools and methods can also “consist essentially of” or “consist of” the various components and steps. All numbers and ranges disclosed above may vary by some amount. Whenever a numerical range with a lower limit and an upper limit is disclosed, any number and any included range falling within the range is specifically disclosed. In particular, every range of values (of the form, “from about a to about b,” or, equivalently, “from approximately a to b,” or, equivalently, “from approximately a-b”) disclosed herein is to be understood to set forth every number and range encompassed within the broader range of values. Also, the terms in the claims have their plain, ordinary meaning unless otherwise explicitly and clearly defined by the patentee. Moreover, the indefinite articles “a” or “an,” as used in the claims, are defined herein to mean one or more than one of the elements that it introduces. If there is any conflict in the usages of a word or term in this specification and one or more patent or other documents that may be incorporated herein by reference, the definitions that are consistent with this specification should be adopted.

All publications and patent documents cited herein are incorporated herein by reference as if each such publication or document was specifically and individually indicated to be incorporated herein by reference. Citation of publications and patent documents is not intended as an admission that any is pertinent prior art, nor does it constitute any admission as to the contents or date of the same. The invention having now been described by way of written description, those of skill in the art will recognize that the invention can be practiced in a variety of embodiments and that the foregoing description and examples below are for purposes of illustration and not limitation of the claims that follow.

### EXAMPLES

### Exemplary Materials and Methods

### Cell Lines

Wild-type MEFs were prepared from E10.5 c57/b16 mouse embryos. SV-40 T antigen-immortalized MFN1 null (CRL-2992), MFN2 null (CRL-2993) and MFN1/MFN2 double null MEFs (CRL-2994) were purchased from ATCC. MEFs were subcultured in DMEM (4.5 g/L glucose) plus 10% fetal bovine serum, 1×nonessential amino acids, 2 mM L-glutamine, 100 units/mL penicillin and 100 μg/mL streptomycin.

### Confocal Live Cell Studies of Mitochondria

Live cell imaging was performed on an Olympus Diaphot 200 fluorescence microscope equipped with a 60×water immersion objective. All live cells were grown on coated glass-bottom 12-well plates and studied in modified Krebs-Henseleit buffer (138 mM NaCl, 3.7 mM KCl, 1.2 mM KH₂PO₄, 15 mM, 20 mM HEPES and 1 mM CaCl₂)) at room temperature.

Cells were excited with 408 nm (Hoechst), 561 nm (MitoTracker Green and Calcein AM, GFP), or 637 nm (TMRE, MitoTracker Orange, Ethidium homodimer-1, and AF594-Dextran) laser diodes. For mitochondrial elongation studies, mitochondrial aspect ratio (long axis/short axis) was calculated using automated edge detection and Image J software. Mitochondrial depolarization was calculated as percent of green mitochondria visualized on MitoTracker Green and TMRE merged images, expressed as green/(green+yellow mitochondria)×100.

Mouse lines

SOD1-Gly93Ala (G93A) transgenic mice (B6SJL-Tg(SOD1*G93A)1Gur/J) and C57BL/6J mice were purchased from The Jackson Laboratory (Bar Harbor, Maine, USA; Stock #: 002726, Stock, 000664).

Cultured cells

Directly reprogrammed human motor neurons were generated from human dermal fibroblasts as described (Abernathy DG, Kim WK, McCoy MJ, Lake AM, Ouwenga R, Lee SW, et al. MicroRNAs Induce a Permissive Chromatin Environment that Enables Neuronal Subtype-Specific Reprogramming of Adult Human Fibroblasts. Cell Stem Cell. 2017:21(3), 332-348.e9, Franco A, Dang X, Walton EK, Ho JN, Zablocka B, Ly C, et al. Burst mitofusin activation reverses neuromuscular dysfunction in murine CMT2A. Elife. 2020:9:e61119). Adult mouse dorsal root ganglion (DRG) neurons were prepared from 8-12 week old C57BL/6J or SOD1G93A transgenic mice as described (Franco A, Dang X, Walton EK, Ho JN, Zablocka B, Ly C, et al. Burst mitofusin activation reverses neuromuscular dysfunction in murine CMT2A. Elife. 2020;9:e61119).

PCR genotyping of mutations in ALS and FTD patient fibroblasts

DNA was extracted from 5×10₆ primary human fibroblasts using the DNeasy blood & tissue kit (Qiagen, Cat#, 69506) according to manufacturer's protocol. PCR of SOD1, TDP43 and FUS gene fragments of interest was performed (initial denaturation at 95 degrees C. for 5 mins, followed by 30 cycles of denaturation: 95 degrees C., 30 sec, annealing: 55 degrees C. 30 sec, extension: 72 degrees C., 30 sec, final extension at 68 degrees C. for 5 min, then hold at 4 degrees C.) using Taq Plus Master Mix 2X (Cat#: BETAQR-L, Bulls eye), 50 ng of genomic DNA template, and the following primers:

ALS:
SOD1 L38V-fw
5′-CTTCACTGTGAGGGGTAAAGG-3′
SOD1 L38V-rv
5′-CTAGGGTGAACAAGTATGGG-3′
SOD1 I113T-fw
5′-TGTTTAGTGGCATCAGCCCT-3′
SOD1 I113T-rv
5′-ACCGCGACTAACAATCAAAGTG-3′
SOD1 L145F-fw
5′-GGTAGTGATTACTTGACAGCCCAA-3′
SOD1 L145F-rv
5′-GTTAAGGGGCCTCAGACTACAT-3′
TDP43 A382T-fw
5′-AACATGCAGAGGGAGCCAAA-3′
TDP43 A382T-rv
5′-ACCCTGCATTGGATGCTGAT-3′
FUS R521G-fw
5′-TACTCGCTGGGTTAGGTAGGA-3′
FUS R521G-rv
5′-ACGAGGGTAACACTGGGTACA-3′
Frontotemporal dementia:
PGRN M1L and A9D-fw
5′-GGGGCTAGGGTACTGAGTGA-3′
PGRN M1L and A9D-rv
5′-TGGCCAATCCAAGATGACCC-3′
MAPT R406W-fw
5′-CTTTCTCTGGCACTTCATCTC-3′
MAPT R406W-rv
5′-CCTCTCCACAATTATTGACCG-3′.

PCR products were purified using PureLink Quick Gel Extraction Kit (Invitrogen, Cat#: K21000-12) and sent to GENEWIZ for Sanger sequencing.

### Preparative HPLC

Purification was performed using HPLC (H₂O-MeOH, Agilent 1260 Infinity systems equipped with DAD and mass-detectors. Waters SunFire C18 OBD Prep Column, 100 Å, 5 μm, 19 mm×100 mm with SunFire C18 Prep Guard Cartridge, 100 Å, 10 μm, 19 mm×10 mm) was used for separation. The material was dissolved in 0.7 mL DMSO. Flow rate, 30 mL/minute. Purity of the obtained fractions was checked via analytical LCMS. Spectra were recorded for each fraction as it was obtained straight after chromatography in the solution form. The solvent was evaporated in the flow of N₂ at 80° C. On the basis of post-chromatography LCMS analysis, fractions were combined united. Solid fractions were dissolved in 0.5 mL MeOH and transferred into pre-weighted marked vials. Obtained solutions were again evaporated in the flow of N₂ at 80° C. After drying, products were characterized by LCMS, ¹H NMR, and ¹³C NMR.

### HPLC/HRMS (ESI)

LC/MS analysis was carried out using Agilent 1100 Series LC/MSD system with DAD\ELSD and Agilent LC\MSD VL (G1956A), SL (G1956B) mass-spectrometer or Agilent 1200 Series LC/MSD system with DAD\ELSD and Agilent LC\MSD SL (G6130A), SL (G6140A) mass-spectrometer. All the LC/MS data were obtained using positive/negative mode switching. The compounds were separated using a Zorbax SB-C18 1.8 μm 4.6×15 mm Rapid Resolution cartridge (PN 821975-932) under a mobile phase (A—ACN, 0.1% formic acid; B—water (0.1% formic acid)). Flow rate: 3 mL/minute; Gradient 0 minutes—100% B; 0.01 minute—100% B; 1.5 minutes—0% B; 1.8 minutes—0% B; 1.81 minutes—100% B; Injection volume 1 μL; Ionization mode atmospheric pressure chemical ionization (APCI), Scan range m/z 80-1000.

### Statistical Methods

Time-course and dose-response data are calculated for each study using GraphPad Prism. All data are reported as mean±SEM. Statistical comparisons (two-sided) used one-way ANOVA and Tukey's tests for multiple groups or Student's t-test for paired comparisons. p<0.05 was considered significant. In vitro pharmacokinetic analyses of mitofusin activators was performed at WuXi Apptec Co. Ltd.

Binding to human and CD-1 mouse plasma proteins was measured using equilibrium dialysis. Pooled individual frozen EDTA anticoagulated plasma mouse and human samples were used as test matrix. Warfarin was used as a positive control. The test compounds were spiked into blank matrix at the final concentration of 2 μM. A 150-μL aliquot of matrix sample was added to one side of the chamber in a 96-well equilibrium dialyzer plate (HTD dialysis) and an equal volume of dialysis buffer was added to the other side of the chamber. An aliquot of matrix sample was harvested before the incubation and used as T₀ samples for recovery calculation. The incubations were performed in triplicate. The dialyzer plate was placed in a humidified incubator and rotated slowly for four hours at 37° C. After incubation, the samples were taken from the matrix side as well as the buffer side. The plasma sample was matched with equal volume of blank buffer; and buffer samples were matched with equal volume of blank plasma. The matrix-matched samples were quenched with stop solution containing internal standard. All samples were analyzed by LC-MS/MS. All test compound concentrations in matrix and buffer samples are expressed as peak area ratios (PAR) of analyte/internal standard.

In vitro stability was measured in human and mouse liver microsomes. An intermediate solution (100 μM of small molecule) was initially prepared in methanol and subsequently used to prepare the working solution. This was achieved by a 10-fold dilution step of the intermediate solution in 100 mM potassium phosphate buffer. Ten microliters of a compound working solution or control working solution was added to all wells of a 96-well plate for the time points (minutes): T₀, T₅, T₁₀, T₂₀, T₃₀, T₆₀, NCF60, except the matrix blank. The microsome solution (680 μL/well) (#452117, Corning; Woburn, Mass., USA; #R1000, Xenotech; Kansas City, Kans., USA and #M1000, Xenotech; Kansas City, Kans., USA) was dispersed to 96-well plate as reservoir according to the plate map. Then, 80 μL/well was added to every plate by ADDA (Apricot Design Dual Arm, Apricot Designs, Inc., Covina, Calif., USA), and the mixture of microsome solution and compound were allowed to incubate at 37° C. for about 10 minutes. Next, 10 μL of 100 mM potassium phosphate buffer/well was added to NCF60 and incubated at 37° C. (timer 1 H was started). After pre-warming. 90 μL/well of NADPH (#00616, Sigma, Aldrich, St. Louis, Mo., USA) regenerating system was dispensed to 96-well plate as reservoir according to the plate map. Then 10 μL/well was added to every plate by ADDA to start reaction. To terminate the reaction, 300 μL/well of stop solution (cold in 4° C., including 100 ng/ml tolbutamide and 100 ng/ml labetalol as internal standards) was used, and sampling plates were agitated for approximately 10 minutes. The samples were next centrifuged at 4000 rpm for 20 minutes at 4° C. Supernatants were analyzed by LC-MS/MS.

### Parallel Artificial Membrane Permeability Assay (PAMPA)

A 10 μM solution of a small molecule in 5% DMSO (150 μL) was added to each well of the donor plate, whose PVDF membrane was pre-coated with 5 μL of 1% brain polar lipid extract (porcine)/dodecane mixture. Then, 300 μL of PBS was added to each well of the PTFE acceptor plate. The donor plate and acceptor plate were combined together and incubated for 4 hours at room temperature with shaking at 300 rpm. To prepare the T₀ sample, 20 μL of a donor solution was transferred to new well, followed by the addition of 250 μL PBS (DF: 13.5) and 130 μL of ACN (containing internal standard) as the T₀ sample. To prepare the acceptor sample, the plate was removed from incubator and 270 μL of the solution was transferred from each acceptor well and mixed with 130 μL ACN (containing internal standard) as an acceptor sample. To prepare the donor sample, 20 μL of the solution was transferred from each donor well and mixed with 250 μL PBS (DF, 13.5), 130 μL ACN (containing internal standard) as a donor sample. The acceptor samples and donor samples were analyzed by LC-MS/MS.

### Other Methods

HPLC analyses were conducted with a Kinetex C18 column (4.6×50 mm, 5 μm; Mobile phase A: 0.0375% TFA in water (v/v), B: 0.01875% TFA in Acetonitrile (v/v)) run at 50° C. with absorbance at 200 nm.

LC-MS/MS (ESI) was performed using 2 systems: 1) SHIMADZU LC-MS-2020 with LabSolution V5.72 analysis software and a CHROMALITH@FLASH RP-18E 25*2.0 mm column run at 50° C. with a PDA (220 and 254 nm) detector, acquired data in scan MS Mode (positive mode) with m/z=100-1000 scan range, drying gas (N₂) flow: 15 L/min, DL voltage, 120V and Quarry DC voltage: 20V, or 2) Agilent 1200/G6110A instrument with AgilentChemStation Rev. B. 04.03 software and an XBRIDGE C18 2.1*50 mm column run at 40° C. with DAD (220 nm)/ELSD detector, acquired data in scan MS Mode (positive mode) with m/z=100-1000 scan range, drying gas (N₂) flow: 10 L/min, 350° C., nebulizer pressure: 35 psi, capillary voltage, 2500V. NMR spectrometry was carried out on Brucker AVANCE NEO 400 MHz with a 5 mm PABBO BB/19F-1H/D Z-GRD probe.

Dose-response of mitofusin agonist fusogenicity was performed in Mfn1- or Mfn2-deficient MEFs (Mfn1-KO or Mfn2-KO MEFs) cultured at 37° C. and 5% CO₂-95% air. Cells were seeded on day 1 in 6 well plates at a density of 2×10⁴ cells per well and compounds added at 9 concentrations (0.5 nM-10 μM dissolved in DMSO) overnight. Mitochondria were then stained with MitoTracker Orange (200 nM; M7510; Invitrogen, Carlsbad, CA, USA). Nuclei were stained with Hoescht (10 μg/ml; Invitrogen, Thermo Fisher Scientific Cat: #H3570). Images were acquired at room temperature on a Nikon Ti Confocal microscope using a 60 X 1.3 NA oil-immersion objective in Krebs-Henseleit buffer (138 NaCl, 3.7 nM KCl, 1.2 nM KH₂PO₄, 15 nM Glucose, 20 nM HEPES pH: 7.2-7.5, and 1 mM CaCl₂). Laser excitation was 549 nm with emission at 590 nm for MitoTracker Orange and excitation at 306 nm with emission at 405 nm for Hoescht. Images were analyzed using ImageJ and fusogenicity quantified as mitochondrial aspect ratio (length/width), and were indexed to the maximal response elicited by Compound 6, a known mitofusin activator. Response curves were interpolated using the sigmoidal model using Prism 8 software. EC₅₀ values are reported as mean with 95% confidence limits for at least 3 independent experiments.

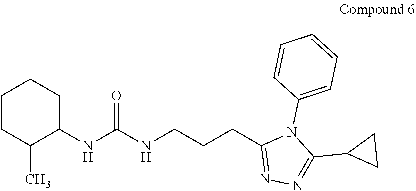 [SMILES: CC1CCCCC1NC(=O)NCCCc1nnc(C2CC2)n1-c1ccccc1]

1-(3-(5-cyclopropyl-4-phenyl-4H-1,2,4-triazol-3-yl)propyl)-3-(2-methylcyclohexyl)urea

Functional evaluation of mitofusin activation on mitochondrial depolarization was studied as follows. Cultured Mfn2-KO or Mfn1-KO MEFs were treated with DMSO, or Compounds 2A, 2B or 6 (1 μM) for 24 hours, then were stained with Tetramethylrhodamine ethyl ester (TMRE, 200 nM, Invitrogen Thermo Fisher Scientific Cat:#T-669), MitoTracker Green (200 nM; Invitrogen, Thermo Fisher Scientific Cat:# M7514) and Hoechst (10 ug/ml; Invitro-gen, Thermo Fisher Scientific Cat:#H3570) for 30 min at 37° C. in 5% CO2-95% air, washed twice in PBS. Images were acquired at room temperature on a Nikon Ti Confocal microscope using either 60 X 1.3 NA oil-immersion objective, in Krebs-Henseleit buffer (138 NaCl, 3.7 nM KCl, 1.2 nM KH₂PO₄, 15 nM glucose, 20 nM HEPES, pH: 7.2-7.5, and 1 mM CaCl₂): laser excitation was 488 nm with emission at 510 nm for MitoTracker Green, 549 nm with emission at 590 nm for TMRE, and 306 nm with emission 405 nm for Hoecsht. Mitochondrial depolarization was reported as % number of green mitochondria/number of yellow+green mitochondria using Image J.

In vitro pharmacokinetic analyses were performed in duplicate using standard methods by WuXi AppTec Co. Ltd. (Shanghai, China). Plasma protein binding was measured by equilibrium dialysis; % bound=(1-[free compound in dialysate]/[total compound in retentate])×100. Plasma stability of 2 μM compounds in clarified freeze-thawed plasma was assessed by LC-MS/MS of supernatants after protein precipitation; 120 min data are reported for studies including 0, 10, 30, 60, and 120 min. Liver microsome stability of 1 μM compounds in liver microsomes (0.5 mg/ml) after 0, 5, 10, 20, 30, 60 min. incubation was assessed by LC/MS/MS of reaction extracts. Passive artificial blood brain barrier membrane permeability assay (PAMPA-BBB) were performed using 150 μL of 10 μM compounds (5% DMSO) added to PVDF membranes pre-coated with 5 μL of 1% brain polar lipid extract (Porcine)/dodecane mixture and incubated for 4 h at room temperature with shaking at 300 rpm. Donor and acceptor samples were analyzed by LC-MS/MS.

In vivo pharmacokinetic analyses were performed in triplicate using standard methods by WuXi AppTec Co. Ltd. (Shanghai, China). Compounds (5 mg/mL) were dissolved in 10% DMSO/90% (30% cyclodextrin) and administered by oral administration (50 mg/kg) to 7-9 week male CD-1 mice (SLAC Laboratory Animal Co. Ltd., Shanghai, China or SIPPR/BK Laboratory Animal Co. Ltd., Shanghai, China). Plasma, brain, spinal cord and sciatic nerve concentration versus time data were analyzed by non-compartmental approaches using the Phoenix WinNonlin 6.3 software program, data are presented as mean from 3 mice for each condition.

In vivo and in vitro pharmacokinetic analyses involving mouse and human tissues were approved by Institutional Committee Animal Care and Use Committee, Shanghai Site (IACUC-SH); (WuXi Corporate Committee for Animal Research Ethics (WX-CCARE)) and done by WuXi AppTec Co. Ltd. (Shanghai, China). 2 mg/mL compounds were dispersed in 10% DMSO/90% (30% cyclodextrin) solution and administered to 7-9 week male CD-1 mice from SIPPR/BK Laboratory Animal Co. Ltd., Shanghai, China (15 mice per compound) by tail vein (10 mg/kg) or subcutaneously via osmotic mini-pump (60 mg/kg/day×three days).

### In Vivo Studies in ALS Mice (SOD1G93A)

Experimental design—60 day male and female old SODI G93A ALS mice were randomized to treatment with compound 2 (60 mg/kg PO twice daily) or the same vehicle (10% Me₂SO/90% (30% 2-hydroxypropyl)-β-cyclodextrin [HP-b-CD, Sigma, Cat:#332607]) (Compound 2A study). Drugs and vehicle were sterilefiltered (0.22 μm PVDF, #SLGV033RS, Millipore, Cork, Ireland) and syringes prepared and assigned to mice by LZ according to a randomization table. Drugs were administered to mice by XD who was blind to mouse genotype and treatment group. Behavioral and neurophysiological testing were performed before and every 10 days after initiation of therapy:

RotaRod testing was performed using a RotaRod from Ugo Basile (Gemonio, Italy; #47650). After initial training at a constant speed of 5 r.p.m. ,studies were performed with acceleration from 5 to 40 RPM over 120 seconds, maintaining 40 RPM thereafter. Mice were tested 5 times and the average latency time (when the mouse fell from the device) reported.

Inverted grip testing placed mice in the center of a tight woven mesh in an oval metal frame, which was inverted over 2 sec and maintained 40-50 cm above the bottom of cage until the mice fell (latency time). Studies were repeated three times and the average latency time reported.

A combined neuromuscular dysfunction score used the system described by Guyenet et al:

Ledge test: Score 0) (normal)=effectively use hind legs while walking along the ledge of the cage; Score 1=loses footing some times while walking along the ledge, but appears coordinated; Score 2=does not effectively use hind legs; Score 3=refuses to move along the ledge or falls off while walking; Hindlimb clasping, Score 0 (normal)=hindlimbs completely splayed outward while being lifted by its tail; Score 1=one hindlimb partially collapsed toward the abdomen; Score 2=both hindlimbs partially collapsed toward the abdomen; Score 3=hindlimbs entirely touching the abdomen; Gait: Score 0=normal gait; Score 1=tremor or limp; Score 2=feet point away from the body while walking; Score 3=difficulty moving forward; Kyphosis: Score 0 (normal)=able to straighten spine while walking, no kyphosis; Score 1=mild kyphosis but able to straighten spine; Score 2=unable to straighten spine with mild kyphosis; Score 3=severe kyphosis while walking and sitting. The results of each test were added together to obtain the combined neuromuscular dysfunction score.

Neuroelectrophysiologic recordings of tibialis gastrocnemius compound muscle action potentials (CMAP). Mice were anesthetized with isofluorane, shaved, and a needle electrode inserted to stimulate the proximal sciatic nerves (3.9 mV pulses; 0.002 ms duration). Ring electrodes were positioned at the mid forelimb to record CMAP with a Viasys Healthcare Nicolet Biomedical instrument (Middleton, WI, USA Cat:# OL060954) using Viking Quest version 11.2 software. Optimal stimulating electrode position was defined as that giving the greatest CMAP amplitude; 3-4 independent events were recorded and averaged.

Survival studies. Mice were observed until the level of neuromuscular dysfunction achieved the predetermined endpoint of being unable to right within 30 seconds of being placed on their backs.

Non-survival endpoint studies were terminated after final testing at the pre-determined age of 140 days. In the 140 day studies sciatic and mid tibial nerves, gastrocnemius muscles, and lumbar spinal cord samples were harvested and either frozen in optimal cutting temperature (OCT, Tissue-TEK Cat: 4583) or fixed in 4% PFA/PBS for 2 hours, transferred to 30% sucrose/PBS overnight at 4 degrees C., and embedded in paraffin. Nerve sections were stained with toluidine blue or immunolabelled with 4-HNE (1:200 in 10% goat serum, r.t., 0.5 hours, Abcam Cat#: ab46545) and b-tubulin III (1:200 in 10% goat serum, r.t., 0.5 hours, Biolegend Cat#: 801201). Gastrocnemius muscle sections were labelled with fluorescein-conjugated wheat germ agglutinin (WGA, Cat#: W834, Invitrogen) to label myocyte membranes and 4-HNE to label ROS for 30 mins at room temperature.

Neuromuscular junctions (NMJs) staining used 10 μm thick frozen sections of gastrocnemius

muscle as described (34). Briefly, frozen sections were fixed in precooled (−20 degrees C. for 10 mins at r.t., blocked with 10% goat serum for 15 mins, and stained with anti-COX IV (1:200 in 10% goat serum, 4 degrees C., overnight, Cat#: ab16056, Abcam) and labeled neuronal synapses with a-Bugarotoxin (0.5 μg/mL in 10% goat serum, r.t., 1 hour, Cat#: B-13423, Thermo Fisher Scientific).

COX/SDH double staining on 10 μm frozen gastrocnemius muscle sections used VitroView™ COX-SDH Double Histochemistry Stain Kit (Cat#: VB-3022, VitroVivo Biotech) according to the manufacturer's protocol.

Transmission electron microscopy and toluidine blue staining used standard techniques.

TUNEL staining on mice spinal cords used the DeadEnd Fluorometric TUNEL system (Cat#: G3250, Promega) according to the manufacturer's instructions. Briefly, lumbar spinal cords were fixed in 4% PFA overnight and embedded in paraffin before sectioning. After undergoing deparaffinization, slides were immersed in 0.1% TritonX-100 for 15 mins, washed twice with PBS, transferred to 100 μL Equilibration Buffer for 10 mins, and then reacted with 50 μL TdT reaction mix for 60 mins at 37 degrees C. The reaction was stopped with 2XSSC for 15 mins, followed by washing thrice with PBS. Anti-b-tubulin III staining was used to label neurons.

Mitochondrial respiration in reprogrammed ALS motor neurons was measured as the oxygen consumption rate (OCR) suing a Seahorse XFe24 Extracellular Flux Analyzer (Seahorse Bioscience, Billerica, MA, USA). Briefly, neurons were plated on the Seahorse XF24-well cell culture microplate (Cat#: 100777-004, Agilent), treated with Chimera or Compound 2A (100 nM) or DMSO vehicle and mitochondrial OCR measured 48 hours later. Before assays, sensor cartridges (Cat#: 102340-100, Agilent) were hydrated with XF calibrant (1 mL/well, Cat#: 100840-000, Aligent) in a non-CO₂ 37 degree C. incubator overnight. Neurons were washed 2 times in Seahorse XF assay DMEM medium (Cat#: 103680-100, Aligent) supplemented with 1 mM pyruvate (Cat#: 103578-100, Aligent), 2 mM glutamine (Cat#: 103579-100, Aligent) and 10 mM glucose (Cat#: 103577-100, Aligent), 500 μL assay medium was added after final wash, and the cells incubated in a non-CO₂ 37 degree C. incubator for 1 hour. After four basal respiration measurements, 1 μM oligomycin (inhibitor of ATP synthase), 1 μM FCCP (an optimized concentration to give maximum respiratory capacity), 0.5 μM rotenone/antimycin A (Seahorse XF Cell Mito Stress Test Assay, Cat#: 103010-100, Aligent) were autoinjected into the experimental wells. ATP-linked respiration is the decrease in oxygen consumption rate from basal respiration after injection of the ATP synthesis inhibitor oligomycin, data reported as basal OCR-post oligomycin OCR for each well. Each experimental column is an average of a minimum of 5 replicate wells and each experiment was performed with a minimum of three biological replicates.

Compound 2A toxicity was assessed in female 12 week old C57BL6/J mice (The Jackson Laboratory, Bar Harbor, Maine, USA, Stock #: 000664) that received 60 mg/kg Compound 2A in 10% DMSO/ 90% (30% HP-b-CD) or vehicle alone twice daily for 28 days via oral gavage. Cage-side clinical observations were made daily. At study termination on day 28 mice were sacrificed with an overdose of isoflurane followed by cervical dislocation and blood collected via left ventricular puncture.

Antioxidant capacity assays used total antioxidant capacity (TAC) assay kit (Cellbiolabs, Cat#: STA360), catalase activity assay kit (Cellbiolabs, Cat#: STA341) and superoxide dismutase activity assay (Cellbiolabs, Cat#: STA341) according to manufactural protocols. Compound 2A (1 μM) or DMSO was added to standard concentrations of uric acid, superoxide dismutase or catalase standard within a 96-well microtiter plate format. Samples and standards were diluted with the proper reaction reagent and, upon the addition of copper, hydrogen peroxide or xanthine solution/xanthine oxidase solution, reacted for 5 min or 1 hour as per manufacturer's instructions. The reactants were stopped and assayed using a 96-well spectrophotometric microplate reader at 490 nm or 520 nm.

### Statistics

Unless otherwise stated, data are reported as means±_SEM. Two-group comparisons used Student's t-test; multiple group comparisons used one-way ANOVA; time-course by treatment group or genotype by treatment group comparisons used two-way ANOVA with Tukey's posthoc test for individual statistical comparisons. P<0.05 was considered significant. The details of statistical methods, exact values of n and what n represents are indicated in figures and figure legends.

Mouse treatment was randomized according to a random integer table (even or odd) and performed by investigators blind to treatment status. Post terminal analysis of tissues was performed blindly.

### Example 1. Synthesis of Exemplary Compounds

### Compound 1

Synthesis of N-(trans-4-hydroxycyclohexyl)-5-phenylpentanamide (Compound 1). This mitofusin activator was prepared as described in U.S. Patent Application Publication 2020/0345668, which is incorporated herein by reference.

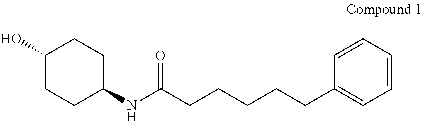 [SMILES: O=C(CCCCCc1ccccc1)N[C@H]1CC[C@H](O)CC1]

### Compound 2

Synthesis of N-(trans-4-hydroxycyclohexyl)-2-(3-phenylpropyl)cyclopropane-1-carboxamide (Compound 2)

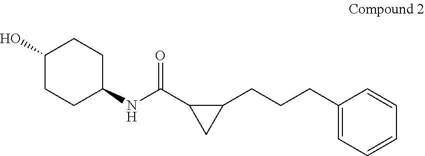 [SMILES: O=C(N[C@H]1CC[C@H](O)CC1)C1CC1CCCc1ccccc1]

Scheme 1 below outlines the synthesis of N-((1r,4r)-4-hydroxycyclohexyl)-2-(3-phenylpropyl)cyclopropane-1-carboxamide (Compound 2) in racemic form.

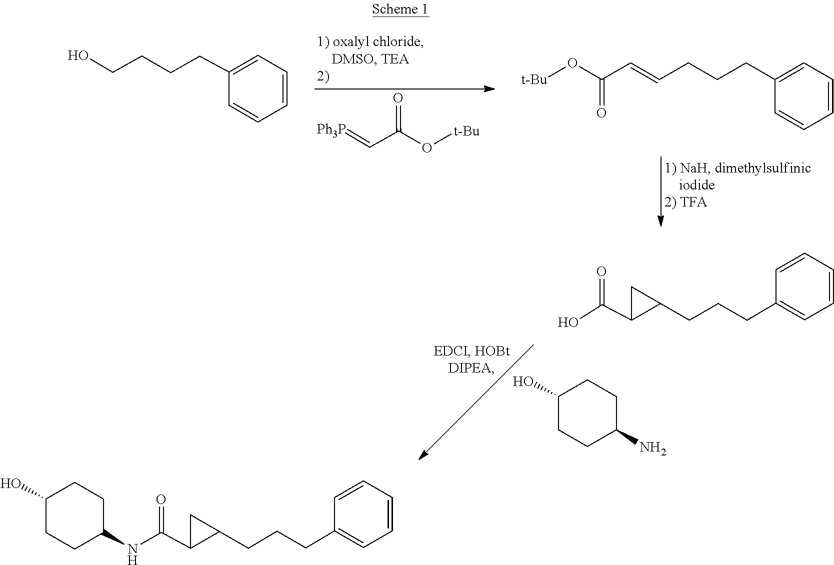

To a solution of oxalyl chloride (4.65 g, 36.6 mmol, 3.20 mL, 1.10 eq) in DCM (75.0 mL) cooled to −55° C. under N₂ atmosphere, a solution of DMSO (5.72 g, 73.2 mmol, 5.72 mL, 2.20 eq) in DCM (30.0 mL) was dropwise. After stirring for 5 min, 4-phenylbutan-1-ol was added dropwise (5.00 g, 33.2 mmol, 5.08 mL, 1.00 eq) in DCM (15.0 mL). After stirring for 15 min, TEA (16.8 g, 166 mmol, 23.1 mL, 5.00 eq) was added, and the reaction mixture was warmed to 25° C. To the warmed reaction mixture was then added 100 mL 1 N HCl, and the product was extracted with DCM 200 mL (100 mL×2). The combined organic layers were washed with water 50 mL, dried over Na₂SO_{4,} filtered and concentrated to give 4-phenylbutanal (5.00 g).

To a solution of 4-phenylbutanal (5.00 g, 33.7 mmol, 9.80 mL, 1.00 eq) in THF (50.0 mL) was added tert-butyl 2-(triphenyl-λ5-phosphaneylidene)acetate (16.5 g, 43.8 mmol, 1.30 eq). The reaction mixture was stirred at 20° C.for 12 hrs to give tert-butyl (E)-6-phenylhex-2-enoate (6.00 g, 24.3 mmol, 72.1% yield).

To a suspension of NaH (1.17 g, 29.2 mmol, 60.0% purity, 1.20 eq) in DMSO (30.0 mL) was added dimethylmethanesulfinic iodide (6.43 g, 29.2 mmol, 1.20 eq). The mixture was stirred at 20° C.for 0.5 hr, and tert-butyl (E)-6-phenylhex-2-enoate (6.00 g, 24.3 mmol, 1.00 eq) in DMSO (3.00 mL) was added. The reaction mixture was stirred at 20° C. for 1 hr to give tert-butyl 2-(3-phenylpropyl)cyclopropane-1-carboxylate (2.10 g, 8.07 mmol, 33.1% yield). Removal of the t-butyl ester was accomplished by adding TFA TFA (7.70 g, 67.5 mmol, 5.00 mL, 17.5 eq) to a solution of tert-butyl 2-(3-phenylpropyl)cyclopropane-1-carboxylate (1.00 g, 3.84 mmol, 1.00 eq) in DCM (5.00 mL). After stirring at 25° C. for 15 hrs, 2-(3-phenylpropyl)cyclopropane-1-carboxylic acid (800 mg) was obtained.

EDCI (1.00 g, 5.22 mmol, 1.50 eq), HOBt (564 mg, 4.18 mmol, 1.20 eq), DIPEA (1.35 g, 10.4 mmol, 1.82 mL, 3.00 eq), and trans-4-aminocyclohexan-1-ol (580 mg, 3.83 mmol, 1.10 eq. HCl) were added to a solution of 2-(3-phenylpropyl)cyclopropane-1-carboxylate (800 mg, 3.48 mmol, 1.00 eq) in DMF (8.00 mL) and stirred at 25 ° C.for 16 hrs. After solvent removal, the residue was purified by preparative HPLC (column, Waters Xbridge C18 150*50 mm*10 μm; mobile phase: [water (10 mM NH₄HCO₃)-ACN]; B%: 28%-58%, 11.5 min) to give the title compound as a white solid. LC-MS, Rₜ=0.904 min, m/z=302.1 (M+H)⁺. HPLC, Rₜ=2.898 min, purity: 98.6%, under 220 nm. ¹³C NMR: (400 MHZ, MeOD) δ 173.97, 142.27, 127.96, 127.89, 125.31, 69.07, 35.14, 33.45, 32.23, 30.87, 30.24, 21.27, 20.55, 13.04. ¹H NMR: (400 MHZ, MeOD) δ 7.27-7.24 (m, 2H), 7.18-7.15 (m, 3H), 3.64-3.61 (m, 1H), 3.55-3.50 (m, 1H), 2.64 (t, J=8 Hz, 2H), 1.97-1.89 (m, 4H), 1.75-1.73 (m, 2H), 1.36-1.04 (m, 8H), 1.29-1.27 (m, 1H), 0.59-0.57 (m, 1H).

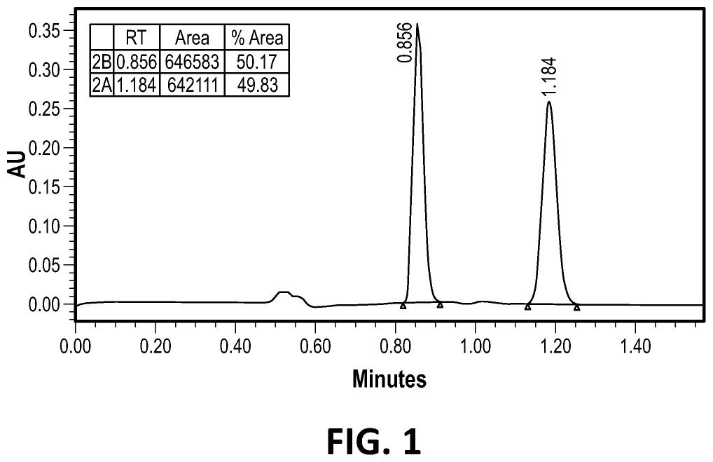

Chiral Separation of Compounds 2A and 2B. A Thar 200 preparative SFC (SFC-7) was used to separate Compounds 2A and 2B using a ChiralPak IG column (300×50 mm I.D., 10 μm) and the following mobile phase conditions: A for CO₂ and B for methanol (0.1% NH₃H₂O); gradient: B 35%, flow rate: 200 mL/min; back pressure: 100 bar; column temperature: 38° C.; wavelength: 220 nm; and cycle time: ˜ 4 min. Compound 2 was dissolved in ˜200 ml methanol and 10 mL injection volumes were used. After separation, the solvent was removed in vacuo at a bath temperature 40° C. to obtain each stereoisomer. Compound 2B eluted faster than did Compound 2A. FIG. 1 shows a representative HPLC chromatogram of the chiral separation of Compounds 2A and 2B. The trans stereochemistry of the cyclopropane ring was established based upon the known stereochemistry of the cyclopropanation reaction and the 19 Hz coupling constant of the cyclopropane ring protons. The absolute stereochemistry of each stereoisomer was established by x-ray crystallography, as discussed further below.

### Compound 4

Synthesis of (1R,2R)-2-((benzylthio)methyl)-N-((1r,4R)-4-hydroxycyclohexyl)cyclopropane-1-carboxamide (Compound 4) and Chiral Separation Thereof (Compounds 4A and 4B). The title compound was synthesized in a similar manner to that of Compound 2, except the starting material was phenylmethanethiol, which was reacted with 2-chloro-1,1-dimethoxyethane (1.2 eq) in ethanol in the presence of sodium ethoxide (1.0) eq) and KI (0.05 eq). The resulting benzyl(2,2-dimethoxyethyl)sulfane was stirred at 60° C. for 12 hours in H₂SO₄ to obtain 2-(benzylthio)acetaldehyde. The 2-(benzylthio)acetaldehyde was then further transformed following a series of reactions similar to those shown in Scheme 1. The title compound was purified as a racemic mixture by preparative HPLC using a Phenomenex Luna C18 column (250 mm*50 mm, 10 μm; mobile phase: [water (0.1% TFA)-ACN]; B%: 20%-50%, 20 min), and then purified by preparative SFC (column: DAICEL CHIRALPAK AD-H (250 mm*30 mm, 5 μm); mobile phase: [0.1% NH₃H₂O ETOH]; B%: 35%-35%, 2.7 min; 240 min). The product was purified by (column: Phenomenex Gemini-NX C18 75*30 mm*3 μm; mobile phase: [water (0.05% ammonium hydroxide v/v)-ACN]; B%: 15%-45%, 7 min) and (column: Phenomenex Gemini-NX C18 75*30 mm*3 μm; mobile phase: [water (0.225% FA)-ACN]; B%, 30%-60%, 2 min). The (R,R) and (S,S) stereoisomers of Compound 4 were obtained as separated peaks.

Compound 4A was 98.4% pure by HPLC and exhibited m/z=320.2 (M+H)⁺ by LCMS. Compound 4B was 97.3% pure by HPLC and exhibited m/z=319.9 (M+H)⁺ by LCMS. ¹H NMR, (Compound 4A) (400 MHZ MeOD) δ 7.33-7.21 (m, 5H), 3.76 (s, 2H), 3.60-3.57 (m, 1H), 3.52-3.50 (m, 1H), 2.44-2.40 (m, 2H), 1.94-1.89 (m, 4H), 1.42-1.40 (m, 2H), 1.33-1.29 (m, 4H), 1.08 (td, J=4.4, 8.8 Hz, 1H), 0.69 (ddd, J=4.2, 6.0, 8.4 Hz, 1H); ¹H NMR, (Compound 4B) (400 MHz MeOD) δ 7.32-7.28 (m, 5H), 3.76 (s, 2H), 3.61-3.58 (m, 1H), 3.52-3.50 (m, 1H), 2.44-2.40 (m, 2H), 1.94-1.89 (m, 4H), 1.43-1.41 (m, 2H), 1.33-1.28 (m, 4H), 1.08 (td, J=4.4, 8.8 Hz, 1H), 0.69 (ddd, J=4.2, 6.0, 8.4 Hz, 1H).

### Example 2. Mitofusin Activity and Pharmacokinetics of Exemplary Compounds.

Table 1A below summarizes the biological activity and pharmacokinetics of Compound 2 in comparison to Compound 1.

TABLE 1A

| | Compound 2 | Compound 1 | Compound 7 |
|---|---|---|---|
| EC₅₀ (nM), MFN2 | 5.1 | 5.8 | 5.6 |
| Plasma Protein | % Bound | % Bound | % Bound |
| Binding | | | |
| human | 94.4 | 91 | 89.1 |
| mouse | 95.5 | 96.3 | 93.1 |
| Liver Microsomes | T_{1/2} (min) | T_{1/2} (min) | T_{1/2} (min) |
| human | >145 | >145 | >145 |
| mouse | 114.1 | 92.4 | 76.7 |
| PAMPA assay | nm/s | nm/s | nm/s |
| | 58.451 | 26.277 | 39.0 |

| Pharmacokinetics | IV | PO | IV | PO |
|---|---|---|---|---|
| Dose (mg/kg) | 10 | 50 | 10 | 50 |
| Cₒ/Cₘₐₓ (ng/mL) | 32111 | 9900 | 50000 | 24000 |
| Tₘₐₓ (hr) | | 0.5 | | 0.5 |
| T_{1/2} (hr) | 1.64 | 3.02 | 1.1 | 2.2 |
| AUC (ng*hr/mL) | 9178 | 26163 | 11657 | 44025 |
| Vdss (L/kg) | 0.6 | | 0.347 | |
| % Bioavailability | | 59 | | 75.5 |

Testing details for the MFN activity and PAMPA assay are provided in U.S. Patent Applications 2020/034566 and 2020/0345669, incorporated herein by reference. Compound 2, compound 7, and Compound 1 exhibited similar EC₅₀ values toward mitofusin activation. Surprisingly, the PAMPA assay of Compound 2 exhibited over twice the value of Compound 1, which is characteristic of greater passive blood-brain barrier permeability. Further, Compound 2 exhibited a longer plasma half-life when administered IV or PO and a greater tissue distribution (Vdss).

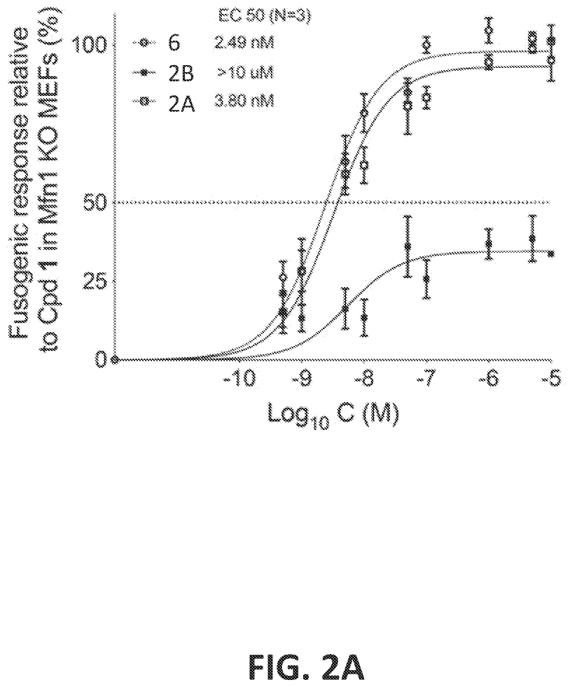

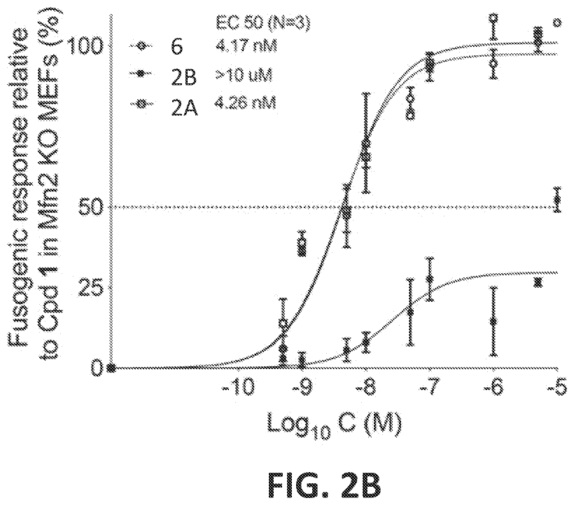

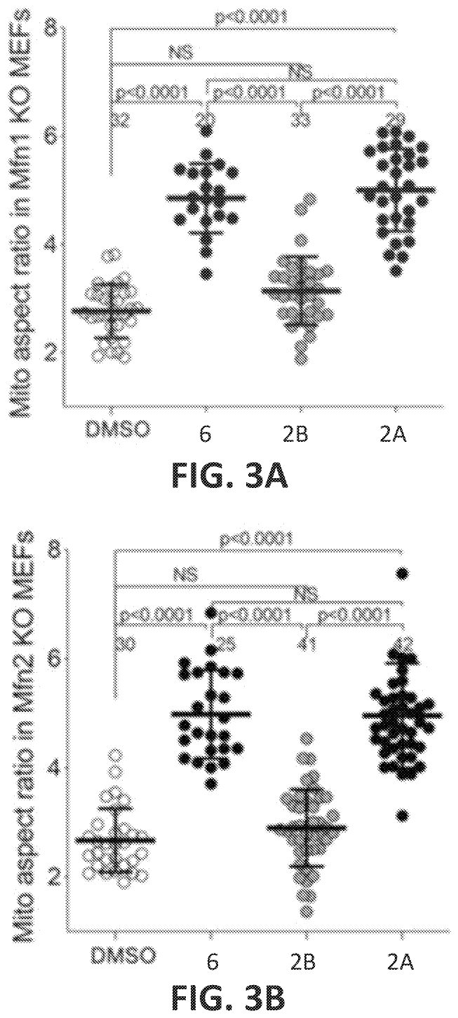

Biological testing of resolved stereoisomers of Compound 2 was conducted. Comparative mitofusin-stimulating activities of Compounds 2A and 2B were assayed as mitochondrial elongation and polarization status after 48-hour exposure in MFN1-null (i.e., expressing Mfn2 alone, MFN1 KO) or MFN2-null (i.e., expressing MFN1 only, MFN2 KO) murine embryonic fibroblasts (MEFs). FIGS. 2A and 2B show illustrative dose-response curves for Compounds 2A and 2B in comparison to Compound 6 for activity against MFN1 knockout MEFs and MFN2 knockout MEFs. As shown, Compound 2A exhibited a high activity comparable to that of Compound 6 in both assays. Compound 2B, in contrast, failed to even reach a response of 50% (EC₅₀>10 μm). FIGS. 3A and 3B show corresponding illustrative plots of mitochondrial aspect ratio obtained in the presence of Compounds 2A and 2B in comparison to Compound 6 and DMSO vehicle. Again, only Compound 2A was highly active in this assay.

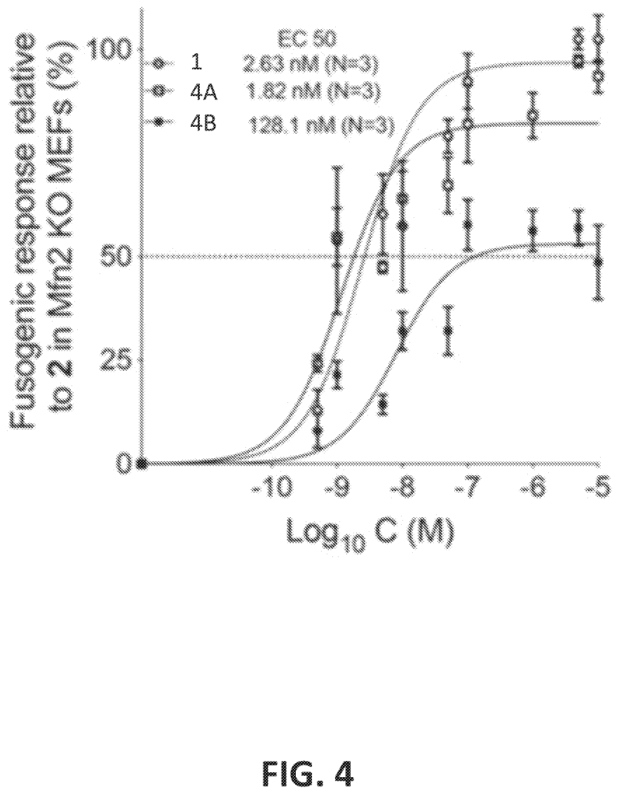

Dose-response curves for Compounds 4A and 4B were also determined. Although the EC₅₀ of Compound 4B was measurable in the sulfide case, Compound 4A was still considerably higher in activity. FIG. 4 shows dose-response curves for Compounds 4A and 4B in comparison to Compound 1 for activity against MFN2 knockout MEFs.

Thus, the slower-eluting stereoisomer was the more active compound in the case of both Compound 2A and its sulfide analogue (Compound 4A). Accordingly, the absolute stereochemistry of Compound 2A and Compound 4A were assigned by analogy to one another, as explained further below.

Given that Compound 2A represented the active stereoisomer of Compound 2, a more detailed pharmacokinetic study in plasma and brain tissue was performed for this compound. Table 2A summarizes the pharmacokinetic data. Compound 2A levels were simultaneously measured in plasma and brain tissue at increasing times after a single 50 mg/kg oral dose. Plasma pharmacokinetics after oral administration were similar to those of Compound 2 (mixture of stereoisomers) given at an identical dose and route in the same vehicle (10% DMSO, 90% [30% cyclodextrin]): tₘₐₓ for both was 0.5 hr, t_{1/2} was 2.83 hr and 3.02 hr respectively, and mean tissue residence times (MRT) were 3.96 hr and 3.58 hr respectively.

TABLE 2A

| | | | Actual Mean | | | | | |
|---|---|---|---|---|---|---|---|---|
| | | Dose | Dosage | t_{1/2} | tₘₐₓ | Cₘₐₓ | AUCₗₐₛₜ | MRT_{inf} |
| Compound | Tissue | Route | (mg/kg) | (h) | (h) | (ng/ml) | (h * ng/ml) | (h) |
| Compound | Plasma | PO | 50 | 2.83 | 0.50 | 12100 | 34000 | 3.96 |
| 2A | Brain | PO | 50 | 3.13 | 0.50 | 4030 | 13400 | 4.30 |
| | Spinal Cord | PO | 50 | 2.71 | 0.50 | 4060 | 12700 | 3.93 |
| | Left Sciatic | PO | 50 | 3.21 | 0.50 | 4570 | 15300 | 4.62 |
| | Nerve | | | | | | | |
| | Right Sciatic | PO | 50 | 2.61 | 0.50 | 5370 | 13800 | 3.35 |
| | Nerve | | | | | | | |

Compound 2A levels were measured in plasma and brain tissues at increasing times after a single 50 mg/kg oral dose. As reported in Table 2A, Compound 2A Cₘₐₓ, AUC, t_{1/2}, and mean residence time (MRT) were similar in all three neurological tissues. Accordingly, the above results suggested that Compound 2A might exhibit favorable nervous system pharmacodynamics.

Table 2B below summarizes the plasma and brain pharmacokinetics of Compound 1 in comparison to Compound 2A in fasting mice^{a}.

TABLE 2B

| | Compound 1 | | Compound 2A | |
|---|---|---|---|---|
| | total | free | total | free |
| | plasma | plasma | plasma | plasma |
| Cₘₐₓ (ng/mL) | 39 011 | 1428 | 33 619 | 1523 |
| t_{1/2} (h) | 1.280 | | 1.67 | |
| AUC₀₋ₗₐₛₜ | 30 612 | 1121 | 59 567 | 2697 |
| (ng · h/mL) | | | | |
| MRT₀₋ₗₐₛₜ (h) | 1.01 | | 1.92 | |
| | total brain | free brain | total brain | free brain |
| Cₘₐₓ (ng/g) | 10 001 | 970 | 13 480 | 774 |
| t_{1/2} (h) | 0.999 | | 1.77 | |
| AUC₀₋ₗₐₛₜ | 7392 | 717 | 23 465 | 1347 |
| (ng · h/g) | | | | |
| MRT₀₋ₗₐₛₜ (h) | 0.959 | | 1.98 | |

^{a}Free compound concentrations were calculated from protein binding assays: Compound 1, mouse plasma 96.7%, mouse brain 90.3%; Compound 2, mouse plasma 95.5%, mouse brain 94.3%.

A side-by-side comparison of Compound 1 and Compound 2A plasma and brain pharmacokinetics was performed. For these comparative studies, the two compounds were administered at the same dose (50 mg/kg) and route (oral gavage) and using the same vehicle (5 mg/mL in 30% SBE-bCD). As shown in Table 3, greater brain bioavailability (total and free AUCs) and longer plasma and brain t_{1/2s} and MRTs were exhibited by Compound 2A.

### Example 3. Mitofusin Activation Moderates Neuronal and Muscular Degeneration in SOD1G93A mice.

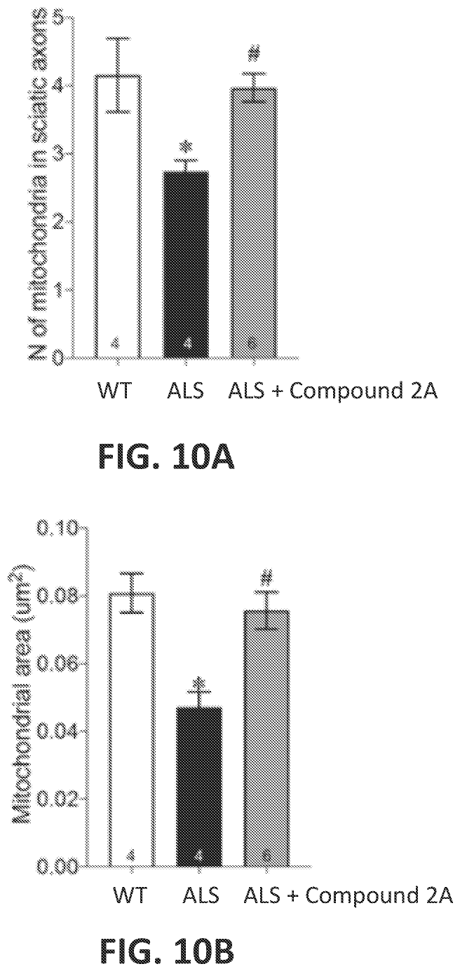

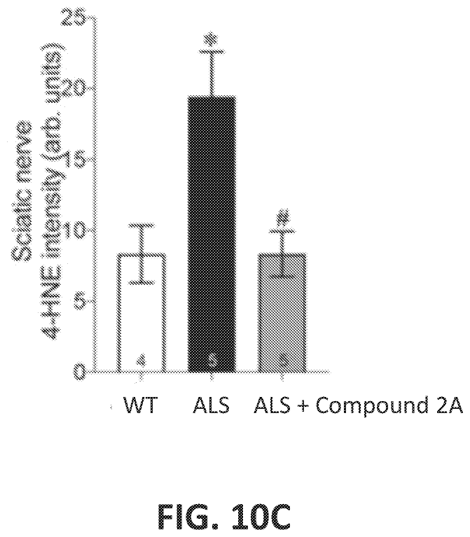

Number of mice evaluated per group is indicated at the base of bars in graphs (i.e. FIGS. 10-13).

Sustained mitofusin activation reversed mitochondrial abnormalities provoked by SOD1 G93A.

To understand mechanisms underlying protective effects of mitofusin activation SOD1G93A ALS mouse tissues were evaluated for hallmark mitochondrial, neuronal and skeletal myocyte phenotypes. Compound 2A increased mitochondrial number (FIG. 10A), improved mitochondrial fragmentation (FIG. 10B) and moderated mitochondrial cristae abnormalities (FIG. 10B) in ALS sciatic nerve axons examined at age 140 days. Moreover, mitochondriaderived ROS in ALS mouse sciatic nerve axons, which is increased as a consequence of the ALS SOD1 G93A mutation, decreased in Compound 2A-treated mice (FIG. 10C).

Sustained mitofusin activation moderated neuronal degeneration evoked by SOD1 G93A.

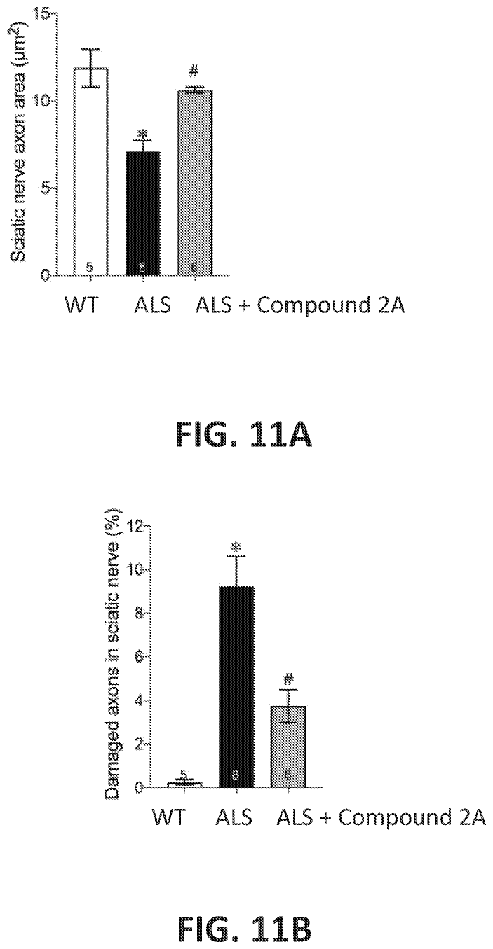

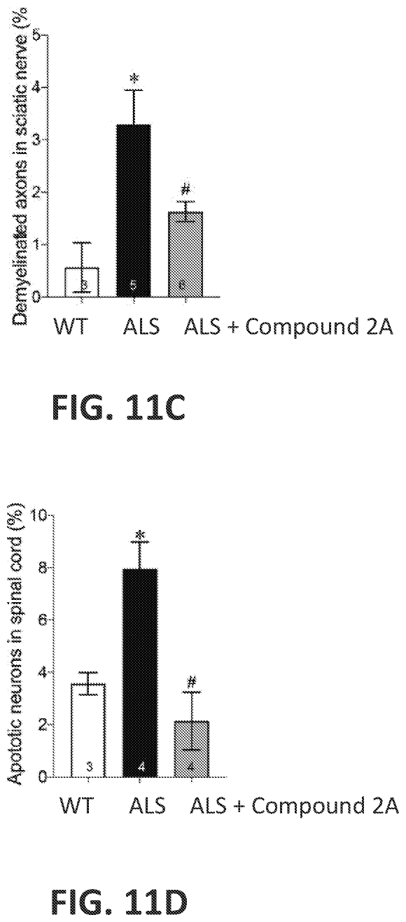

It is established that reduced mitochondrial injury correlated with neuromuscular protection in Compound 2A-treated ALS mice. Axons of ALS sciatic nerves exhibited less severe atrophy (i.e. larger axon diameter) and fewer myelin dense bodies with Compound 2A treatment (FIGS. 11A, 11B). Mitofusin activation also reduced the prevalence of apoptotic (TUNEL positive) neurons in the ventral horns of ALS mouse spinal cords (FIG. 11C).

Sustained mitofusin activation improved neuromuscular connectivity and reduced neurogenic muscular atrophy in SOD1G93A mice.

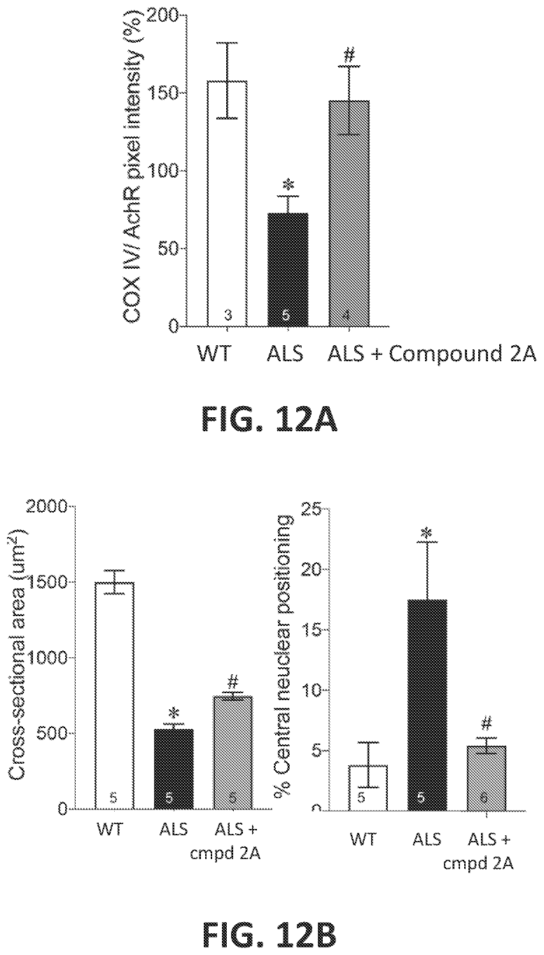

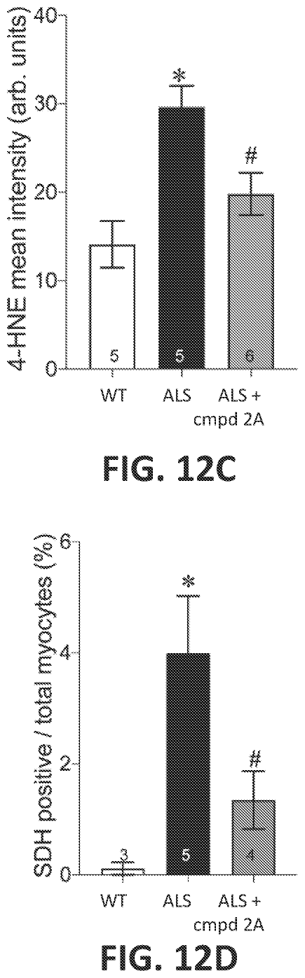

Mitochondrial residency within neuromuscular synapses at gastrocnemius muscles (that are innervated by affected sciatic nerves) was depressed in ALS and associated with myocyte atrophy and degenerative central myonuclear positioning. Each of these abnormalities was improved by Compound 2A treatment (FIGS. 12A-B). As in neuronal tissue, mitofusin activation suppressed ROS-induced protein damage (FIG. 12C), which in gastrocnemius muscle was linked to improved muscle oxidative capacity (SDH stain; FIG. 12D).

### Example 4. Mitofusin Activation Reduces Neuronal Mitotoxicity and Promotes Neuronal Growth in Cultured ALS Neurons

In vitro neuroprotective mechanisms evoked by mitofusin activation in SOD1 ALS.

In the context of established pathophysiology for SOD1 mutant ALS, the present disclosure suggests three possible disease-modulating mechanisms afforded by mitofusin activation:
1. Less mitotoxicity reduces neuronal death (neuroprotective effect);
2. Improved mitochondrial transport to neuronal termini improves neuronal repair and neuromuscular connectivity (neuroregenerative effect); and
3. Enhanced mitochondrial fitness reverses ALS-linked mitochondrial respiratory dysfunction (metabolic effect).

Each of these possibilities was examined in cultured ALS neurons.

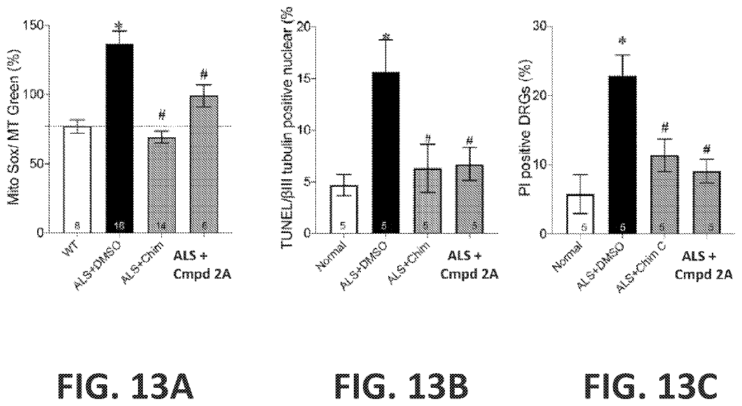

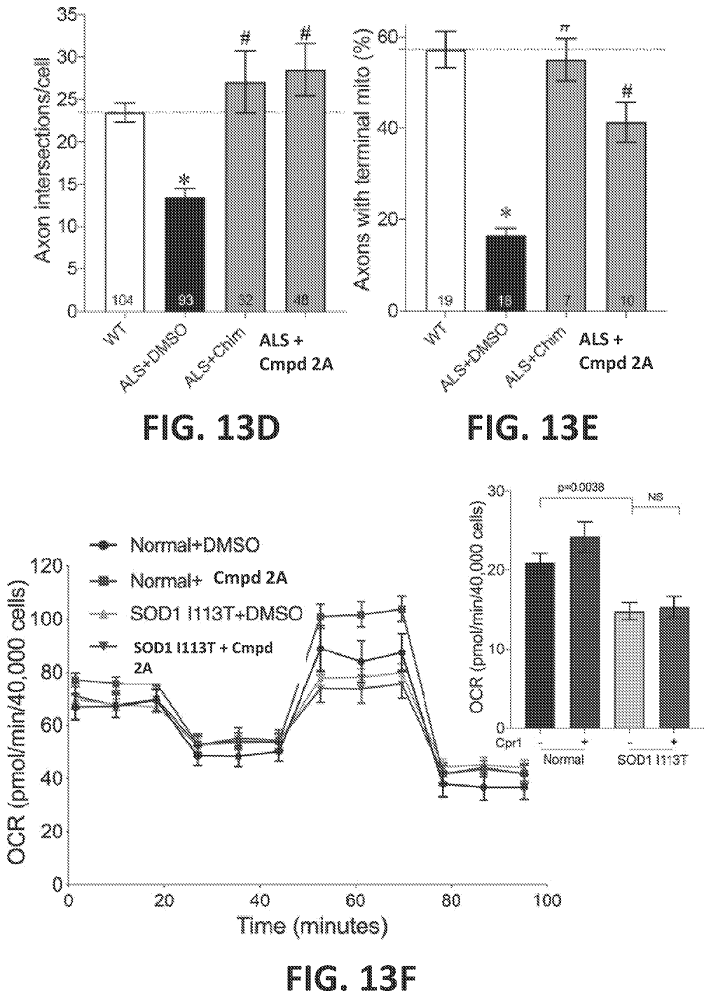

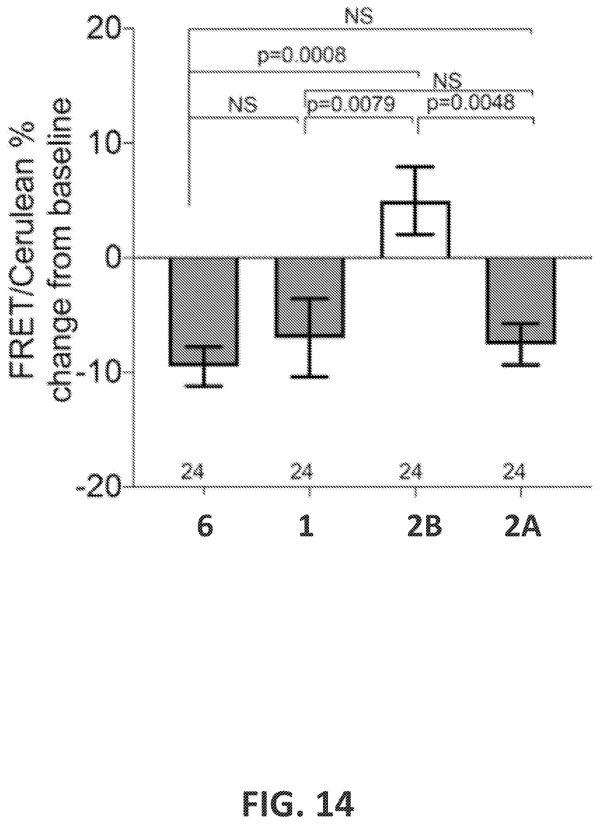

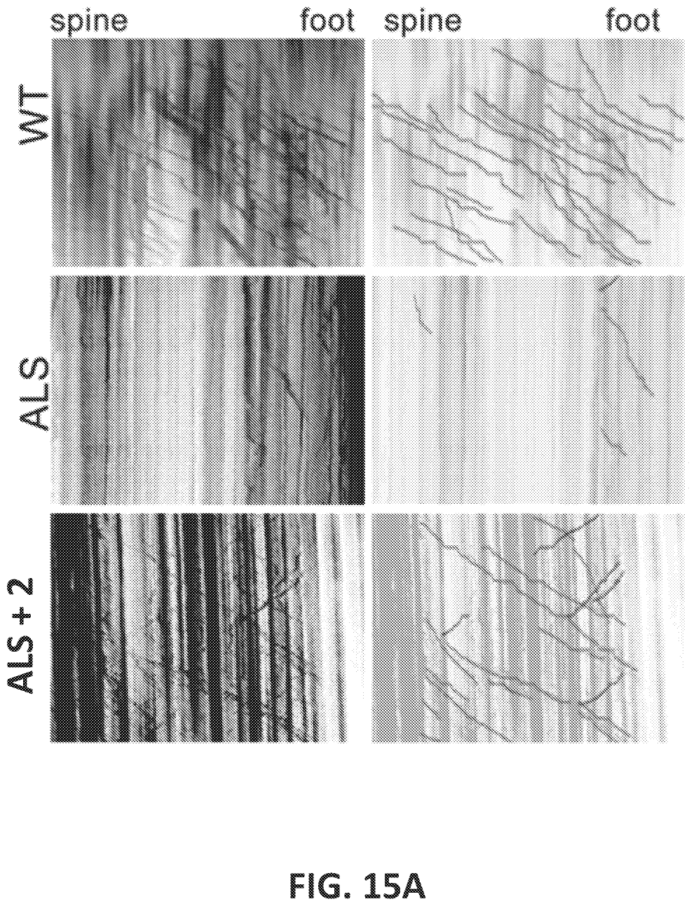

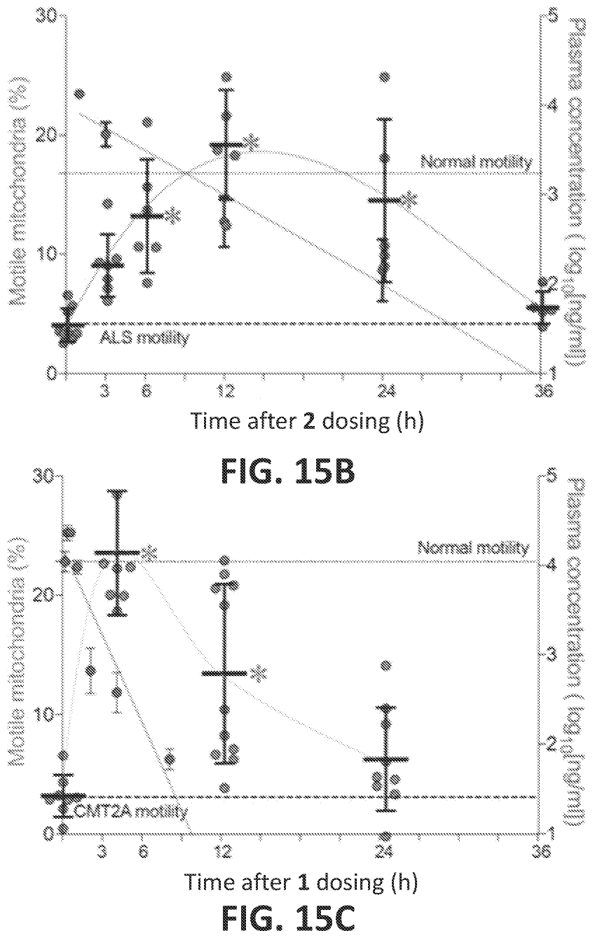

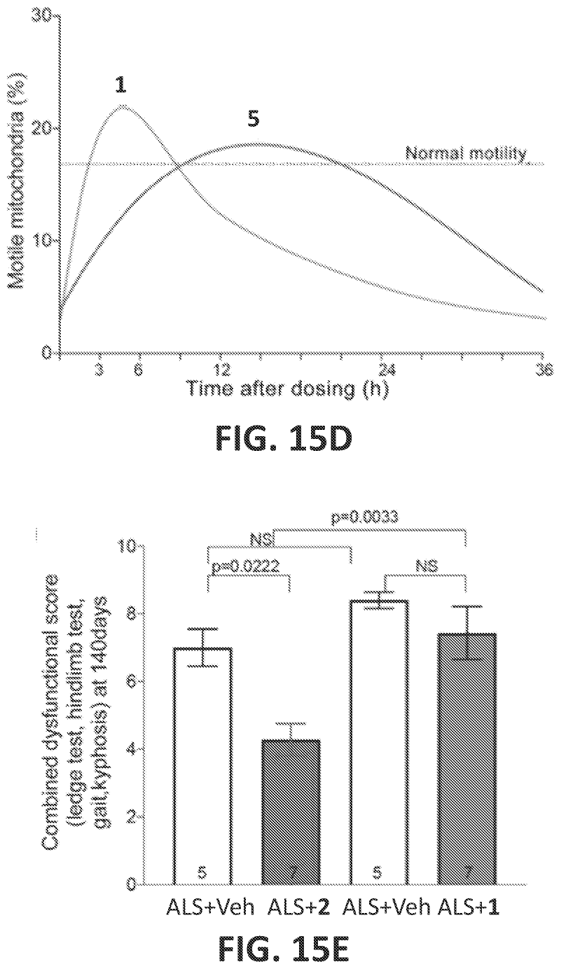

The effects of mitofusin activation on mitotoxicity (ROS elaboration) and associated neuronal death were interrogated in DRGs from SOD1 G93A mice. Effects of Chimera (a prototype small molecule mitofusin activator) and Compound 2A were evaluated in parallel. Each of these structurally diverse mitofusin activators suppressed mitochondrial ROS production (FIG. 13A) and reduced apoptotic (FIG. 13B) and necrotic (FIG. 13C) cell death that has a mitochondrial genesis in this disease. Both mitofusin activators also stimulated neuronal outgrowth while promoting mitochondrial localization to terminal growth buds (FIGS. 13D and 13E) ALS can exhibit characteristic metabolic abnormalities, which we also observed in Seahorse assays (FIG. 13F). Mitofusin activation did not improve mitochondrial metabolism in ALS neurons, measured either as oxygen consumption linked to ATP production (FIG. 13F, inset) or maximal oxygen consumption (FIG. 13F and not shown). Thus, activating mitofusins moderates preclinical ALS model through a combination of neuroprotective and neuroregenerative effects.

### Example 5. Characterization of Compounds 4A and 4B

Characterization of Compounds 4A and 4B by X-Ray Powder Diffraction, Crystal Growth, and Single Crystal X-Ray Crystallography. Compounds 4A and 4B were characterized crystallographically as surrogates to establish the absolute stereochemistry of Compounds 2A and 2B, respectively. In particular, the heavy sulfur atom was incorporated in these compounds to facilitate single-crystal x-ray crystallography studies.

X-Ray Powder Diffraction. As-obtained Compounds 4A and 4B exhibited a microcrystalline morphology when analyzed by x-ray powder diffraction. X-ray powder diffraction patterns were obtained on a Panalytical X′Pert Powder system on a Si zero-background sample holder. The 2θ position was calculated against a Pananalytical Si reference standard disc. Other experimental parameters are set forth in Table 3 below.

TABLE 3

| Parameters | Reflection Mode | Reflection Mode | Reflection Mode |
|---|---|---|---|
| X-Ray wavelength | Cu, kα | Cu, kα | Cu, kα |
| | Kα1 (Å): 1.540598, | Kα1 (Å): 1.540598, | Kα1 (Å): 1.540598, |
| | Kα2 (Å): 1.544426, | Kα2 (Å): 1.544426, | Kα2 (Å): 1.544426, |
| | Kα2/Kα1 intensity | Kα2/Kα1 intensity | Kα2/Kα1 intensity |
| | ratio: 0.50 | ratio: 0.50 | ratio: 0.50 |
| X-Ray tube setting | 45 kV, 40 mA | 45 kV, 40 mA | 45 kV, 40 mA |
| Divergence slit | Fixed ⅛° | Fixed ⅛° | Fixed ⅛° |
| Scan mode | Continuous | Continuous | Continuous |
| Scan range (° 2θ) | 3-40 | 3-40 | 3-40 |
| Scan step time [s] | 18.87 | 97.665 | 197.115 |
| Step size (° 2θ) | 0.0131 | 0.0263 | 0.0263 |
| Test Time | 4 min 15 s | 10 min 15 s | 20 min 15 s |

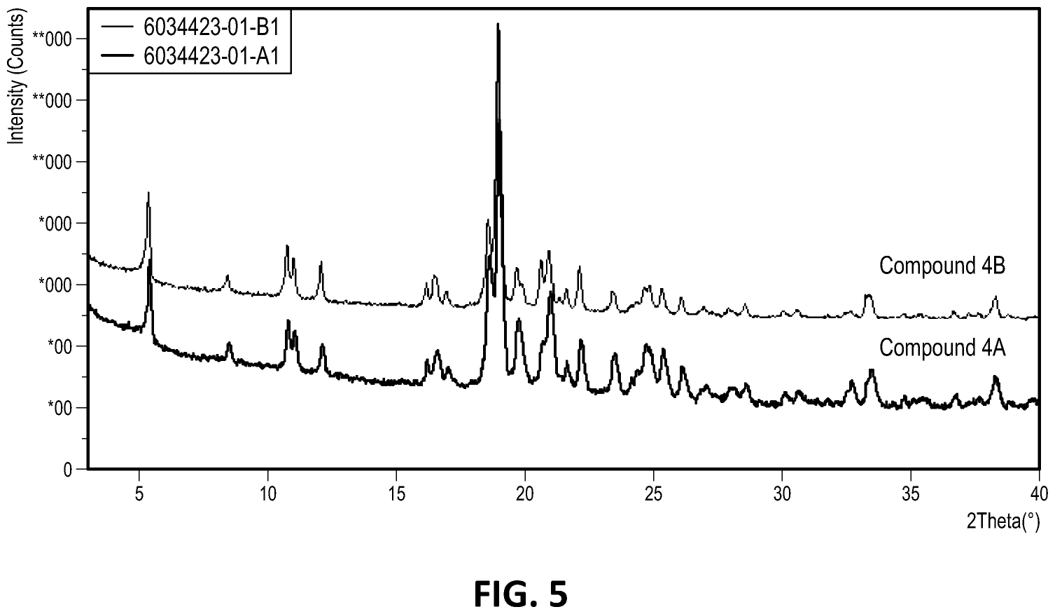

FIG. 5 is an illustrative x-ray powder diffraction pattern for Compounds 4A and 4B. As shown, the x-ray powder diffraction patterns for both stereoisomeric forms were substantially identical. Predominant peaks were found at the following approximate 2θ values: 5.41 (m), 8.48 (w), 10.42 (m), 10.79 (m), 12.10 (m), 16.20 (w), 16.49 (w), 16.99 (w), 18.33 (m), 18.96 (s), 19.72 (w), 20.64 (m), 20.96 (m), 21.64 (w), 22.13 (m), 23.45 (w), 24.68 (w), 24.87 (w), 25.34 (w), 26.10 (w), 33.27 (w), and 38.23 (w) (w=weak; m=medium; s=strong]).

Crystal Growth. Crystal growth experiments for Compounds 4A and 4B were attempted under a variety of conditions including slow evaporation, layer diffusion and slow cooling. For slow evaporation experiments, saturated solutions of Compounds 4A and 4B were placed in HPLC vials having perforated caps. Crystal growth was allowed to proceed at room temperature. Samples not providing crystals under these conditions were attempted under slow cooling conditions. Slow cooling was conducted by slurrying the sample at 35-60° C. in the indicated solvent, filtering through a 0.2 mm PTFE membrane, and cooling the solution to 5° C. at a ramp rate of 0.1° C./min. Tables 4 and 5 summarize the slow evaporation and slow cooling crystallization results, respectively. Samples marked with an asterisk in Table 4 afforded crystals before slow cooling could be conducted.

TABLE 4

| Experiment ID | Solvent (v:v) | Results |
|---|---|---|
| Compound 4B-1 | MeOH | Crystals |
| Compound 4B-2 | EtOH | Clear* |
| Compound 4B-3 | IPA | Moved to slow cooling |
| Compound 4B-4 | THF | Moved to slow cooling |
| Compound 4B-5 | CH₃Cl | Moved to slow cooling |
| Compound 4B-6 | DCM | Moved to slow cooling |
| Compound 4B-7 | Dioxane | Crystals |
| Compound 4B-8 | ACN | Crystals |
| Compound 4B-9 | 2-MeTHF | Moved to slow cooling |
| Compound 4B-10 | Acetone | Crystals |
| Compound 4A-1 | MeOH | Clear* |
| Compound 4A-2 | EtOH | Clear* |
| Compound 4A-3 | IPA | Moved to slow cooling |
| Compound 4A-4 | THF | Moved to slow cooling |
| Compound 4A-5 | CH₃Cl | Moved to slow cooling |
| Compound 4A-6 | DCM | Moved to slow cooling |
| Compound 4A-7 | ACN | Moved to slow cooling |
| Compound 4A-8 | MEK | Moved to slow cooling |
| Compound 4A-9 | MIBK | Moved to slow cooling |
| Compound 4A-10 | EtOAc | Crystals |

TABLE 5

| | | Heated | |
|---|---|---|---|
| Experiment ID | Solvent (v:v) | Temperature | Results |
| Compound 4B-4 | THF | 60° C. | Clear |
| Compound 4B-5 | CH3Cl | 60° C. | Crystals |
| Compound 4B-6 | DCM | 35° C. | Clear |
| Compound 4B-7 | Dioxane | 60° C. | Crystals |
| Compound 4B-8 | ACN | 60° C. | Crystals |
| Compound 4B-9 | 2-MeTHF | 60° C. | Crystals |
| Compound 4B-10 | Acetone | 50° C. | Crystals |
| Compound 4A-3 | IPA | 60° C. | Clear |
| Compound 4A-4 | THF | 60° C. | Clear |
| Compound 4A-5 | CH3Cl | 60° C. | Clear |
| Compound 4A-6 | DCM | 35° C. | Clear |
| Compound 4A-7 | ACN | 60° C. | Crystals |
| Compound 4A-8 | MEK | 60° C. | Crystals |
| Compound 4A-9 | MIBK | 60° C. | Crystals |
| Compound 4A-10 | EtOAc | 60° C. | Crystals |

Layer diffusion crystallization experiments were conducted by placing a saturated solution of Compounds 4A or 4B in an HPLC vial and carefully layering an anti-solvent on top of the saturated solution. The vials were then left at room temperature to allow the two solvents to diffuse into one another. Table 6 summarizes the layer diffusion crystallization experiments.

TABLE 6

| Experiment ID | Solvent | Anti-Solvent | Results |
|---|---|---|---|
| Compound 4B-1 | DCM | MTBE | Crystals |
| Compound 4B-2 | DCM | n-Heptane | Clear |
| Compound 4B-3 | DCM | Toluene | Clear |
| Compound 4B-4 | DMSO | H₂O | Crystals |
| Compound 4B-5 | DMSO | MTBE | Clear |
| Compound 4B-6 | DMSO | IPAc | Clear |
| Compound 4B-7 | DMSO | Toluene | Clear |
| Compound 4B-8 | THF | n-Heptane | Crystals |
| Compound 4B-9 | THF | MTBE | Clear |
| Compound 4B-10 | EtOH | n-Heptane | Clear |
| Compound 4A-1 | DCM | MTBE | Crystals |
| Compound 4A-2 | DCM | n-Heptane | Clear |
| Compound 4A-3 | DCM | Toluene | Clear |
| Compound 4A-4 | DMSO | H₂O | Crystals |
| Compound 4A-5 | DMSO | MTBE | Clear |
| Compound 4A-6 | DMSO | IPAc | Clear |
| Compound 4A-7 | DMSO | Toluene | Clear |
| Compound 4A-8 | THF | n-Heptane | Clear |
| Compound 4A-9 | THF | MTBE | Clear |
| Compound 4A-10 | EtOH | n-Heptane | Clear |

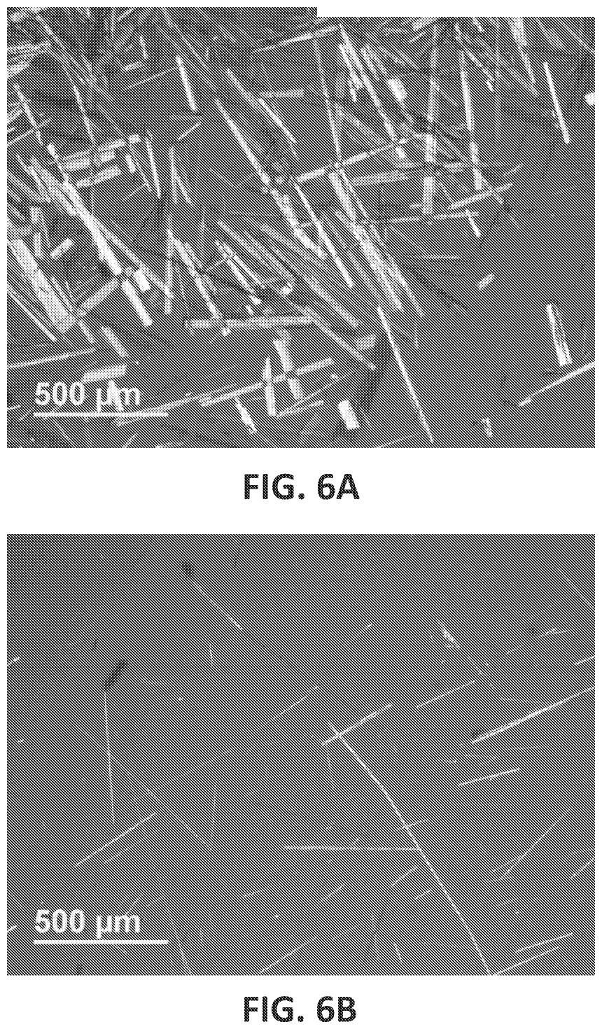

Single-Crystal X-Ray Diffraction. Single crystals of Compound 4A were obtained as rods from slow evaporation in ethyl acetate (Table 4, Compound 4A-10). Single crystals of Compound 4B were obtained as needles from slow evaporation in acetonitrile (Table 4, Compound 4B-8). FIGS. 6A and 6B show illustrative polarized light microscopy images of crystals of Compounds 4A and 4B, respectively.

Each sample was mounted on a MiTeGen mylar MicroLoop™ in a random orientation and immersed in a low viscosity cryo-oil (MiTeGen LV5 CryoOil™) and placed within a liquid nitrogen stream at 173 K controlled by an Oxford 800 CryoStream cooling system.

X-ray intensity data were measured on a Bruker D8 VENTURE (IμS microfocus X-ray source, Cu Kα, λ=1.54178 Å, PHOTON CMOS detector) diffractometer. The strategy was created and optimized with the Bruker Apex3 software, and the frames were integrated with the Bruker SAINT software package. Integration of the data using a monoclinic unit cell yielded a total of 22379 reflections to a maximum θ angle of 67.679° (0.83 Å resolution), of which 3511 were independent (Rᵢₙₜ=6.73%, R_{sig}=3.92%) and were greater than 2σ(F²). The final cell constants of a=10.556(9) Å, b=4.991(2) Å, c=16.855(13) Å, α=γ=90°, β=102.89(3)°, cell volume=865.6(11) Å³, are based upon the refinement of the XYZ-centroids of 3511 reflections above 20 σ(I) with 2.689°<θ<74.849°. Data were corrected for absorption effects using the Multi-Scan method (SADABS-2016/2). The absorption coefficient μ of this material is 1.706 mm⁻¹ at this wavelength (1.54178 Å). The calculated minimum and maximum transmission coefficients (based on crystal size) are 0.7946 and 1.000. The agreement factor for the averaging was 3.69% based on intensity.

Table 7 summarizes the single-crystal x-ray crystallographic data of Compound 4A. Tables 8-10 below provide a listing of atomic coordinates and other crystallographic data for Compound 4A.

TABLE 7

| | |
|---|---|
| Empirical formula | C₁₈H₂₅NO₂S |
| Formula weight | 319.45 |
| Temperature | 173(2) K |
| Wavelength | 1.54178 Å |
| Crystal system | Monoclinic |
| Space group | P2₁ |
| Unit cell dimensions | a = 10.556(9) Å α = 90° |
| | b = 4.991(2) Å β = 102.89(3)° |
| | c = 16.855(13) Å γ = 90° |
| Volume | 865.6(11) Å³ |
| Z | 2 |
| Density (calculated) | 1.226 Mg/m³ |
| Absorption coefficient | 1.706 mm⁻¹ |
| F(000) | 344 |
| Crystal size | 0.331 × 0.051 × 0.045 mm³ |
| Theta range for data collection | 2.689 to 74.849°. |
| Index ranges | −13 <= h <= 13, −6 <= k <= 5, |
| | −21 <= 1 <= 21 |
| Reflections collected | 22379 |
| Independent reflections | 3511 [R(int) = 0.0673] |
| Completeness to theta = 67.679° | 100.0% |
| Absorption correction | Semi-empirical from equivalents |
| Max. and min. transmission | 1.000 and 0.7946 |
| Refinement method | Full-matrix least-squares on F² |
| Data/restraints/parameters | 3511/1/199 |
| Goodness-of-fit on F² | 1.034 |
| Final R indices [I > 2sigma(I)] | R1 = 0.0369, wR² = 0.0883 |
| R indices (all data) | R1 = 0.0451, wR² = 0.0938 |
| Absolute structure parameter | 0.056(12) |
| Extinction coefficient | n/a |
| Largest diff. peak and hole | 0.164 and −0.256 e · Å⁻³ |

Atomic Coordinates presented as (x104) and displacement parameters in (Å²×10³). U(eq) is defined as one third of the trace of the orthogonalized U^{ij} tensor.

TABLE 8

| | x | y | z | U(eq) |
|---|---|---|---|---|
| O(1) | 4920(3) | 7546(4) | 9653(1) | 55(1) |
| C(2) | 5106(3) | 8206(6) | 8860(2) | 37(1) |
| C(3) | 4688(3) | 5828(6) | 8309(2) | 39(1) |
| C(4) | 4934(3) | 6338(6) | 7461(2) | 34(1) |
| C(5) | 6344(3) | 7057(5) | 7502(2) | 28(1) |
| C(6) | 6744(3) | 9472(5) | 8049(2) | 32(1) |
| C(7) | 6504(3) | 8958(6) | 8895(2) | 38(1) |
| N(8) | 6594(2) | 7605(4) | 6701(1) | 28(1) |
| C(9) | 6770(3) | 5650(5) | 6192(2) | 28(1) |
| O(10) | 6762(2) | 3256(4) | 6373(1) | 41(1) |
| C(11) | 6973(3) | 6534(6) | 5389(2) | 31(1) |
| C(12) | 6291(3) | 4898(7) | 4663(2) | 40(1) |
| C(13) | 7722(3) | 4670(6) | 4952(2) | 31(1) |
| C(14) | 8592(3) | 5792(6) | 4441(2) | 40(1) |
| S(15) | 8556(1) | 3480(2) | 3604(1) | 46(1) |
| C(16) | 9615(4) | 5159(9) | 3058(2) | 59(1) |
| C(17) | 9477(3) | 3854(8) | 2237(2) | 47(1) |
| C(18) | 10234(3) | 1699(8) | 2127(2) | 51(1) |
| C(19) | 10132(4) | 546(8) | 1374(2) | 58(1) |
| C(20) | 9262(4) | 1507(9) | 716(2) | 60(1) |
| C(21) | 8474(4) | 3603(11) | 806(2) | 66(1) |
| C(22) | 8574(4) | 4791(9) | 1564(2) | 59(1) |

Bond lengths [Å] and angles [°] for 6034423_03_B10-FF.

TABLE 9

| | |
|---|---|
| O(1)—C(2) | 1.432(3) |
| O(1)—H(1A) | 0.8400 |
| C(2)—C(7) | 1.511(5) |
| C(2)—C(3) | 1.511(4) |
| C(2)—H(2A) | 1.0000 |
| C(3)—C(4) | 1.529(4) |
| C(3)—H(3A) | 0.9900 |
| C(3)—H(3B) | 0.9900 |
| C(4)—C(5) | 1.517(4) |
| C(4)—H(4A) | 0.9900 |
| C(4)—H(4B) | 0.9900 |
| C(5)—N(8) | 1.457(3) |
| C(5)—C(6) | 1.519(4) |
| C(5)—H(5A) | 1.0000 |
| C(6)—C(7) | 1.526(4) |
| C(6)—H(6A) | 0.9900 |
| C(6)—H(6B) | 0.9900 |
| C(7)—H(7A) | 0.9900 |
| C(7)—H(7B) | 0.9900 |
| N(8)—C(9) | 1.339(3) |
| N(8)—H(8A) | 0.8800 |
| C(9)—O(10) | 1.234(4) |
| C(9)—C(11) | 1.484(4) |
| C(11)—C(12) | 1.513(4) |
| C(11)—C(13) | 1.515(4) |
| C(11)—H(11A) | 1.0000 |
| C(12)—C(13) | 1.485(5) |
| C(12)—H(12A) | 0.9900 |
| C(12)—H(12B) | 0.9900 |
| C(13)—C(14) | 1.500(4) |
| C(13)—H(13A) | 1.0000 |
| C(14)—S(15) | 1.816(3) |
| C(14)—H(14A) | 0.9900 |
| C(14)—H(14B) | 0.9900 |
| S(15)—C(16) | 1.805(4) |
| C(16)—C(17) | 1.507(5) |
| C(16)—H(16A) | 0.9900 |
| C(16)—H(16B) | 0.9900 |
| C(17)—C(18) | 1.377(5) |
| C(17)—C(22) | 1.390(5) |
| C(18)—C(19) | 1.376(5) |
| C(18)—H(18A) | 0.9500 |
| C(19)—C(20) | 1.359(6) |
| C(19)—H(19A) | 0.9500 |
| C(20)—C(21) | 1.365(7) |
| C(20)—H(20A) | 0.9500 |
| C(21)—C(22) | 1.392(6) |
| C(21)—H(21A) | 0.9500 |
| C(22)—H(22A) | 0.9500 |
| C(2)—O(1)—H(1A) | 109.5 |
| O(1)—C(2)—C(7) | 110.9(3) |
| O(1)—C(2)—C(3) | 108.1(2) |
| C(7)—C(2)—C(3) | 111.5(2) |
| O(1)—C(2)—H(2A) | 108.7 |
| C(7)—C(2)—H(2A) | 108.7 |
| C(3)—C(2)—H(2A) | 108.7 |
| C(2)—C(3)—C(4) | 110.9(2) |
| C(2)—C(3)—H(3A) | 109.5 |
| C(4)—C(3)—H(3A) | 109.5 |
| C(2)—C(3)—H(3B) | 109.5 |
| C(4)—C(3)—H(3B) | 109.5 |
| H(3A)—C(3)—H(3B) | 108.0 |
| C(5)—C(4)—C(3) | 111.5(3) |
| C(5)—C(4)—H(4A) | 109.3 |
| C(3)—C(4)—H(4A) | 109.3 |
| C(5)—C(4)—H(4B) | 109.3 |
| C(3)—C(4)—H(4B) | 109.3 |
| H(4A)—C(4)—H(4B) | 108.0 |
| N(8)—C(5)—C(4) | 112.4(2) |
| N(8)—C(5)—C(6) | 109.2(2) |
| C(4)—C(5)—C(6) | 110.6(2) |
| N(8)—C(5)—H(5A) | 108.1 |
| C(4)—C(5)—H(5A) | 108.1 |
| C(6)—C(5)—H(5A) | 108.1 |
| C(5)—C(6)—C(7) | 110.6(2) |
| C(5)—C(6)—H(6A) | 109.5 |
| C(7)—C(6)—H(6A) | 109.5 |
| C(5)—C(6)—H(6B) | 109.5 |
| C(7)—C(6)—H(6B) | 109.5 |
| H(6A)—C(6)—H(6B) | 108.1 |
| C(2)—C(7)—C(6) | 111.7(2) |
| C(2)—C(7)—H(7A) | 109.3 |
| C(6)—C(7)—H(7A) | 109.3 |
| C(2)—C(7)—H(7B) | 109.3 |
| C(6)—C(7)—H(7B) | 109.3 |
| H(7A)—C(7)—H(7B) | 107.9 |
| C(9)—N(8)—C(5) | 122.4(2) |
| C(9)—N(8)—H(8A) | 118.8 |
| C(5)—N(8)—H(8A) | 118.8 |
| O(10)—C(9)—N(8) | 122.6(2) |
| O(10)—C(9)—C(11) | 121.6(2) |
| N(8)—C(9)—C(11) | 115.9(2) |
| C(9)—C(11)—C(12) | 115.9(2) |
| C(9)—C(11)—C(13) | 117.3(2) |
| C(12)—C(11)—C(13) | 58.7(2) |
| C(9)—C(11)—H(11A) | 117.3 |
| C(12)—C(11)—H(11A) | 117.3 |
| C(13)—C(11)—H(11A) | 117.3 |
| C(13)—C(12)—C(11) | 60.69(19) |
| C(13)—C(12)—H(12A) | 117.7 |
| C(11)—C(12)—H(12A) | 117.7 |
| C(13)—C(12)—H(12B) | 117.7 |
| C(11)—C(12)—H(12B) | 117.7 |
| H(12A)—C(12)—H(12B) | 114.8 |
| C(12)—C(13)—C(14) | 119.4(3) |
| C(12)—C(13)—C(11) | 60.6(2) |
| C(14)—C(13)—C(11) | 120.2(3) |
| C(12)—C(13)—H(13A) | 115.2 |
| C(14)—C(13)—H(13A) | 115.2 |
| C(11)—C(13)—H(13A) | 115.2 |
| C(13)—C(14)—S(15) | 107.4(2) |
| C(13)—C(14)—H(14A) | 110.2 |
| S(15)—C(14)—H(14A) | 110.2 |
| C(13)—C(14)—H(14B) | 110.2 |
| S(15)—C(14)—H(14B) | 110.2 |
| H(14A)—C(14)—H(14B) | 108.5 |
| C(16)—S(15)—C(14) | 101.13(16) |
| C(17)—C(16)—S(15) | 108.9(3) |
| C(17)—C(16)—H(16A) | 109.9 |
| S(15)—C(16)—H(16A) | 109.9 |
| C(17)—C(16)—H(16B) | 109.9 |
| S(15)—C(16)—H(16B) | 109.9 |
| H(16A)—C(16)—H(16B) | 108.3 |
| C(18)—C(17)—C(22) | 117.8(3) |
| C(18)—C(17)—C(16) | 121.4(4) |
| C(22)—C(17)—C(16) | 120.8(4) |
| C(19)—C(18)—C(17) | 121.5(4) |
| C(19)—C(18)—H(18A) | 119.2 |
| C(17)—C(18)—H(18A) | 119.2 |
| C(20)—C(19)—C(18) | 120.3(4) |
| C(20)—C(19)—H(19A) | 119.9 |
| C(18)—C(19)—H(19A) | 119.9 |
| C(19)—C(20)—C(21) | 119.9(4) |
| C(19)—C(20)—H(20A) | 120.1 |
| C(21)—C(20)—H(20A) | 120.1 |
| C(20)—C(21)—C(22) | 120.3(4) |
| C(20)—C(21)—H(21A) | 119.9 |
| C(22)—C(21)—H(21A) | 119.9 |
| C(17)—C(22)—C(21) | 120.3(4) |
| C(17)—C(22)—H(22A) | 119.9 |
| C(21)—C(22)—H(22A) | 119.9 |

Anisotropic displacement parameters are provided in Table 11 and given in Å²×10³, and the factor exponent takes the form: −2π²[h²a*²U¹¹+ . . . +2 h k a* b* U^{12]}

TABLE 11

| | U¹¹ | U²² | U³³ | U²³ | U¹³ | U¹² |
|---|---|---|---|---|---|---|
| O(1) | 111(2) | 26(1) | 43(1) | 1(1) | 49(1) | −3(1) |
| C(2) | 65(2) | 20(1) | 35(1) | 4(1) | 28(1) | 2(1) |
| C(3) | 58(2) | 24(2) | 43(2) | −1(1) | 28(1) | −5(1) |
| C(4) | 42(2) | 28(1) | 35(2) | −1(1) | 14(1) | −3(1) |
| C(5) | 42(2) | 18(1) | 25(1) | 0(1) | 12(1) | 2(1) |
| C(6) | 45(2) | 21(1) | 34(2) | −3(1) | 17(1) | −2(1) |
| C(7) | 58(2) | 27(2) | 29(1) | −2(1) | 13(1) | 4(1) |
| N(8) | 45(1) | 17(1) | 25(1) | 1(1) | 13(1) | −1(1) |
| C(9) | 38(1) | 19(1) | 28(1) | −1(1) | 10(1) | −2(1) |
| O(10) | 74(1) | 19(1) | 35(1) | 2(1) | 24(1) | −2(1) |
| C(11) | 46(2) | 21(1) | 28(1) | 0(1) | 14(1) | −1(1) |
| C(12) | 44(2) | 48(2) | 30(1) | −4(1) | 12(1) | −5(1) |
| C(13) | 42(2) | 25(1) | 30(1) | −1(1) | 15(1) | 0(1) |
| C(14) | 48(2) | 37(2) | 38(2) | −2(1) | 20(1) | −6(1) |
| S(15) | 62(1) | 41(1) | 44(1) | −7(1) | 29(1) | −10(1) |
| C(16) | 64(2) | 70(3) | 53(2) | −16(2) | 33(2) | −23(2) |
| C(17) | 50(2) | 58(2) | 39(2) | −4(2) | 22(1) | −15(2) |
| C(18) | 44(2) | 65(2) | 47(2) | 1(2) | 11(2) | −9(2) |
| C(19) | 53(2) | 61(2) | 63(2) | −14(2) | 21(2) | −7(2) |
| C(20) | 74(3) | 66(3) | 44(2) | −9(2) | 21(2) | −14(2) |
| C(21) | 76(3) | 73(3) | 45(2) | 11(2) | 4(2) | −7(2) |
| C(22) | 64(2) | 54(2) | 62(2) | 2(2) | 20(2) | 3(2) |

The structure was solved with the ShelXT structure solution program using Intrinsic Phasing and refined with ShelXL (Version 2014/7) refinement package using full-matrix least-squares on F² contained in the SHELX software suite using the space group P4₃ with Z=2 for the formula unit, C₁₈H₂₅NO₂S). All non-hydrogen atoms were refined anisotropically. The positions of the hydrogen atoms connected to carbon atoms were geometrically idealized and refined using the riding model. The final anisotropic full-matrix least-squares refinement on F² with 199 parameters variables converged at R₁=3.69%, for the observed data and wR₂=9.38% for all data. The goodness-of-fit was 1.034. The largest peak in the final difference electron density synthesis was 0.164 e⁻/Å³ and the largest hole was −0.256 e⁻/Å³. Based on the final model, the calculated density is 1.226 g/cm³ and F (000), 1140 e⁻. The Absolute Structure Parameter (Flack(x) factor) refined to a value of 0.056(12), and the statistical analysis of Bivjoet Pairs (Hooft(y) factor) refined to 0.058(10), indicating that the molecule's absolute stereochemistry was determined with statistical significance. This was further corroborated with a TWIN/BASF refinement, which inferred no enantiomeric twinning was present.

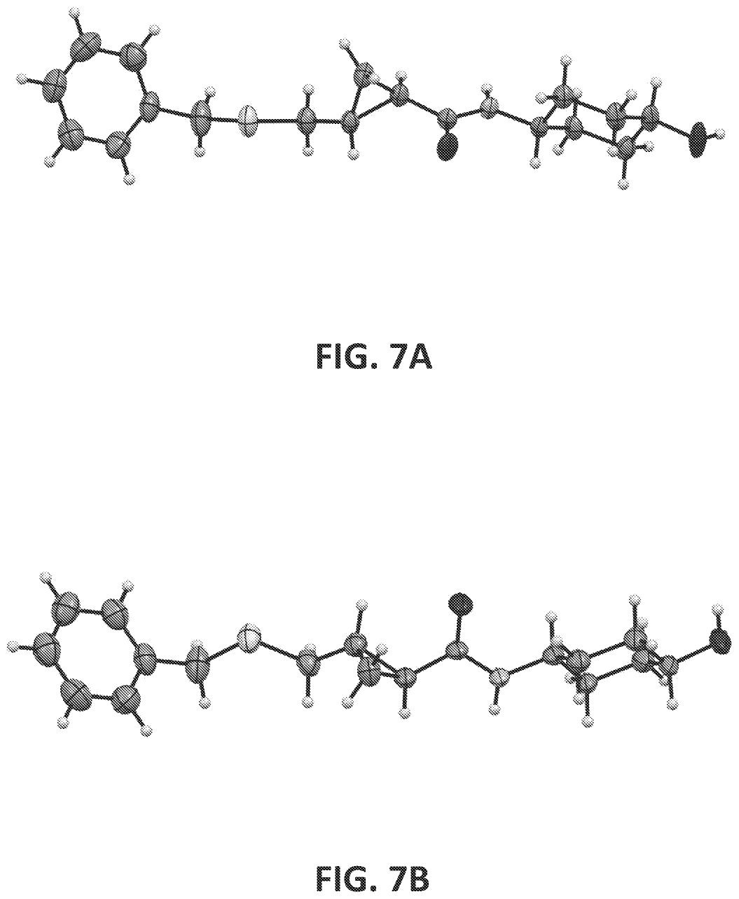

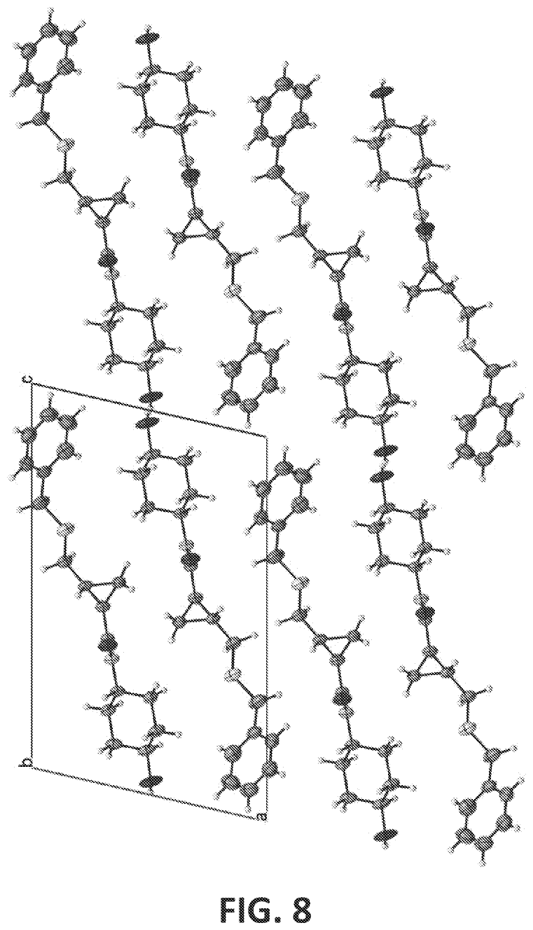

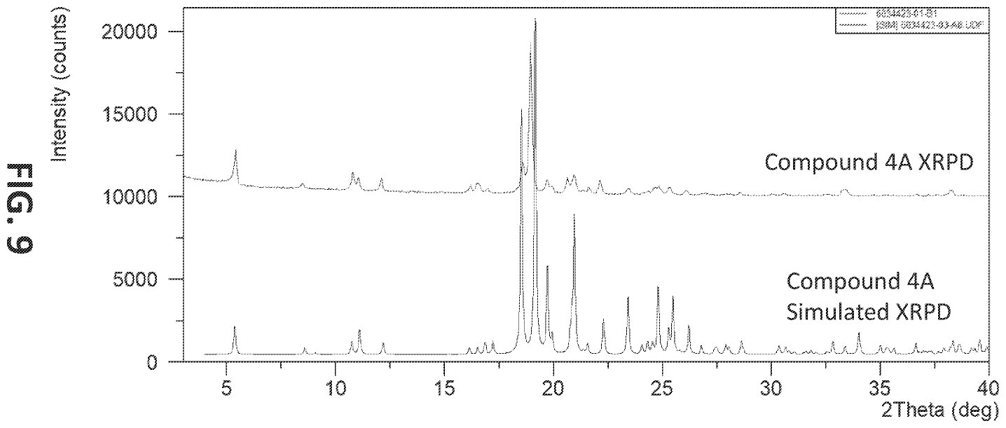

FIGS. 7A and 7B show ORTEP diagrams representative of the single-crystal x-ray crystallographic structures of Compounds 4A and 4B, respectively. Thermal ellipsoids are shown at 50% confidence interval. Hydrogen atoms are geometrically idealized. FIG. 8 shows a packing diagram for Compound 4A.

The x-ray crystallographic structure of Compound 4A displays no crystallographic disorder of any variety. The asymmetric unit cell contains only a single molecule. There are no solvent molecules present, which is likely why these crystals form readily out of multiple solvent systems with identical morphology. The molecules form a pseudo-polymeric structure connected by the amide moieties near the center of the molecule. The carbonyl oxygen (O10) forms a strong hydrogen bonding interaction with the hydrogen connected to the adjacent molecules amide nitrogen (N8). The hydrogen bonding distance, as measured by the donor-acceptor distance, is 2.887 Å. In addition, this contact only slightly deviates from the idealized hydrogen geometry as measured by linearity including the idealized H8A across the O10-N8 angle of 171.31°. The second of these hydrogen bonding interactions is a dimerization of these pseudo-polymeric structures across the terminal alcohol (O1). The donor-acceptor distance of this contact is measured to be 2.745 Å, for an even stronger interaction. This may be the result of every involved alcohol being both donor and acceptor, further polarizing each oxygen involved, especially with the zig-zag formation with an O-O-O angle of 130.72° being conducive to a trigonal planar type interaction.

The carbon bonds on either side of the sulfur atom are highly symmetrical (1.805, 1.817 Å), while the C14-S15-C16 bond angle is a sharp 101.12°, which is not uncommon for organosulfur interactions. The bonds within the cyclopropyl moiety are slightly uneven, as the longest interaction is the backbone C11-C13 bond (1.515 Å), while the adjoining bonds are asymmetrical with a longer bond on the carbon alpha to the electropositive amide carbon (C11-C12, 1.513 Å) compared to the carbon beta to the electron donating sulfur (C13-C12, 1.484 Å). The molecule as a whole, if measured across the two hydrogen atoms idealized upon the two farthest atoms, is 18.415 Å in length.

The unit cell of Compound 4A has no solvate molecules that can be crystallographically resolved and contains a total solvent-accessible void space of 0% (0.0 Å³) as calculated with a 1.2 Å probe. The total number of electrons estimated within the unit cell (F000′) is 345.54 while the total accounted for within the structure (F000) is 344.0, leaving approximately 1.54 electrons worth of density within the Fourier peaks unattributed to existing atoms, extremely inadequate to attribute to unidentified solvent molecules.

The crystal form of Compound 4A was not changed by recrystallization. FIG. 9 shows x-ray powder diffraction data for as-obtained, microcrystalline Compound 4A in comparison to simulated x-ray powder diffraction data obtained from the single crystal x-ray crystallographic data of Compound 4A. Based on the similarity of these plots, the crystal form does not change.

### EQUIVALENTS

The details of one or more embodiments of the disclosure are set forth in the accompanying description above. Although any methods and materials similar or equivalent to those described herein can be used in the practice or testing of the present disclosure, the preferred methods and materials are now described. Other features, objects, and advantages of the disclosure will be apparent from the description and from the claims. In the specification and the appended claims, the singular forms include plural referents unless the context clearly dictates otherwise. Unless defined otherwise, all technical and scientific terms used herein have the same meaning as commonly understood by one of ordinary skill in the art to which this disclosure belongs. All patents and publications cited in this specification are incorporated by reference.

The foregoing description has been presented only for the purposes of illustration and is not intended to limit the disclosure to the precise form disclosed, but by the claims appended hereto.

1. A compound of Formula (I): 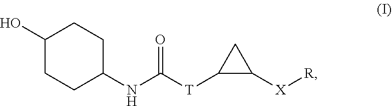
or a pharmaceutically acceptable salt thereof, wherein
T is absent, C₁-C₅ alkylene, or 2- to 5-membered heteroalkylene, wherein the C₁-C₅ alkylene or 2- to 5-membered heteroalkylene is optionally substituted with one or more R^{T};
each R^{T} independently is halogen, cyano, —OR^{T1}, —N(R^{T1})₂, oxo, C₁-C₁₀ alkyl, or C₃-C₁₀ cycloalkyl; or two R^{T}, together with the atom they attach to, form C₃-C₁₀ cycloalkyl or 3- to 10-membered heterocycloalkyl;
each R^{T1} independently is H or C₁-C₆ alkyl;
X is C₂-C₅ alkylene or 2- to 5-membered heteroalkylene, wherein the C₂-C₅ alkylene or 2- to 5-membered heteroalkylene is optionally substituted with one or more R^{X};
each R^{X} independently is halogen, cyano, OR^{X1}, —N(R^{X1})₂, oxo, C₁-C₁₀ alkyl, or C₃-C₁₀ cycloalkyl, or two R^{X}, together with the atom they attach to, form C₃-C₁₀ cycloalkyl or 3- to 10-membered heterocycloalkyl;
each R^{X1} independently is H or C₁-C₆ alkyl;
R is C₆-C₁₀ aryl or 5- to 10-membered heteroaryl, wherein the C₆-C₁₀ aryl or 5- to 10-membered heteroaryl is optionally substituted with one or more halogen, cyano, —OR^{S}, —N(R^{S})₂, C₁-C₁₀ alkyl, or C₃-C₁₀ cycloalkyl; and
each R^{S} independently is H or C₁-C₆ alkyl.

2. The compound of claim 1, being of Formula (II), (II-1), or (II-2): 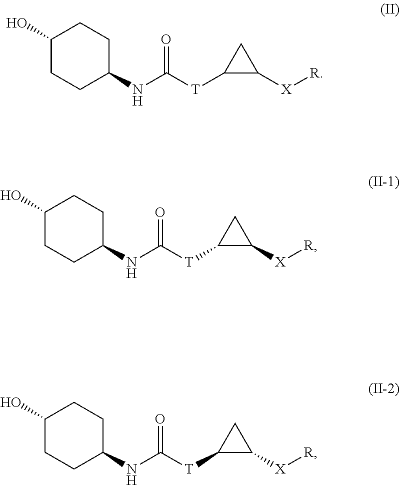
or a pharmaceutically acceptable salt thereof.

3. The compound of claim 1, being of Formula (III): 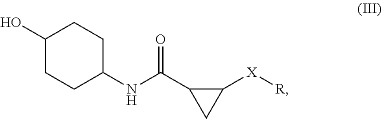
or a pharmaceutically acceptable salt thereof.

4. The compound of claim 3, being of Formula (IV), (IV-1), or (IV-2): 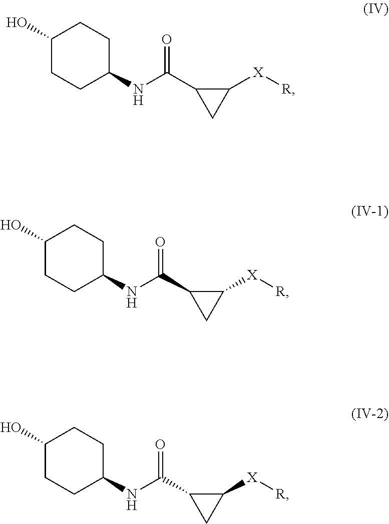
or a pharmaceutically acceptable salt thereof.

5. The compound of claim 1, wherein T is absent.

6. The compound of claim 1, wherein T is C₁-C₅ alkylene optionally substituted with one or more R^{T}.

7. (canceled)

8. The compound of claim 1, wherein X is C₂-C₅ alkylene optionally substituted with one or more R^{X}.

9. (canceled)

10. The compound of claim 1, wherein X is 2- to 5-membered heteroalkylene optionally substituted with one or more halogen, cyano, —OR^{X}, —N(R^{X})₂, or C₃-C₁₀ cycloalkyl.

11. The compound of claim 1, wherein X is —CH₂YCH₂—* or —CH₂CH₂Y —* , wherein:
* denotes attachment to R; and
Y is —O—, —S—, —S(═O)—, —S(═O)₂—, —C(R^{X})₂—, or —NR^{X}—.

12. The compound of claim 11, wherein X is —CH₂YCH₂—*, and Y is —O—, —S—, or —CH₂—.

13. The compound of claim 1, wherein X is —(CH₂)₃—.

14. The compound of claim 1, wherein R is C₆-C₁₀ aryl optionally substituted with one or more halogen, cyano, —OR^{S}, —N(R^{S})₂, or C₃-C₁₀ cycloalkyl.

15. (canceled)

16. The compound of claim 1, wherein R is phenyl.

17. The compound of claim 1, being selected from the group consisting of 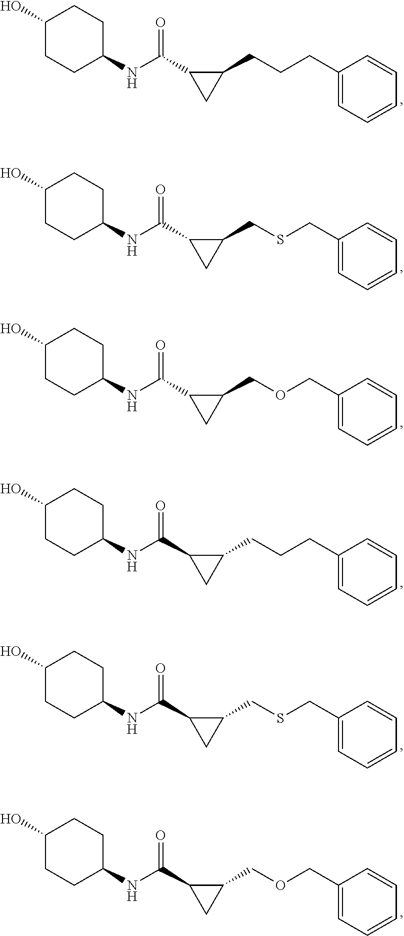
or a pharmaceutically acceptable salt thereof.

18. The compound of claim 1, being 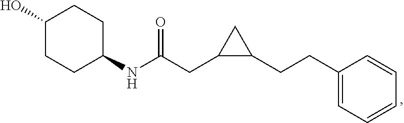 [SMILES: O=C(CC1CC1CCc1ccccc1)N[C@H]1CC[C@H](O)CC1]
or a pharmaceutically acceptable salt thereof.

19. A pharmaceutical composition, comprising the compound of claim 1 and a pharmaceutically acceptable excipient.

20. A method of treating or preventing a disease, disorder, or condition in a subject in need thereof, comprising administering the compound of claim 1 or a pharmaceutical composition comprising the compound of claim 1 and a pharmaceutically acceptable excipient to the subject.

21. (canceled)

22. (canceled)

23. The method of claim 20, wherein a therapeutically effective amount of the compound or the pharmaceutical composition is administered to the subject.

24. The method of claim 20, wherein the disease, disorder, or condition is associated with mitochondria.

25. The method of claim 20, wherein the disease, disorder, or condition is peripheral nervous system (PNS) or central nervous system (CNS) genetic or non-genetic disorder, physical damage, or chemical injury.

26. The method of claim 25, wherein the PNS or CNS genetic or non-genetic disorder is one or more conditions selected from the group consisting of a chronic neurodegenerative condition in which mitochondrial fusion, fitness, and/or trafficking is/are impaired; a disease or disorder associated with mitofusin 1 (MFN1) or mitofusin 2 (MFN2) dysfunction; a disease associated with mitochondrial fragmentation, dysfunction, and/or dysmotility; a degenerative neuromuscular condition; Charcot-Marie-Tooth disease; Amyotrophic Lateral Sclerosis; Huntington's disease; Alzheimer's disease; Parkinson's disease; hereditary motor and sensory neuropathy; autism; autosomal dominant optic atrophy (ADOA); muscular dystrophy; Lou Gehrig's disease; cancer; mitochondrial myopathy; diabetes mellitus and deafness (DAD); Leber's hereditary optic neuropathy (LHON); Leigh syndrome; subacute sclerosing encephalopathy; neuropathy; ataxia; retinitis pigmentosa, and ptosis (NARP); myoneurogenic gastrointestinal encephalopathy (MNGIE); myoclonic epilepsy with ragged red fibers (MERRF); mitochondrial myopathy, encephalomyopathy, lactic acidosis, and stroke-like symptoms (MELAS); mtDNA depletion; mitochondrial neurogastrointestinal encephalomyopathy (MNGIE); dysautonomic mitochondrial myopathy; mitochondrial channelopathy; pyruvate dehydrogenase complex deficiency (PDCD/PDH); diabetic neuropathy; chemotherapy-induced peripheral neuropathy; crush injury; spinal cord injury (SCI); traumatic brain injury; stroke; optic nerve injury; conditions that involve axonal disconnection; and any combination thereof.

27. A method of activating mitofusin in a subject, comprising administering the compound of claim 1 or a pharmaceutical composition comprising the compound of claim 1 and a pharmaceutically acceptable excipient to the subject.

28. (canceled)

29. (canceled)

30. (Cancelled)


SEQUENCE LISTING (14 sequences)

SEQ ID NO: 1 - DNA, 21 bp, Artificial Sequence
  SOD1 L38V fw
cttcactgtgaggggtaaagg

SEQ ID NO: 2 - DNA, 20 bp, Artificial Sequence
  SOD1 L38V rv
ctagggtgaacaagtatggg

SEQ ID NO: 3 - DNA, 20 bp, Artificial Sequence
  SOD1 I113T fw
tgtttagtggcatcagccct

SEQ ID NO: 4 - DNA, 22 bp, Artificial Sequence
  SOD1 I113T rv
accgcgactaacaatcaaagtg

SEQ ID NO: 5 - DNA, 24 bp, Artificial Sequence
  SOD1 L145F fw
ggtagtgattacttgacagcccaa

SEQ ID NO: 6 - DNA, 22 bp, Artificial Sequence
  SOD1 L145F rv
gttaaggggcctcagactacat

SEQ ID NO: 7 - DNA, 20 bp, Artificial Sequence
  TDP43 A382T fw
aacatgcagagggagccaaa

SEQ ID NO: 8 - DNA, 20 bp, Artificial Sequence
  TDP43 A382T rv
accctgcattggatgctgat

SEQ ID NO: 9 - DNA, 21 bp, Artificial Sequence
  FUS R521G fw
tactcgctgggttaggtagga

SEQ ID NO: 10 - DNA, 21 bp, Artificial Sequence
  FUS R521G rv 5
acgagggtaacactgggtaca

SEQ ID NO: 11 - DNA, 20 bp, Artificial Sequence
  PGRN M1L and A9D fw
ggggctagggtactgagtga

SEQ ID NO: 12 - DNA, 20 bp, Artificial Sequence
  PGRN M1L and A9D rv
tggccaatccaagatgaccc

SEQ ID NO: 13 - DNA, 21 bp, Artificial Sequence
  MAPT R406W fw
ctttctctggcacttcatctc

SEQ ID NO: 14 - DNA, 21 bp, Artificial Sequence
  MAPT R406W rv
cctctccacaattattgaccg